SEQUENCE LISTING (183 sequences)

SEQ ID NO: 1 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1251 aa, synthetic construct
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLLKRPAATKKAGQAKKKKGSYPYDVPDYAYPYDVPDYAYPYDVPDYA

SEQ ID NO: 4 - protein, 1273 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 5 - protein, 1251 aa, synthetic construct
MLFQDFTHLYPLSKTVRFELKPIGRTLEHIHAKNFLSQDETMADMYQKVKVILDDYHRDFIADMMGEVKLTKLAEFYDVYLKFRKNPKDDELQKQLKDLQAVLRKESVKPIGNGGKYKAGHDRLFGAKLFKDGKELGDLAKFVIAQEGKSSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIKHHTARHDDESVQAGKLGQYFKHGLAGVDNPIQKIHNNHSTIKGFLERERPAGERALPKIKSGKNPEMTQLRQLKELLDNALNVAHFAKLLMTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMIYKLLPGPNKMLPRVFFAKSNLDYYNPSAELLDKYAQGTHKKGDNFNLKDCHALIDFFKAGINKHPEWQNFGFKFSPTSSYRDLSDFYREVEPQGYQVKFVDINADYIDELVEQGQLYLFQIYNKDFSPKAHGKPNLHTLYFRALFSEDNLANPIYKLNGEAQIFYRKASLGMNETTIHRAGEILENKNPDNPKERVFTYDIIKDRRYTQDKFMLHVPITMNFGVQGMTIKEFNKKVNQSIRQYDDVNVIGIDRGERHLLYLTVINSKGEILEQRSLNDITTASANGTQMTTPYHKILDKREIERLNARVGWGEIETIKELKSGYLSHVVHQVSQLMLKYNAIVVLEDLNFGFKRGRFKVEKQIYQNFENALIKKLNHLELKDKADDEIGSYKNALQLTNNFTDLKNIGKQTGFLFYVPAWNTSKIDPETGFVDLLKPRYENIAQSQAFFGKFDKICYNADKDYFEFHIDYAKFTDKAKNSRQTWTICSHGDKRYVYDKTANQNKGATKGINVNDELKSLFARYHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 6 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 7 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHKFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 8 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDIKTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQRFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 9 - protein, 1306 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDIKTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNKSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQRFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDIKTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHKFIPLFKQILSDRNKLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQRFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 11 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFGGKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 12 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTYKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 13 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 14 - protein, 1280 aa, synthetic construct
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 15 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 16 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGRPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYLLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 17 - RNA, 10 nt, synthetic construct
  source 1..10: mol_type = other RNA
  modified_base 1..10: mod_base = OTHER; note = 2-prime-hydroxy RNA nucleotides
aagacctttt

SEQ ID NO: 18 - RNA, 25 nt, synthetic construct
  source 1..25: mol_type = other RNA
  modified_base 1..25: mod_base = OTHER; note = 2-prime-hydroxy RNA nucleotides
atgtgtttttgtcaaaagacctttt

SEQ ID NO: 19 - RNA, 60 nt, synthetic construct
  source 1..60: mol_type = other RNA
  modified_base 1..60: mod_base = OTHER; note = 2-prime-hydroxy RNA nucleotides
aggccagcttgccggttttttagtcgtgctgcttcatgtgtttttgtcaaaagacctttt

SEQ ID NO: 20 - DNA, 10 bp, synthetic construct
  source 1..10: mol_type = other DNA
aagacctttt

SEQ ID NO: 21 - DNA, 25 bp, synthetic construct
  source 1..25: mol_type = other DNA
atgtgtttttgtcaaaagacctttt

SEQ ID NO: 22 - DNA, 60 bp, synthetic construct
  source 1..60: mol_type = other DNA
aggccagcttgccggttttttagtcgtgctgcttcatgtgtttttgtcaaaagacctttt

SEQ ID NO: 23 - DNA, 20 bp, synthetic construct
  source 1..20: mol_type = other DNA
tttttgtcaaaagacctttt

SEQ ID NO: 24 - DNA, 30 bp, synthetic construct
  source 1..30: mol_type = other DNA
gcttcatgtgtttttgtcaaaagacctttt

SEQ ID NO: 25 - DNA, 50 bp, synthetic construct
  source 1..50: mol_type = other DNA
gccggttttttagtcgtgctgcttcatgtgtttttgtcaaaagacctttt

SEQ ID NO: 26 - DNA, 40 bp, synthetic construct
  source 1..40: mol_type = other DNA
tagtcgtgctgcttcatgtgtttttgtcaaaagacctttt

SEQ ID NO: 27 - DNA, 20 bp, synthetic construct
  source 1..20: mol_type = other DNA
  modified_base 1^2: mod_base = OTHER; note = phosphorothioate
  modified_base 2^3: mod_base = OTHER; note = phosphorothioate
ccgaagttttcttcggtttt

SEQ ID NO: 28 - DNA, 25 bp, synthetic construct
  source 1..25: mol_type = other DNA
  modified_base 1^2: mod_base = OTHER; note = phosphorothioate
  modified_base 2^3: mod_base = OTHER; note = phosphorothioate
tttttccgaagttttcttcggtttt

SEQ ID NO: 29 - DNA, 30 bp, synthetic construct
  source 1..30: mol_type = other DNA
  modified_base 1^2: mod_base = OTHER; note = phosphorothioate
  modified_base 2^3: mod_base = OTHER; note = phosphorothioate
aacgctttttccgaagttttcttcggtttt

SEQ ID NO: 30 - DNA, 41 bp, synthetic construct
  source 1..41: mol_type = other DNA
  modified_base 1^2: mod_base = OTHER; note = phosphorothioate
  modified_base 2^3: mod_base = OTHER; note = phosphorothioate
gcgttgttttcaacgctttttccgaagttttcttcggtttt

SEQ ID NO: 31 - DNA, 62 bp, synthetic construct
  source 1..62: mol_type = other DNA
  modified_base 1^2: mod_base = OTHER; note = phosphorothioate
  modified_base 2^3: mod_base = OTHER; note = phosphorothioate
ggcttcttttgaagcctttttgcgttgttttcaacgctttttccgaagttttcttcggtttt

SEQ ID NO: 32 - DNA, 25 bp, synthetic construct
  source 1..25: mol_type = other DNA
  modified_base 1^2: mod_base = OTHER; note = phosphorothioate
  modified_base 2^3: mod_base = OTHER; note = phosphorothioate
atgtgtttttgtcaaaagacctttt

SEQ ID NO: 33 - DNA, 25 bp, synthetic construct
  source 1..25: mol_type = other DNA
aaaaaaaaaaaaaaaaaaaaaaaaa

SEQ ID NO: 34 - DNA, 25 bp, synthetic construct
  source 1..25: mol_type = other DNA
ttttttttttttttttttttttttt

SEQ ID NO: 35 - RNA, 25 nt, synthetic construct
  source 1..25: mol_type = other RNA
  modified_base 1..2: mod_base = OTHER; note = 2-prime-O-methyl RNA nucleotides
  modified_base 3..25: mod_base = OTHER; note = 2-prime-hydroxy RNA nucleotides
  modified_base 1^2: mod_base = OTHER; note = phosphorothioate
  modified_base 2^3: mod_base = OTHER; note = phosphorothioate
atgtgtttttgtcaaaagacctttt

SEQ ID NO: 36 - RNA, 25 nt, synthetic construct
  source 1..25: mol_type = other RNA
  modified_base 1..2: mod_base = OTHER; note = 2-prime-O-methyl RNA nucleotides
  modified_base 3..25: mod_base = OTHER; note = 2-prime-hydroxy RNA nucleotides
  modified_base 1^2: mod_base = OTHER; note = phosphorothioate
  modified_base 2^3: mod_base = OTHER; note = phosphorothioate
aaaaaaaaaaaaaaaaaaaaaaaaa

SEQ ID NO: 37 - RNA, 25 nt, synthetic construct
  source 1..25: mol_type = other RNA
  modified_base 1..2: mod_base = OTHER; note = 2-prime-O-methyl RNA nucleotides
  modified_base 3..25: mod_base = OTHER; note = 2-prime-hydroxy RNA nucleotides
  modified_base 1^2: mod_base = OTHER; note = phosphorothioate
  modified_base 2^3: mod_base = OTHER; note = phosphorothioate
ttttttttttttttttttttttttt

SEQ ID NO: 38 - RNA, 19 nt, synthetic construct
  source 1..19: mol_type = other RNA
taatttctactcttgtaga

SEQ ID NO: 39 - RNA, 21 nt, synthetic construct
  source 1..21: mol_type = other RNA
taattttctacttgttgtaga

SEQ ID NO: 40 - RNA, 20 nt, synthetic construct
  source 1..20: mol_type = other RNA
taatttctactaagtgtaga

SEQ ID NO: 41 - RNA, 15 nt, synthetic construct
  source 1..15: mol_type = other RNA
atctacaacagtaga

SEQ ID NO: 42 - RNA, 15 nt, synthetic construct
  source 1..15: mol_type = other RNA
atctacaaaagtaga

SEQ ID NO: 43 - RNA, 20 nt, synthetic construct
  source 1..20: mol_type = other RNA
ggaatttctactcttgtaga

SEQ ID NO: 44 - RNA, 10 nt, synthetic construct
  source 1..10: mol_type = other RNA
taatttctac

SEQ ID NO: 45 - RNA, 12 nt, synthetic construct
  source 1..12: mol_type = other RNA
taattttctact

SEQ ID NO: 46 - RNA, 10 nt, synthetic construct
  source 1..10: mol_type = other RNA
taatttctac

SEQ ID NO: 47 - skipped

SEQ ID NO: 48 - RNA, 11 nt, synthetic construct
  source 1..11: mol_type = other RNA
ggaatttctac

SEQ ID NO: 49 - RNA, 10 nt, synthetic construct
  source 1..10: mol_type = other RNA
taattcccac

SEQ ID NO: 50-51 - skipped (2 entries)

SEQ ID NO: 52 - RNA, 35 nt, synthetic construct
  source 1..35: mol_type = other RNA
gttaagttatatagaataatttctactgttgtaga

SEQ ID NO: 53 - RNA, 36 nt, synthetic construct
  source 1..36: mol_type = other RNA
ctctacaactgataaagaatttctacttttgtagat

SEQ ID NO: 54 - RNA, 36 nt, synthetic construct
  source 1..36: mol_type = other RNA
gtctggccccaaattttaatttctactgttgtagat

SEQ ID NO: 55 - RNA, 20 nt, synthetic construct
  source 1..20: mol_type = other RNA
taatttctactgttgtagat

SEQ ID NO: 56 - protein, 236 aa, synthetic construct
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 57 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 58 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 59 - protein, 208 aa, synthetic construct
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 60 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 61 - protein, 229 aa, synthetic construct [sequence omitted]

SEQ ID NO: 62 - protein, 198 aa, synthetic construct [sequence omitted]

SEQ ID NO: 63 - protein, 197 aa, synthetic construct
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTCT

SEQ ID NO: 64 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 65 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 66 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 67 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 68 - protein, 365 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 69 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 70 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 71 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 72 - protein, 166 aa, synthetic construct; identical to SEQ ID NO: 67

SEQ ID NO: 73 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 74 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 75 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 76 - protein, 166 aa, synthetic construct; identical to SEQ ID NO: 71

SEQ ID NO: 77 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 78 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 79 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 80 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 81 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 82 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 83 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 84 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 85 - protein, 28 aa, synthetic construct
TKKPYSVEKFKLNFQMPTLASGWDVNKE

SEQ ID NO: 86 - protein, 28 aa, synthetic construct
TKKPYSVEKFKLNFQRPTLASGWDVNKE

SEQ ID NO: 87 - protein, 28 aa, synthetic construct
TQKPYSDEKFKLNFENSTLANGWDKNKE

SEQ ID NO: 88 - protein, 28 aa, synthetic construct
TKKPFSTEKVKLNFNRSTLLNGWDRNKE

SEQ ID NO: 89 - protein, 28 aa, synthetic construct
TQKPYSKDKFKLYFQNPQFMGGWDKDKE

SEQ ID NO: 90 - protein, 28 aa, synthetic construct
SQKPFSTEKYKLNFGNPTLLNGWDLNKE

SEQ ID NO: 91 - protein, 28 aa, synthetic construct
HEIIKDRRFTSDKFFFHVPITLNYQAAN

SEQ ID NO: 92 - protein, 28 aa, synthetic construct
HEIIKDRRFTSDKFLFHVPITLNYQAAN

SEQ ID NO: 93 - protein, 27 aa, synthetic construct
YDLIKDKRFTEDKFFFHCPITINFKSS

SEQ ID NO: 94 - protein, 27 aa, synthetic construct
YDIVKDKRYSKDKFTLHIPITMNFGVD

SEQ ID NO: 95 - protein, 27 aa, synthetic construct
YDVYKDKRFSEDQYELHIPIAINKCPK

SEQ ID NO: 96 - protein, 28 aa, synthetic construct
YDIIKDKRYTQDKFMLHVPITMNFGVQG

SEQ ID NO: 97 - protein, 49 aa, synthetic construct
FKLNFQMPTLASGWDVNKEKNNGAILFVKNGLYYLGIMPKQKGRYKALS

SEQ ID NO: 98 - protein, 48 aa, synthetic construct
IKINFDAPTLLNGWDLNKESDNKSIILRKDGNFYLAIMHPKHTKVFDC

SEQ ID NO: 99 - protein, 50 aa, synthetic construct
FFHVPITLNYQAANSPSKFNQRVNAYLKEHPETPIIGIDRGERNLIYITV

SEQ ID NO: 100 - protein, 49 aa, synthetic construct
FFHVPISLNFKSQGVVRFNDKINGLLAAQDDVHVIGIDRGERHLLYYTV

SEQ ID NO: 101 - protein, 21 aa, synthetic construct
KFTTYFSGFYENRKNVFSAED

SEQ ID NO: 102 - protein, 21 aa, synthetic construct
GWTTYFKGFHENRKNVYSSND

SEQ ID NO: 103 - protein, 21 aa, synthetic construct
NFYTYFTSYNKVRENLYSDKE

SEQ ID NO: 104 - protein, 21 aa, synthetic construct
GFTTAFTGFFDNRENMFSEEA

SEQ ID NO: 105 - protein, 21 aa, synthetic construct
KFSTYFTGFHDNRKNMYSDED

SEQ ID NO: 106 - protein, 21 aa, synthetic construct
KFTTYFSGFHENRKNMYTAEA

SEQ ID NO: 107 - protein, 21 aa, synthetic construct
VDYKLLPVPNQMLPKVFFAKS

SEQ ID NO: 108 - protein, 21 aa, synthetic construct
MYYDYFPDAAKMIPKCSTQLK

SEQ ID NO: 109 - protein, 21 aa, synthetic construct
IVYKLLPGANKMLPKVFFSAK

SEQ ID NO: 110 - protein, 21 aa, synthetic construct
INYKLLPGPNKMLPKVFFSKK

SEQ ID NO: 111 - protein, 21 aa, synthetic construct
MIYKLLPGPNKMLPRVFFAKS

SEQ ID NO: 112 - protein, 21 aa, synthetic construct
MNYKLLSGANKMLPKVFFSKK

SEQ ID NO: 113 - protein, 21 aa, synthetic construct
TGFANLFYMKCENVEKSKRFF

SEQ ID NO: 114 - protein, 21 aa, synthetic construct
GFVDPFVWKTIKNHESRKHFL

SEQ ID NO: 115 - protein, 21 aa, synthetic construct
TGFVNQLYPKYESVSKSQEFF

SEQ ID NO: 116 - protein, 13 aa, synthetic construct
SLLKPRYENIASS

SEQ ID NO: 117 - protein, 21 aa, synthetic construct
TGFVDLLKPRYENIAQSQAFF

SEQ ID NO: 118 - protein, 21 aa, synthetic construct
TGFVDFLKPKYESMAKSKVFF

SEQ ID NO: 119 - protein, 21 aa, synthetic construct
RKNVFSAEDISTAIPHRIVQD

SEQ ID NO: 120 - protein, 21 aa, synthetic construct
RKNVYSSNDIPTSIIYRIVDD

SEQ ID NO: 121 - protein, 21 aa, synthetic construct
RENLYSDKEKSSTVAYRLINE

SEQ ID NO: 122 - protein, 21 aa, synthetic construct
RENMFSEEAKSTSIAFRCINE

SEQ ID NO: 123 - protein, 21 aa, synthetic construct
RKNMYSDEDKHTAIAYRLIHE

SEQ ID NO: 124 - protein, 21 aa, synthetic construct
RKNMYTAEAQSTAIANRLMND

SEQ ID NO: 125 - protein, 21 aa, synthetic construct
TAHIIASLPHRFIPLFKQILS

SEQ ID NO: 126 - protein, 21 aa, synthetic construct
QINDKTLKKYKMSVLFKQILS

SEQ ID NO: 127 - protein, 20 aa, synthetic construct
KANGFRKIPKMKMLYKQILS

SEQ ID NO: 128 - protein, 14 aa, synthetic construct
KLPKFKPLYKQVLS

SEQ ID NO: 129 - protein, 13 aa, synthetic construct
IAKLRPLHKQILS

SEQ ID NO: 130 - protein, 17 aa, synthetic construct
KAKGFPRFMPLFKQILS

SEQ ID NO: 131 - protein, 21 aa, synthetic construct
LFKQILSDRNTLSFILEEFKS

SEQ ID NO: 132 - protein, 21 aa, synthetic construct
LFKQILSDTESKSFVIDKLED

SEQ ID NO: 133 - protein, 19 aa, synthetic construct
LYKQILSDREESFIDEFQS

SEQ ID NO: 134 - protein, 21 aa, synthetic construct
LYKQVLSDRESLYKQVLSDRE

SEQ ID NO: 135 - protein, 21 aa, synthetic construct
LHKQILSDGMGVSFLPSKFAD

SEQ ID NO: 136 - protein, 21 aa, synthetic construct
LFKQILSDRETHSFLPDAFEN

SEQ ID NO: 137 - protein, 21 aa, synthetic construct
TGIAEKAVYQQFEKMLIDKLN

SEQ ID NO: 138 - protein, 20 aa, synthetic construct
FKVEKQVYQKLEKMLIEKLN

SEQ ID NO: 139 - protein, 20 aa, synthetic construct
EKVEKQVYQKFEKMLIDKLN

SEQ ID NO: 140 - protein, 20 aa, synthetic construct
VKVEKQVYQKFEKMLIDKLN

SEQ ID NO: 141 - protein, 20 aa, synthetic construct
FKVEKQIYQNFENALIKKLN

SEQ ID NO: 142 - protein, 21 aa, synthetic construct
RFKVEKQVYQKFEKALIDKLN

SEQ ID NO: 143 - protein, 21 aa, synthetic construct
VLENLNFGFKSKRTGIAEKAV

SEQ ID NO: 144 - protein, 20 aa, synthetic construct
VFEDLNFGFKRGRFKVEKQV

SEQ ID NO: 145 - protein, 20 aa, synthetic construct
CLEDLNFGFKRGRQKVEKQV

SEQ ID NO: 146 - protein, 20 aa, synthetic construct
ALEDLNSGFKNSRVKVEKQV

SEQ ID NO: 147 - protein, 20 aa, synthetic construct
VLEDLNFGFKRGRFKVEKQI

SEQ ID NO: 148 - protein, 20 aa, synthetic construct
CLEDLNFGFKRGRFKVEKQV

SEQ ID NO: 149 - protein, 50 aa, synthetic construct
GTVTTTEHENALLRSFDKFTTYFSGFYENRKNVFSAEDISTAIPHRIVQD

SEQ ID NO: 150 - protein, 50 aa, synthetic construct
YSASEKEEKTQVIKLFSRFATSFKDYFKNRANCFSADDISSSSCHRIVND

SEQ ID NO: 151 - protein, 50 aa, synthetic construct
TKKPYSVEKFKLNFQMPTLASGWDVNKEKNNGAILFVKNGLYYLGIMPKQ

SEQ ID NO: 152 - protein, 50 aa, synthetic construct
TQKPYSTKKIKLNFGIPTLADGWSKSKEYSNNAIILMRDNLYYLGIFNAK

SEQ ID NO: 153 - protein, 49 aa, synthetic construct
PNVITKEVSHEIIKDRRFTSDKFFFHVPITLNYQAANSPSKFNQRVNAY

SEQ ID NO: 154 - protein, 49 aa, synthetic construct
KNVVGHHEAATNIVKDYRYTYDKYFLHMPITINFKANKTGFINDRILQY

SEQ ID NO: 155 - protein, 21 aa, synthetic construct
MIYNLLPGPNKMIPKVFLSSK

SEQ ID NO: 156 - protein, 21 aa, synthetic construct
TGFVNIFKFKDLTVDAKREFI

SEQ ID NO: 157 - protein, 21 aa, synthetic construct
RFATSFKDYFKNRANCFSADD

SEQ ID NO: 158 - protein, 21 aa, synthetic construct
RANCFSADDISSSSCHRIVND

SEQ ID NO: 159 - protein, 21 aa, synthetic construct
QKNKENKNLYKLQKLHKQILC

SEQ ID NO: 160 - protein, 21 aa, synthetic construct
LHKQILCIADTSYEVPYKFES

SEQ ID NO: 161 - protein, 20 aa, synthetic construct
FKVERQVYQKFETMLINKLN

SEQ ID NO: 162 - protein, 20 aa, synthetic construct
AMEDLSYGFKKGRFKVERQV

SEQ ID NO: 163 - protein, 21 aa, synthetic construct
KLNFQMPTLASGWDVNREKNN

SEQ ID NO: 164 - protein, 21 aa, synthetic construct
KLNFENSTLANGWDKNKEPDN

SEQ ID NO: 165 - protein, 21 aa, synthetic construct
KLNFNRSTLLNGWDRNKETDN

SEQ ID NO: 166 - protein, 21 aa, synthetic construct
KLYFQNPQFMGGWDKDKETDY

SEQ ID NO: 167 - protein, 21 aa, synthetic construct
KLNFGNPTLLNGWDLNKEKDN

SEQ ID NO: 168 - protein, 21 aa, synthetic construct
KINFDAPTLLNGWDLNKESDN

SEQ ID NO: 169 - protein, 21 aa, synthetic construct
KLNFGIPTLADGWSKSKEYSN

SEQ ID NO: 170 - protein, 21 aa, synthetic construct
PTLASGWDVNKEKNNGAILFV

SEQ ID NO: 171 - protein, 21 aa, synthetic construct
STLANGWDKNKEPDNTAILFI

SEQ ID NO: 172 - protein, 21 aa, synthetic construct
STLLNGWDRNKETDNLGVLLL

SEQ ID NO: 173 - protein, 21 aa, synthetic construct
PQFMGGWDKDKETDYRATILR

SEQ ID NO: 174 - protein, 21 aa, synthetic construct
PTLLNGWDLNKEKDNFGVILQ

SEQ ID NO: 175 - protein, 21 aa, synthetic construct
PTLLNGWDLNKESDNKSIILR

SEQ ID NO: 176 - protein, 21 aa, synthetic construct
PTLADGWSKSKEYSNNAIILM

SEQ ID NO: 177 - protein, 21 aa, synthetic construct
VALIRSVLQMRNSNAATGEDY

SEQ ID NO: 178 - protein, 21 aa, synthetic construct
TSVLNTILQMRNSKTGTELDY

SEQ ID NO: 179 - protein, 21 aa, synthetic construct
YRLLQQTLQMRNSTSDGTRDY

SEQ ID NO: 180 - protein, 21 aa, synthetic construct
MALMSLMLQMRNSTGRTDVDF

SEQ ID NO: 181 - protein, 21 aa, synthetic construct
MYLLKTLLALRYSNASSDEDF

SEQ ID NO: 182 - protein, 21 aa, synthetic construct
YWLLRLTLSLRHSVTGTDEDF

SEQ ID NO: 183 - protein, 21 aa, synthetic construct
FEIFRLTVQMRNSLSELEDDR

(19) United States
(12) Patent Application Publication
(10) Pub. No.: US 2025/0025572 A1
(43) Pub. Date: Jan. 23, 2025

(54) ENGINEERED CRISPR/CAS12A EFFECTOR PROTEINS, AND USES THEREOF

(71) Applicant: Editas Medicine, Inc., Cambridge, MA (US)

(72) Inventor: John Anthony Zuris, Boston, MA (US)

(21) Appl. No.: 18/712,894

(22) PCT Filed: Nov. 28, 2022
(86) PCT No.: PCT/US2022/080510

§ 371 Date: May 23, 2024

Related U.S. Application Data
(60) Provisional application No. 63/301,953, filed on Jan. 21, 2022.
(60) Provisional application No. 63/301,955, filed on Jan. 21, 2022.
(60) Provisional application No. 63/301,956, filed on Jan. 21, 2022.
(60) Provisional application No. 63/283,690, filed on Nov. 29, 2021.
(60) Provisional application No. 63/283,770, filed on Nov. 29, 2021.
(60) Provisional application No. 63/283,965, filed on Nov. 29, 2021.

(51) Int. Cl. A61K 48/00; C12N 9/22; C12N 9/80; C12N 15/11; C12N 15/86

(52) U.S. Cl. CPC A61K 48/005; C12N 9/22; C12N 9/80; C12N 15/11; C12N 15/86; C12N 2310/20; C12N 2750/14143; C12Y 305/04001; C12Y 305/04002

(57) ABSTRACT

The present disclosure relates to Cast 2a effector proteins with increased activity compared to previously described Cas12a effector proteins. The present disclosure also relates to fusion proteins comprising a Cas12a effector protein fused to a deaminase with increased activity compared to previously described fusion proteins comprising a Cast 12a effector protein fused to a deaminase. Systems and methods of their use are also disclosed.

### CROSS-REFERENCE TO RELATED APPLICATION

This application claims the benefit of U.S. Provisional Applications 63/283,690, filed Nov. 29, 2021, 63/283,770, filed Nov. 29, 2021, 63/283,965, filed Nov. 29, 2021, 63/301,953, filed Jan. 21, 2022, 63/301,955, filed Jan. 21, 2022, and 63/301,956, filed Jan. 21, 2022, the contents of each of which are hereby incorporated by reference in their entireties.

### SEQUENCE LISTING

The instant application contains a Sequence Listing, which has been submitted electronically in .xml format and is hereby incorporated by reference in its entirety. Said xml copy, created on Nov. 28, 2022 is named 2003080-0237.xml and is 202,812 bytes in size.

### BACKGROUND

Type V CRISPR/Cas12a effector proteins (also referred to as Cpf1 effector proteins) have been described as an alternative to Cas9 effector proteins for genome editing applications (Zetsche et al., Cell 163:759-771 (2015): Shmakov et al., Mol Cell. 60 (3): 385-97 (2015): Kleinstiver et al., Nat Biotechnol 34 (8): 869-74 (2016): Kim et al., Nat Biotechnol 34 (8): 863-8 (2016)). Cas12a effector proteins possess a number of potentially advantageous properties that include, but are not limited to: recognition of T-rich protospacer-adjacent motif (PAM) sequences, relatively greater genome-wide specificities in human cells compared to wild-type Streptococcus pyogenes Cas9 (SpCas9), an endoribonuclease activity to process pre-crRNAs that simplifies the simultaneous targeting of multiple sites (multiplexing), DNA endonuclease activity that generates a 5′ DNA overhang (rather than a blunt double-strand break as observed with SpCas9), and cleavage of the protospacer DNA sequence on the end most distal from the PAM (compared with cleavage at the PAM proximal end of the protospacer as is observed with SpCas9).

Given these capabilities there is a need to develop Cas12a effector proteins that provide a suitable alternative to Cas9 effector proteins for genome editing applications in humans.

### SUMMARY

The present disclosure provides strategies, systems, compositions, and methods related to engineered Type V CRISPR/Cas12a effector proteins and variants thereof with increased activity(ies) for altering a cell, e.g., altering a structure, e.g., altering a sequence, of a target nucleic acid of a cell, compared to other Type V CRISPR/Cas12a effector proteins described in the art. For example, in some embodiments, the present disclosure provides for strategies, systems, compositions, and methods related to engineered Cas12a effector proteins and variants thereof with increased activity(ies) for introducing double strand and/or single strand breaks in a target nucleic sequence, compared to other Cas12a effector proteins described in the art.

Among other things, the present disclosure provides strategies, systems, compositions, and methods related to engineered Cas12a effector proteins and variants thereof that are fused to one or more heterologous protein domains (or “fusion proteins”). For example, in some embodiments, Cas12a effector proteins may be fused to one or more heterologous protein domains such as a deaminase or catalytic domain for base editing. The fusion proteins provided herein exhibit increased activity(ies) compared to fusion proteins known in the art.

The disclosed Cas12a effector proteins, and related strategies, systems, compositions, and methods, present several advantages compared to other Cas12a effector proteins known in the art. For example, in some embodiments, the described Cas12a effector proteins, and related strategies, systems, compositions, and method, create a single and/or double strand break in a target and/or non-target nucleic sequence with higher efficiency compared to other Cas12a effector proteins known in the art. Moreover, in some embodiments, the described Cas12a effector proteins, and related strategies, systems, compositions, and method, alter the genomes of at least a plurality of cells at a higher rate compared to other Cas12a effector proteins known in the art.

### BRIEF DESCRIPTION OF THE DRAWING

The teachings described herein will be more fully understood from the following description of various exemplary embodiments, when read together with the accompanying drawing. It should be understood that the drawing described below is for illustration purposes only and is not intended to limit the scope of the present teachings in any way.

FIG. 1A shows a sequence alignment of a conserved region between a wild-type FnCas12a sequence, a wild-type Lb2Cas12a sequence, a wild-type LbCas12a sequence and a wild-type MbCas12a sequence and an exemplary AsCas12a sequence to illustrate amino acid positions in FnCas12a, Lb2Cas12a, LbCas12a and MbCas12a corresponding to the AsCas12a positions M537 and F870 for substitutions M537R and F870L.

FIG. 1B shows a sequence alignment of a conserved region between a wild-type AsCas12a sequence and an exemplary MG29-1 sequence to illustrate positions in MG29-1 corresponding to the AsCas12a positions M537 and F870 for substitutions M537R and F870L.

FIG. 2 shows a wild-type FnCas12a sequence, a wild-type Lb2Cas12a sequence, a wild-type LbCas12a sequence, a wild-type MbCas12a sequence, an exemplary MG29-1 sequence, and an exemplary AsCas12a sequence to illustrate amino acid substitutions in FnCas12a, Lb2Cas12a, LbCas12a, MbCas12a, and MG29-1 corresponding to the AsCas12a substitution E174R.

FIG. 3 shows sequence alignments between a wild-type AsCas12a sequence, a wild-type FnCas12a sequence, a wild-type LbCas12a sequence, a wild-type MbCas12a sequence, an exemplary MG29-1 sequence, and a wild-type Lb2Cas12a sequence to illustrate substitutions in AsCas12a, FnCas12a, LbCas12a, MbCas12a, and MG29-1 corresponding to the Lb2Cas12a substitutions Q571K and C1003Y.

FIG. 4A and FIG. 4B show sequence alignments between a wild-type FnCas12a sequence, a wild-type Lb2Cas12a sequence, a wild-type LbCas12a sequence, a wild-type MbCas12a sequence, an exemplary MG29-1 sequence, and a wild-type AsCas12a sequence to illustrate substitutions in FnCas12a, Lb2Cas12a, LbCas12a, MbCas12a, and MG29-1 corresponding to the AsCas12a substitutions S186K, R301K, T315R, and Q1014R.

FIG. 5 shows sequence alignments between a wild-type FnCas12a sequence, a wild-type Lb2Cas12a sequence, a wild-type LbCas12a sequence, a wild-type MbCas12a sequence, an exemplary MG29-1 sequence, and a wild-type AsCas12a sequence to illustrate substitutions in FnCas12a, Lb2Cas12a, LbCas12a, MbCas12a, and MG29-1 corresponding to the AsCas12a substitutions K1000G and S1001G.

FIG. 6 shows percent (%) knock out (KO) of a target gene (TRAC) as measured by flow cytometry after administering a variety of ribonucleic protein (RNP) complexes, each comprising an exemplary AsCas12a variant, to target cells at varying RNP concentrations.

FIG. 7 depicts an illustration of an exemplary AsCas12a variant comprising amino acid substitutions at multiple positions, in accordance with embodiments of the present disclosure.

FIG. 8 shows a sequence alignment of a highly conversed region between wild-type ErCas12a (MAD7) and an exemplary AsCas12a sequence to illustrate amino acid substitutions in MAD7 corresponding to the AsCas12a substitutions E174R, M537R, and F870L.

FIG. 9 shows sequence alignments between a wild-type ErCas12a (MAD7) sequence and a wild-type Lb2Cas12a sequence to illustrate substitutions in MAD7 corresponding to the Lb2Cas12a substitutions Q571K and C1003Y.

FIG. 10 shows sequence alignments between a wild-type ErCas12a (MAD7) sequence and a wild-type AsCas12a sequence to illustrate substitutions in MAD7 corresponding to the AsCas12a substitutions E174R, S186K, R301K, T315R, and Q1014R.

FIG. 11 shows sequence alignments between a wild-type ErCas12a (MAD7) sequence and a wild-type AsCas12a sequence to illustrate substitutions in MAD7 corresponding to the AsCas12a substitutions K1000G and S1001G.

FIG. 12A and FIG. 12B show sequence alignments between a wild-type FnCas12a sequence, a wild-type Lb2Cas12a sequence, a wild-type LbCas12a sequence, a wild-type MbCas12a sequence, an exemplary MG29-1 sequence, a wild-type ErCas12a and a wild-type AsCas12a sequence to illustrate substitutions in FnCas12a, Lb2Cas12a, LbCas12a, MbCas12a, MG29-1, and ErCas12a (MAD7) corresponding to the AsCas12a substitutions E174R, S542R, K548R, and R1226A.

### DETAILED DESCRIPTION

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “cancer” (also used interchangeably with the term “neoplastic”), as used herein, refers to cells having the capacity for autonomous growth, e.g., an abnormal state or condition characterized by rapidly proliferating cell growth. Cancerous disease states may be categorized as pathologic, e.g., characterizing or constituting a disease state, e.g., malignant tumor growth, or may be categorized as non-pathologic, e.g., a deviation from normal but not associated with a disease state, e.g., cell proliferation associated with wound repair.

The terms “CRISPR/Cas effector protein”, “Cas enzyme”, “CRISPR enzyme”, “CRISPR protein”, “Cas protein” and “CRISPR/Cas” are generally used interchangeably and at all points of reference herein refer by analogy to new CRISPR/Cas effector proteins further described in this application, unless otherwise apparent, such as by specific reference to Cas12a or Cpf1. In some embodiments, a CRISPR/Cas effector protein is part of a fusion protein comprising one or more heterologous protein domains (e.g., about or more than about 1, 2, 3, 4, 5, 6, 7, 8, 9, 10, or more domains in addition to the CRISPR enzyme). In some embodiments, one or more heterologous protein domains comprises a deaminase. In some embodiments, one or more heterologous protein domains comprises a reverse transcriptase domain. In some embodiments, a CRISPR/Cas effector protein is a nuclease. In some embodiments, a CRISPR/Cas effector protein is a nickase. In some embodiments, a CRISPR/Cas effector protein is engineered (e.g., made by hand of man). In some embodiments, a CRISPR/Cas effector protein is a variant CRISPR/Cas effector protein.

The term “CRISPR/Cas nuclease” as used herein refer to any CRISPR/Cas protein with DNA nuclease activity, e.g., a Cas12a protein that exhibits specific association (or “targeting”) to a DNA target site, e.g., within a genomic sequence in a cell in the presence of a guide molecule. The strategies, systems, and methods disclosed herein can use any combination of CRISPR/Cas nuclease disclosed herein.

The term “CRISPR/Cas nickase” as used herein refer to any CRISPR/Cas protein with DNA nickase activity, e.g., a Cas12a protein that exhibits specific association (or “targeting”) to a DNA target site, e.g., within a genomic sequence in a cell in the presence of a guide molecule. The strategies, systems, and methods disclosed herein can use any combination of CRISPR/Cas nickase(s) disclosed herein.

The term “fuse.” or “fused” refers to the covalent linkage between two polypeptides in a fusion protein. The polypeptides may be fused via a peptide bond, either directly to each other or via a linker. The term “fusion protein” refers to a protein having at least two polypeptides covalently linked, either directly or via a linker (e.g., an amino acid linker). The polypeptides forming a fusion protein may be linked C-terminus to N-terminus, C-terminus to C-terminus, N-terminus to N-terminus, or N-terminus to C-terminus. The polypeptides of the fusion protein may be in any order and may include more than one of either or both of the constituent polypeptides. The term “fusion protein” encompasses conservatively modified variants, polymorphic variants, alleles, mutants, subsequences, interspecies homologs, and fragments of the poly peptides that make up the fusion protein. A fusion protein may be a protein developed from a fusion gene that is created through adjoining of two or more genes originally coding for separate proteins. Translation of this fusion gene may result in a single or multiple polypeptides with functional properties derived from each of the original proteins.

The term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest: in some embodiments, control elements act in trans to the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “nuclease” as used herein refers to any protein that catalyzes the cleavage of phosphodiester bonds. In some embodiments the nuclease is a DNA nuclease. In some embodiments the nuclease is a “nickase” which causes a single-strand break when it cleaves double-stranded DNA, e.g., genomic DNA in a cell. In some embodiments the nuclease causes a double-strand break when it cleaves double-stranded DNA, e.g., genomic DNA in a cell. In some embodiments the nuclease binds a specific target site within the double-stranded DNA that overlaps with or is adjacent to the location of the resulting break. In some embodiments, the nuclease causes a double-strand break that contains overhangs ranging from 0 (blunt ends) to 22 nucleotides in both 3′ and 5′ orientations. As discussed herein, CRISPR/Cas nucleases are exemplary nucleases that can be used in accordance with the strategies, systems, and methods of the present disclosure.

The term “nucleic acid” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside): in some embodiments, “nucleic acid” refers to an oligonucleotide chain comprising individual nucleic acid residues. In some embodiments, a “nucleic acid” is or comprises RNA: in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded: in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

The terms “orthologue” (also referred to as “ortholog” herein) and “homologue” (also referred to as “homolog” herein) are known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 22 (4): 359-66 (2013)). See also Shmakov et al. (2015) for application in the field of CRISPR/Cas loci. Homologous proteins may but need not be structurally related, or are only partially structurally related.

The term “endogenous,” as used herein in the context of nucleic acids refers to a native nucleic acid (e.g., a gene, a protein coding sequence) in its natural location, e.g., within the genome of a cell.

The term “exogenous,” as used herein in the context of nucleic acids refers to a nucleic acid (whether native or non-native) that has been artificially introduced into a man-made construct (e.g., a knock-in cassette, or a donor template) or into the genome of a cell using, for example, gene editing or genetic engineering techniques, e.g., HDR based integration techniques.

The term “guide molecule” or “guide RNA” or “gRNA” or “gRNA molecule” when used in reference to a CRISPR/Cas system is any nucleic acid that promotes the specific association (or “targeting”) of a CRISPR/Cas effector protein, e.g., a Cas12 effector protein to a DNA target site such as within a genomic sequence in a cell. While guide molecules are typically RNA molecules it is well known in the art that chemically modified RNA molecules including DNA/RNA hybrid molecules can be used as guide molecules.

The term “linker” is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (see e.g., Holliger et al., Proc. Natl. Acad. Sci. USA 90:6444-6448 (1993); Poljak et al., Structure 2:1121-1123 (1994)).

The term “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleared through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the addition of a series of adenosines to the 3′ end of the cleaved mRNA.

The term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof.

The term “polynucleotide” (including, but not limited to “nucleotide sequence”, “nucleic acid”, “nucleic acid molecule”, “nucleic acid sequence”, and “oligonucleotide”) as used herein refer to a series of nucleotide bases (also called “nucleotides”) in DNA and RNA, and mean any chain of two or more nucleotides. In some embodiments, polynucleotides, nucleotide sequences, nucleic acids, etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids containing modified bases.

Conventional IUPAC notation is used in nucleotide sequences presented herein, as shown in Table 1, below (see also Cornish-Bowden, Nucleic Acids Res. 13 (9): 3021-30 (1985), incorporated by reference herein). It should be noted, however, that “T” denotes “Thymine or Uracil” in those instances where a sequence may be encoded by either DNA or RNA, for example in certain CRISPR/Cas guide molecules.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “prevent,” “preventing,” and “prevention” as used herein refer to the prevention of a disease in a mammal, e.g., in a human, including (a) avoiding or precluding the disease; (b) affecting the predisposition toward the disease; or (c) preventing or delaying the onset of at least one symptom of the disease.

As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell: polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

The term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference: in some embodiments, a reference is a positive control reference.

The term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In some embodiments of this disclosure, the non-human animal subject is livestock, e.g., a cow, a horse, a sheep, a goat, etc. In some embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible subject prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

The term “variant” as used herein refers to an entity such as a polypeptide or polynucleotide that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As used herein, the terms “functional variant” refer to a variant that confers the same function as the reference entity. It is to be understood that a functional variant need not be functionally equivalent to the reference entity as long as it confers the same function as the reference entity.

### CRISPR Cas Effector Systems

CRISPR/Cas effector systems according to the present disclosure comprise, but are not limited to, naturally-occurring Class 2 CRISPR effector proteins such as Cas12a (Cpf1), as well as other Cas12 effector proteins and effector proteins derived or obtained therefrom. In functional terms, CRISPR/Cas effector systems are defined as comprising a CRISPR/Cas effector protein that: (A) interact with (e.g., complex with) a gRNA molecule; and (B) together with the gRNA molecule, associate with, and optionally alter, cleave or modify, a target region of a DNA that includes (1) a sequence complementary to the targeting domain of the gRNA and, optionally, (2) an additional sequence referred to as a “protospacer adjacent motif,” or “PAM,” which is described in greater detail below. As the following examples will illustrate, CRISPR/Cas effector proteins can be defined, in broad terms, by their PAM specificity and cleavage activity, even though variations may exist between individual CRISPR/Cas effector proteins that share the same PAM specificity or cleavage activity. Skilled artisans will appreciate that some aspects of the present disclosure relate to systems and methods that can be implemented using any suitable CRISPR/Cas effector proteins having a certain PAM specificity and/or cleavage activity. For this reason, unless otherwise specified, the term CRISPR/Cas effector proteins should be understood as a generic term, and not limited to any species (e.g., Acidaminococcus sp. vs. Lachnospiraceae bacterium) or variation (e.g., full-length vs. truncated or split: naturally-occurring PAM specificity vs. engineered PAM specificity, etc.) of CRISPR/Cas effector proteins.

In general, a CRISPR/Cas effector protein can be delivered to the cell as a protein or a nucleic acid encoding the protein, e.g., a DNA molecule or mRNA molecule. The protein or nucleic acid can be combined with other delivery agents, e.g., lipids or polymers in a lipid or polymer nanoparticle and targeting agents such as antibodies or other binding agents with specificity for the cell. The DNA molecule can be a nucleic acid vector, such as a viral genome or circular double-stranded DNA, e.g., a plasmid. Nucleic acid vectors encoding a CRISPR/Cas effector protein can include other coding or non-coding elements. For example, a CRISPR/Cas effector protein can be delivered as part of a viral genome (e.g., in an AAV, adenoviral or lentiviral genome) that includes certain genomic backbone elements (e.g., inverted terminal repeats, in the case of an AAV genome).

The CRISPR/Cas effector proteins described herein have activities and properties that can be useful in a variety of applications, but the skilled artisan will appreciate that CRISPR/Cas effector proteins can also be modified in certain instances, to alter cleavage activity, PAM specificity, or other structural or functional features.

For example, a CRISPR/Cas effector system may comprise a nuclease, nickase, inactive or dead CRISPR/Cas effector protein, or base editor as described herein. In some embodiments, a nuclease may nick both a target strand of a DNA sequence and a non-target strand of a DNA sequence to create a double-strand break to create indels in the genome of a cell comprising the DNA sequence as described herein. In some embodiments, a CRISPR/Cas effector system comprises a nickase. In some embodiments, a CRISPR/Cas effector system comprises a CRISPR/Cas effector protein with no nuclease/nickase/cutting activity which simply binds to a target nucleic acid sequence e.g., an inactive or dead Cas12a effector protein or dCas12a effector protein. It is contemplated that the nuclease, nickase, inactive or dead CRISPR/Cas effector proteins described herein can be delivered to a cell in vitro, in vivo, or ex vivo.

In some embodiments, the present disclosure describes CRISPR/Cas effector protein fusions for improved base editing activity (“base editors”). In some embodiments, base editors comprise a CRISPR/Cas effector protein fused to a deaminase that nicks only a target strand of a target nucleic sequence and then a deaminase makes either an I or U base edit which after repair leads to either a permanent C to T or an A to G change in the genome of a cell as described herein. In some embodiments, base editors comprise a dead CRISPR/Cas (e.g., dCas12a) effector protein having one or more mutations as described herein. In some embodiments, base editors comprise a wild-type CRISPR/Cas effector protein having one or more mutations as described herein. In some embodiments, base editors comprise a CRISPR/Cas effector protein that is a nickase as described herein. In some embodiments, base editors comprise a CRISPR/Cas effector protein that is a nickase having one or more mutations as described herein. In some embodiments, base editors can be used for a DNA target nucleic sequence that requires a CRISPR/Cas effector protein with a T-rich PAM, e.g., those within introns to correct splicing-defect mutations. In some embodiments, a Cas12a effector protein described herein may be fused to a deaminase or catalytic domain thereof to produce a base editor (BE), e.g., as described by PCT Publication Nos. WO 2018/176009A1, WO 2018/213708A1, WO 2018/213726A1, WO 2019/041296A1, WO 2019/126762A2, WO 2019/120310A1, WO 2019/161783A1, WO 2021/016086A1, WO 2021/087246A1, WO 2021/123397A1, or WO 2021/155109A1, the contents of each of which is hereby incorporated herein by reference in its entirety. It is contemplated that the base editors described herein can be delivered to a cell in vitro, in vivo, or ex vivo.

CRISPR/Cas effector proteins may also optionally include a tag, such as, but not limited to, a nuclear localization signal, to facilitate movement of the CRISPR/Cas effector protein into the nucleus. In some embodiments, the CRISPR/Cas effector protein can incorporate C- and/or N-terminal nuclear localization signals. Nuclear localization sequences are known in the art.

In some embodiments, CRISPR/Cas effector systems and methods of their use are described in US Publication No. 2019/0062735 A1, the disclosure of which are incorporated by reference herein in its entirety.

### Cas12a Effector Proteins and Variants Thereof

The present disclosure describes the use of Cas12a effector proteins, derived from a Cas12a locus denoted as subtype V-A, and variants thereof. Such effector proteins are also referred to herein as Cas12a effector proteins. Presently, the subtype V-A loci encompasses Cas1, Cas2, a distinct gene denoted Cas12a and a CRISPR array. Cpf1 (CRISPR-associated protein Cpf1, subtype PREFRAN) (Cas12a) is a large protein (about 1300 amino acids) that contains a RuvC-like nuclease domain homologous to the corresponding domain of Cas9 along with a counterpart to the characteristic arginine-rich cluster of Cas9. However, Cas12a lacks the HNH nuclease domain that is present in all Cas9 proteins, and the RuvC-like domain is contiguous in the Cas12a sequence, in contrast to Cas9 where it contains long inserts including the HNH domain. Accordingly, in particular embodiments, a Cas12a effector protein comprises only a RuvC-like nuclease domain.

A crystal structure of Acidaminococcus sp. Cas12a in complex with crRNA and a dsDNA target including a TTTN PAM sequence has been solved by Yamano et al., Cell 165 (4): 949-962 (2016). Cas12a has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and —III) and a bridge helix (BH) domain. However, the Cas12a REC lobe lacks an HNH domain, and includes other domains that also lack similarity to known protein structures: a structurally unique PAM-interacting (PI) domain, three Wedge (WED) domains (WED-I, -II and —III), and a nuclease (Nuc) domain.

In some embodiments, a Cas12a effector protein is derived from an organism from the genus of Eubacterium. In some embodiments, the CRISPR effector protein is a Cas12a effector protein derived from an organism from the bacterial species of Eubacterium rectale (ErCas12a, e.g., MAD7). In some embodiments, the amino acid sequence of a Cas12a effector protein corresponds to NCBI Reference Sequence WP_055225123.1, NCBI Reference Sequence WP_055237260.1, NCBI Reference Sequence WP 055272206.1, or GenBank ID OLA16049.1. In some embodiments, the homologue or orthologue of Cas12a as referred to herein has a sequence homology or identity of at least 60%, at least 70%, at least 80%, at least 85%, at least 90%, at least 95%, at least 97%, at least 98%, or at least 99% with one or more of the Cas12a sequences disclosed herein, e.g., one or more of the ErCas12a sequences disclosed herein. In further embodiments, the homologue or orthologue of Cas12a as referred to herein has a sequence identity of at least 80%, at least 85%, at least 90%, at least 95%, at least 97%, at least 98%, at least 99% with a wild-type ErCas12a. In some embodiments, a Cas12a effector protein comprises an amino acid sequence of SEQ ID NO: 15. In some embodiments, a Cas12a effector protein comprises an amino acid sequence of SEQ ID NO: 16. In some embodiments, a Cas12a effector protein has a sequence homology or sequence identity of at least 60%, at least 70%, at least 80%, at least 85%, at least 90%, at least 95%, at least 97%, at least 98%, or at least 99% with NCBI Reference Sequence WP_055225123.1, NCBI Reference Sequence WP_055237260.1, NCBI Reference Sequence WP 055272206.1, GenBank ID OLA16049.1, SEQ ID NO: 15, or SEQ ID NO: 16. A skilled person will understand that this includes truncated forms of a Cas12a effector protein whereby the sequence identity is determined over the length of the truncated form. In some embodiments, the ErCas12a effector protein recognizes the PAM sequence of TTTN or CTTN.

In some embodiments, a Cas12a effector protein may be from an organism of a genus which includes, but is not limited to Acidaminococcus sp. Lachnospiraceae bacterium, Francisella tularensis subsp. Novicida, Moraxella bovoculi, or Eubacterium rectale. In some embodiments, a Cas12a effector protein may be an organism of a species which includes, but is not limited to Acidaminococcus sp. BV316 (AsCas12a); Lachnospiraceae bacterium ND2006 (LbCas12a); or Lachnospiraceae bacterium MA2020 (Lb2Cas12a). In some embodiments, the homologue or orthologue of Cas12a as referred to herein has a sequence homology or identity of at least 80%, more preferably at least 85%, even more preferably at least 90%, such as for instance at least 95%, such as for instance at least 97%, such as for instance at least 98%, such as for instance at least 99% with one or more of the Cas12a sequences disclosed herein. In further embodiments, the homologue or orthologue of Cas12a as referred to herein has a sequence identity of at least 80%, more preferably at least 85%, even more preferably at least 90%, such as for instance at least 95%, such as for instance at least 97%, such as for instance at least 98%, such as for instance at least 99% with a wild-type ErCas12a, FnCas12a, AsCas12a, LbCas12a, Lb2Cas12a, MbCas12a, or MG29-1. In some embodiments, a Cas12a effector protein comprises an amino acid sequence of SEQ ID NO: 1. In some embodiments, a Cas12a effector protein comprises an amino acid sequence of SEQ ID NO: 2. In some embodiments, a Cas12a effector protein comprises an amino acid sequence of SEQ ID NO: 3. In some embodiments, a Cas12a effector protein comprises an amino acid sequence of SEQ ID NO: 4. In some embodiments, a Cas12a effector protein comprises an amino acid sequence of SEQ ID NO: 5. In some embodiments, a Cas12a effector protein comprises an amino acid sequence of SEQ ID NO: 14. In some embodiments, a Cas12a effector protein comprises an amino acid sequence of SEQ ID NO: 15.

In some embodiments, a Cas12a effector protein has a sequence homology or identity of at least 60%, more particularly at least 70%, at least 80%, more preferably at least 85%, even more preferably at least 90%, at least 95%, at least 97%, at least 98%, or at least 99%, with ErCas12a, AsCas12a, FnCas12a, LbCas12a, Lb2Cas12a, MbCas12a, or MG29-1. In some embodiments, a Cas12a effector protein as referred to herein has a sequence identity of at least 60%, at least 70%, at least 80%, at least 85%, at least 90%, at least 95%, at least 97%, at least 98%, or at least 99%, with a wild-type ErCas12a, AsCas12a, FnCas12a, LbCas12a, Lb2Cas12a, MbCas12a, or MG29-1. In some embodiments, a Cas12a effector protein has less than 60% sequence identity with AsCas12a. In some embodiments, a Cas12a effector protein has less than 60% sequence identity with ErCas12a. In some embodiments, a Cas12a effector protein has less than 60% sequence identity with FnCas12a. In some embodiments, a Cas12a effector protein has less than 60% sequence identity with LbCas12a. In some embodiments, a Cas12a effector protein has less than 60% sequence identity with Lb2Cas12a. In some embodiments, a Cas12a effector protein has less than 60% sequence identity with MbCas12a. In some embodiments, a Cas12a effector protein has less than 60% sequence identity with MG29-1. A skilled person will understand that this includes truncated forms of a Cas12a effector protein whereby the sequence identity is determined over the length of the truncated form.

In some embodiments, a homologue or orthologue of Cas12a as referred to herein has a sequence homology or identity of at least 80%, at least 85%, at least 90%, at least 95%, at least 97%, at least 98%, or at least 99% with Cas12a. In further embodiments, the homologue or orthologue of Cas12a as referred to herein has a sequence identity of at least 80%, at least 85%, at least 90%, at least 95%, at least 97%, at least 98%, or at least 99% with a wild-type Cas12a. Where the Cas12a has one or more mutations (mutated), the homologue or orthologue of the Cas12a as referred to herein has a sequence identity of at least 80%, at least 85%, at least 90%, at least 95%, at least 97%, at least 98%, or at least 99% with the mutated Cas12a.

Cas12a effector proteins may also refer to Cas12a nucleases, Cas12a nickases, and/or dead Cas12a effector proteins, and related variants thereof as described herein. In some embodiments, Cas12a effector proteins are fused to one or more heterologous protein domains (“fusion proteins”) for base editing as described herein.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular CRISPR/Cas effector proteins, but it should be understood that the CRISPR/Cas effector proteins used may be modified in ways that do not alter their operating principles. Such modifications are within the scope of the present disclosure.

Turning first to modifications that alter cleavage activity of Cas12a effector proteins, the present disclosure describes substitutions (or mutations) that reduce or eliminate activity of domains within the NUC lobe. In general, mutations that reduce or eliminate activity in nuclease domains result in CRISPR/Cas effector proteins with nickase activity, but it should be noted that the type of nickase activity varies depending on which domain is inactivated. For example, exemplary mutations at positions corresponding to K1000, S1001, e.g., K1000G, S1001G in AsCas12a may be made as described by PCT Publication No. WO 2019/233990A1, the entire contents of which are incorporated herein by reference.

As another example, exemplary mutations are included that alter the PAM specificity of ErCas12a variants, e.g., those at positions K535, K594, e.g., K535R, K594L, e.g., K535R/N539S, K535R/N539S/K594L/E730Q, K535R/N539S and K535R/N539S/K594L/E730Q as described in WO 2020/086475A1 or those at positions K169, N264, D529, K535, N539, and K594, which are corresponding to exemplary mutations at positions K177, N272, D537, K543, N547, K602, e.g., K177R, N272A, D537R, K543V, K543R, N547R. K602R as described in WO 2021/074191A1, the entire contents of each of which are incorporated herein by reference. However, it is noted that the combination of substitutions described by the present disclosure result in unexpectedly increased activity compared to variants described by the art.

In some embodiments, a Cas12a effector protein is a Cas12a variant comprising 1, 2, 3, 4, 5, 6, or 7 of the amino acid substitutions corresponding to substitutions at M537, F870, S186, R301, T315, Q1014, and E174 in AsCas12a. In some embodiments, a Cas12a effector protein is a Cas12a variant comprising 1, 2, 3, 4, 5, 6, or 7 of the amino acid substitutions corresponding to substitutions at M537R, F870L, S186K, R301K, T315R. Q1014R, and E174R in AsCas12a. In some embodiments, a Cas12a variant is a nickase comprising one or more additional amino acid substitutions (e.g., at positions corresponding to substitutions at K1000, S1001, e.g., K1000G, S1001G in AsCas12a). In some embodiments, a Cas12a effector protein is a Cas12a nickase comprising one or more additional amino acid substitutions corresponding to substitutions at R1226 in AsCas12a (e.g., at a position corresponding to a substitution at R1226, e.g., R1226A in AsCas12a).

In some embodiments, an AsCas12a effector protein comprises amino acid substitutions E174, S542, and K548 in AsCas12a. In some embodiments, a Cas12a variant is a nickase comprising one or more additional amino acid substitutions (e.g., at position corresponding to a substitution at R1226, e.g., R1226A in AsCas12a).

In some embodiments, a Cas12a effector protein is an ErCas12a variant comprising 1, 2, 3, 4, 5, 6, or 7 of the amino acid substitutions corresponding to substitutions at M537, F870, S186, R301, T315, Q1014, and E174 in AsCas12a. In some embodiments, a Cas12a effector protein is an ErCas12a variant comprising 1, 2, 3, 4, 5, 6, or 7 of the amino acid substitutions corresponding to substitutions at M537R, F870L, S186K. R301K, T315R, Q1014R, and E174R in AsCas12a. In some embodiments, an ErCas12a variant is a nickase comprising one or more additional amino acid substitutions (e.g., at positions corresponding to K1000, S1001, e.g., K1000G, S1001G in AsCas12a). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R, N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, a Cas12a effector protein is an ErCas12a variant comprising 1 or 2 amino acid substitutions at positions selected from I524 and F840. In some embodiments, a Cas12a effector protein is an ErCas12a variant comprising 1 or 2 of the amino acid substitutions selected from I524R and F840L. In some embodiments, an ErCas12a variant is a nickase comprising one or more additional amino acid substitutions (e.g., at positions K969, K970, e.g., K969G, K970G). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R, N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, a Cas12a effector protein is an ErCas12a variant comprising 1, 2, 3, 4, 5, 6, or 7 amino acid substitutions selected from substitutions at I524, F840, S181, T292, K982, K169, and D1055. In some embodiments, a Cas12a effector protein is an ErCas12a variant comprising 1, 2, 3, 4, 5, 6 or 7 of the amino acid substitutions selected from substitutions at I524R, F840L, S181K, T292R, K982R, K169R, and D1055Y. In some embodiments, an ErCas12a variant is a nickase comprising one or more additional amino acid substitutions (e.g., at positions K969, K970, e.g., K969G, K970G). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R, N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, a Cas12a effector protein is an ErCas12a variant comprising 1, 2, 3, 4 or 5 amino acid substitutions at positions selected from I524, F840, S181, T292, and K982. In some embodiments, a Cas12a effector protein is an ErCas12a variant comprising 1, 2, 3, 4, or 5 of the amino acid substitutions selected from I524R, F840L, S181K, T292R, and K982R. In some embodiments, an ErCas12a variant is a nickase comprising one or more additional amino acid substitutions (e.g., at positions K969, K970, e.g., K969G, K970G). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R, N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, a Cas12a effector protein is an ErCas12a variant comprising 1, 2 or 3 amino acid substitutions at positions selected from I524, F840, and K169. In some embodiments, a Cas12a effector protein is an ErCas12a variant comprising 1, 2 or 3 of the amino acid substitutions selected from I524R, F840L, and K169R. In some embodiments, an ErCas12a variant is a nickase comprising one or more additional amino acid substitutions (e.g., at positions K969, K970, e.g., K969G, K970G). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R, N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, an ErCas12a effector protein comprises amino acid substitutions I524R and F840L. In some embodiments, an ErCas12a variant is a nickase comprising one or more additional amino acid substitutions (e.g., at positions K969, K970, e.g., K969G, K970G). In some embodiments, an ErCas12a variant is an ErCas12a PAM variant comprising one or more additional amino acid substitutions (e.g., at positions K535, K594, e.g., K535R, K594L, or at positions K169, N264, D529, K535, N539, and K594, e.g., K169R. N264A, D529R, K535V, K535R, N539R, and K594R). In some embodiments, an ErCas12a effector protein is an ErCas12a nickase comprising one or more additional amino acid substitutions (e.g., at position R1173, e.g., R1173A).

In some embodiments, an ErCas12a effector protein comprises amino acid substitutions K169R, D529R, and K535R. In some embodiments, an ErCas12a effector protein is an ErCas12a nickase comprising one or more additional amino acid substitutions (e.g., at position R1173, e.g., R1173A).

In some embodiments, a Cas12a effector protein is an FnCas12a variant comprising 1 or 2 amino acid substitutions at positions selected from N602 and F879. In some embodiments, a Cas12a effector protein is an FnCas12a variant comprising 1 or 2 of the amino acid substitutions selected from N602R and F879L. In some embodiments, an FnCas12a variant is a nickase comprising one or more additional amino acid substitutions (e.g., at positions K1013, R1014, e.g., K1013G, R1014G). In some embodiments, an FnCas12a variant is a nickase comprising one or more additional amino acid substitutions (e.g., at position R1218, e.g., R1218A).

In some embodiments, a Cas12a effector protein is an FnCas12a variant comprising 1, 2, 3, 4 or 5 amino acid substitutions at positions selected from N602, F879, P196, S334, and K1026. In some embodiments, a Cas12a effector protein is an FnCas12a variant comprising 1, 2, 3, 4, or 5 of the amino acid substitutions selected from N602R, F879L, P196K, S334R, and K1026R. In some embodiments, an FnCas12a variant is a nickase comprising one or more additional amino acid substitutions (e.g., at positions K1013, R1014, e.g., K1013G, R1014G). In some embodiments, an FnCas12a variant is a nickase comprising one or more additional amino acid substitutions (e.g., at position R1218, e.g., R1218A).

In some embodiments, a Cas12a effector protein is an FnCas12a variant comprising 1, 2 or 3 amino acid substitutions at positions selected from N602, F879, and E184. In some embodiments, a Cas12a effector protein is an FnCas12a variant comprising 1, 2 or 3 of the amino acid substitutions selected from N602R, F879L, and E184R. In some embodiments, an FnCas12a variant is a nickase comprising one or more additional amino acid substitutions (e.g., at positions K1013, R1014, e.g., K1013G, R1014G). In some embodiments, an FnCas12a variant is a nickase comprising one or more additional amino acid substitutions (e.g., at position R1218, e.g., R1218A).

In some embodiments, an FnCas12a effector protein comprises amino acid substitutions N602R and F879L. In some embodiments, an FnCas12a variant is a nickase comprising one or more additional amino acid substitutions (e.g., at positions K1013, R1014, e.g., K1013G, R1014G). In some embodiments, an FnCas12a variant is a nickase comprising one or more additional amino acid substitutions (e.g., at position R1218, e.g., R1218A).

In some embodiments, a FnCas12a effector protein comprises amino acid substitutions E184R, N607R, and K613R. In some embodiments, a FnCas12a effector protein is a FnCas12a nickase comprising one or more additional amino acid substitutions (e.g., at position R1218, e.g., R1218A).

In some embodiments, a Cas12a effector protein amino acid sequence comprises an amino acid sequence having at least about 90%, 95%, 97%, 98%, 99% or 100% identity to an FnCas12a amino acid sequence described herein.

In some embodiments, a Cas12a effector protein is an Lb2Cas12a variant comprising 1 or 2 amino acid substitutions at positions selected from R507 and T778. In some embodiments, a Cas12a effector protein is an Lb2Cas12a variant comprising the amino acid substitution of T778L. In some embodiments, an Lb2Cas12a variant is a nickase comprising one or more additional amino acid substitutions (e.g., at positions K913, R914, e.g., K913G, R914G). In some embodiments, an Lb2Cas12a variant is a nickase comprising one or more additional amino acid substitutions (e.g., at a position corresponding to a substitution at R1124, e.g., R1124A).

In some embodiments, a Cas12a effector protein is an Lb2Cas12a variant comprising 1, 2, 3, 4 or 5 amino acid substitutions at positions selected from R507, T778, S167, E271, and K926. In some embodiments, a Cas12a effector protein is an Lb2Cas12a variant comprising 1, 2, 3, or 4 of the amino acid substitutions selected from T778L, S167K, E271R, and K926R. In some embodiments, an Lb2Cas12a variant is a nickase comprising one or more additional amino acid substitutions (e.g., at positions K913, R914, e.g., K913G, R914G). In some embodiments, an Lb2Cas12a variant is a nickase comprising one or more additional amino acid substitutions (e.g., at position R1124, e.g., R1124A).

In some embodiments, a Cas12a effector protein is an Lb2Cas12a variant comprising 1, 2 or 3 amino acid substitutions at positions selected from R507, T778, and K155. In some embodiments, a Cas12a effector protein is an Lb2Cas12a variant comprising 1 or 2 of the amino acid substitutions selected from T778L and K155R. In some embodiments, an Lb2Cas12a variant is a nickase comprising one or more additional amino acid substitutions (e.g., at positions K913, R914, e.g., K913G, R914G). In some embodiments, an Lb2Cas12a variant is a nickase comprising one or more additional amino acid substitutions (e.g., at position R1124, e.g., R1124A).

In some embodiments, an Lb2Cas12a effector protein comprises amino acid substitution T778L. In some embodiments, an Lb2Cas12a variant is a nickase comprising one or more additional amino acid substitutions (e.g., at positions K913, R914, e.g., K913G, R914G). In some embodiments, an Lb2Cas12a variant is a nickase comprising one or more additional amino acid substitutions (e.g., at position R1124, e.g., R1124A).

In some embodiments, a Lb2Cas12a effector protein comprises amino acid substitutions K155R, N512R, and K518R. In some embodiments, an Lb2Cas12a effector protein is a Lb2Cas12a nickase comprising one or more additional amino acid substitutions (e.g., at position R1124, e.g., R1124A).

In some embodiments, a Cas12a effector protein amino acid sequence comprises an amino acid sequence having at least about 90%, 95%, 97%, 98%, 99% or 100% identity to an Lb2Cas12a amino acid sequence described herein.

In some embodiments, a Cas12a effector protein is an LbCas12a variant comprising 1 or 2 amino acid substitutions at positions selected from N527 and E795. In some embodiments, a Cas12a effector protein is an LbCas12a variant comprising 1 or 2 of the amino acid substitutions selected from N527R and E795L. In some embodiments, an LbCas12a variant is a nickase comprising one or more additional amino acid substitutions (e.g., at positions K932, N933, e.g., K932G, N933G). In some embodiments, an LbCas12a variant is a nickase comprising one or more additional amino acid substitutions (e.g., at position R1138, e.g., R1138A).

In some embodiments, a Cas12a effector protein is an LbCas12a variant comprising 1, 2, 3, 4 or 5 amino acid substitutions at positions selected from N527, E795, S168, S286, and K945. In some embodiments, a Cas12a effector protein is an LbCas12a variant comprising 1, 2, 3, 4, or 5 of the amino acid substitutions selected from N527R, E795L, S168K, S286R, and K945R. In some embodiments, an LbCas12a variant is a nickase comprising one or more additional amino acid substitutions (e.g., at positions K932, N933, e.g., K932G, N933G). In some embodiments, an LbCas12a variant is a nickase comprising one or more additional amino acid substitutions (e.g., at position R1138, e.g., R1138A).

In some embodiments, a Cas12a effector protein is an LbCas12a variant comprising 1, 2 or 3 amino acid substitutions at positions selected from N527, E795, and D156. In some embodiments, a Cas12a effector protein is an LbCas12a variant comprising 1, 2 or 3 of the amino acid substitutions selected from N527R, E795L, and D156R. In some embodiments, an LbCas12a variant is a nickase comprising one or more additional amino acid substitutions (e.g., at positions K932, N933, e.g., K932G, N933G). In some embodiments, an LbCas12a variant is a nickase comprising one or more additional amino acid substitutions (e.g., at position R1138, e.g., R1138A).

In some embodiments, an LbCas12a effector protein comprises amino acid substitutions N527R and E795L. In some embodiments, an LbCas12a variant is a nickase comprising one or more additional amino acid substitutions (e.g., at positions K932, N933, e.g., K932G, N933G). In some embodiments, an LbCas12a variant is a nickase comprising one or more additional amino acid substitutions (e.g., at position R1138, e.g., R1138A).

In some embodiments, an LbCas12a effector protein comprises amino acid substitutions D156R, G532R, and K538R. In some embodiments, an LbCas12a effector protein is an LbCas12a nickase comprising one or more additional amino acid substitutions (e.g., at position R1138, e.g., R1138A).

In some embodiments, a Cas12a effector protein amino acid sequence comprises an amino acid sequence having at least about 90%, 95%, 97%, 98%, 99% or 100% identity to an LbCas12a amino acid sequence described herein.

In some embodiments, a Cas12a effector protein is an MbCas12a variant comprising 1 or 2 amino acid substitutions at positions selected from N568 and M825. In some embodiments, a Cas12a effector protein is an MbCas12a variant comprising 1 or 2 of the amino acid substitutions selected from N568R and M825L. In some embodiments, an MbCas12a variant is a nickase comprising one or more additional amino acid substitutions (e.g., at positions K965, R966, e.g., K965G, R966G). In some embodiments, an MbCas12a variant is a nickase comprising one or more additional amino acid substitutions (e.g., at position R1171, e.g., R1171A).

In some embodiments, a Cas12a effector protein is an MbCas12a variant comprising 1, 2, 3, 4 or 5 amino acid substitutions at positions selected from N568, M825, H184, G292, and N978. In some embodiments, a Cas12a effector protein is an MbCas12a variant comprising 1, 2, 3, 4, or 5 of the amino acid substitutions selected from N568R, M825L, H184K, G292R, and N978R. In some embodiments, an MbCas12a variant is a nickase comprising one or more additional amino acid substitutions (e.g., at positions K965, R966, e.g., K965G, R966G). In some embodiments, an MbCas12a variant is a nickase comprising one or more additional amino acid substitutions (e.g., at position R1171, e.g., R1171A).

In some embodiments, a Cas12a effector protein is an MbCas12a variant comprising 1, 2 or 3 amino acid substitutions at positions selected from N568, M825, and D172. In some embodiments, a Cas12a effector protein is an MbCas12a variant comprising 1, 2 or 3 of the amino acid substitutions selected from N568R, M825L, and D172R. In some embodiments, an MbCas12a variant is a nickase comprising one or more additional amino acid substitutions (e.g., at positions K965, R966, e.g., K965G, R966G). In some embodiments, an MbCas12a variant is a nickase comprising one or more additional amino acid substitutions (e.g., at position R1171, e.g., R1171A).

In some embodiments, an MbCas12a effector protein comprises amino acid substitutions N568R and M825L. In some embodiments, an MbCas12a variant is a nickase comprising one or more additional amino acid substitutions (e.g., at positions K965, R966, e.g., K965G, R966G). In some embodiments, an MbCas12a variant is a nickase comprising one or more additional amino acid substitutions (e.g., at position R1171, e.g., R1171A).

In some embodiments, an MbCas12a effector protein comprises amino acid substitutions D172R, N563R, and K569R. In some embodiments, an MbCas12a effector protein is a MbCas12a nickase comprising one or more additional amino acid substitutions (e.g., at position R1218, e.g., R1218A).

In some embodiments, a Cas12a effector protein amino acid sequence comprises an amino acid sequence having at least about 90%, 95%, 97%, 98%, 99% or 100% identity to an MbCas12a amino acid sequence described herein.

In some embodiments, a Cas12a effector protein is an AsCas12a variant comprising 1, 2, 3, 4, 5, 6, 7, or 8 amino acid substitutions at positions selected from M537, F870, E174, S186, R301, T315, Q1014, and I1088. In some embodiments, a Cas12a effector protein is an AsCas12a variant comprising 1, 2, 3, 4, 5, 6, 7 or 8 of the amino acid substitutions selected from M537R, F870L, E174R, S186K, R301K, T315R, Q1014R, and I1088Y. In some embodiments, an AsCas12a variant is a nickase comprising one or more additional amino acid substitutions (e.g., at positions K1000, K1001, e.g., K1000G, K1001G). In some embodiments, an AsCas12a variant is a nickase comprising one or more additional amino acid substitutions (e.g., at position R1226, e.g., R1226A).

In some embodiments, a Cas12a effector protein is an AsCas12a variant comprising 1 or 2 amino acid substitutions at positions selected from K603 and I1088. In some embodiments, a Cas12a effector protein is an AsCas12a variant comprising the amino acid substitutions I1088Y. In some embodiments, an AsCas12a variant is a nickase comprising one or more additional amino acid substitutions (e.g., at position R1226, e.g., R1226A).

In some embodiments, an AsCas12a effector protein comprises amino acid substitutions E174R, S542R, and K548R. In some embodiments, an AsCas12a effector protein is an AsCas12a nickase comprising one or more additional amino acid substitutions (e.g., at position R1226, e.g., R1226A).

In some embodiments, a Cas12a effector protein amino acid sequence comprises an amino acid sequence having at least about 90%, 95%, 97%, 98%, 99% or 100% identity to an AsCas12a amino acid sequence described herein.

In some embodiments, a Cas12a effector protein is an MG29-1 variant comprising 1, 2, 3, 4, 5, 6, or 7 of the amino acid substitutions corresponding to substitutions at M537, F870, S186, R301, T315, Q1014, and E174 in AsCas12a. In some embodiments, a Cas12a effector protein is an MG29-1 variant comprising 1, 2, 3, 4, 5, 6, or 7 of the amino acid substitutions corresponding to M537R, F870L, S186K, R301K, T315R, Q1014R, and E174R in AsCas12a. In some embodiments, an MG29-1 variant is a nickase comprising one or more additional amino acid substitutions (e.g., at positions corresponding to K1000, S1001, e.g., K1000G, S1001G in AsCas12a). In some embodiments, an MG29-1 variant is a nickase comprising one or more additional amino acid substitutions (e.g., at position R1192, e.g., R1192A).

In some embodiments, a Cas12a effector protein is an MG29-1 variant comprising 1 or 2 amino acid substitutions at positions selected from A572 and F849. In some embodiments, a Cas12a effector protein is an MG29-1 variant comprising 1 or 2 of the amino acid substitutions selected from A572R and F849L. In some embodiments, an MG29-1 variant is a nickase comprising one or more additional amino acid substitutions (e.g., at positions K983, R984, e.g., K983G, R984G). In some embodiments, an MG29-1 variant is a nickase comprising one or more additional amino acid substitutions (e.g., at position R1192, e.g., R1192A).

In some embodiments, a Cas12a effector protein is an MG29-1 variant comprising 1, 2, 3, 4 or 5 amino acid substitutions at positions selected from A572, F849, S184, R292, T306, and K996. In some embodiments, a Cas12a effector protein is an MG29-1 variant comprising 1, 2, 3, 4, or 5 of the amino acid substitutions selected from A572R, F849L, S184K, R292K, T306R, and K996R. In some embodiments, an MG29-1 variant is a nickase comprising one or more additional amino acid substitutions (e.g., at positions K983, R984, e.g., K983G, R984G). In some embodiments, an MG29-1 variant is a nickase comprising one or more additional amino acid substitutions (e.g., at position R1192, e.g., R1192A).

In some embodiments, a Cas12a effector protein is an MG29-1 variant comprising 1, 2 or 3 amino acid substitutions at positions selected from A572, F849, and E172. In some embodiments, a Cas12a effector protein is an MG29-1 variant comprising 1, 2 or 3 of the amino acid substitutions selected from A572R, F849L, and E172R. In some embodiments, an MG29-1 variant is a nickase comprising one or more additional amino acid substitutions (e.g., at positions K983, R984, e.g., K983G, R984G). In some embodiments, an MG29-1 variant is a nickase comprising one or more additional amino acid substitutions (e.g., at position R1192, e.g., R1192A).

In some embodiments, a Cas12a effector protein comprises amino acid substitutions A572R and F849L. In some embodiments, an MG29-1 variant is a nickase comprising one or more additional amino acid substitutions (e.g., at positions K983, R984, e.g., K983G, R984G). In some embodiments, an MG29-1 variant is a nickase comprising one or more additional amino acid substitutions (e.g., at position R1192, e.g., R1192A).

In some embodiments, an MG29-1 effector protein comprises amino acid substitutions E172R, N577R, and K583R. In some embodiments, an MG29-1 effector protein is an MG29-1 nickase comprising one or more additional amino acid substitutions (e.g., at position R1192, e.g., R1192A).

In some embodiments, a Cas12a effector protein amino acid sequence comprises an amino acid sequence having at least about 90%, 95%, 97%, 98%, 99% or 100% identity to an MG29-1 amino acid sequence described herein.

Other suitable modifications of a Cas12a amino acid sequence are known to those of ordinary skill in the art. Some exemplary amino acid sequences of wild-type Cas12a (Cpf1) effector proteins and variants thereof are provided below:

Exemplary AsCas12a wild-type amino acid sequence
SEQ ID NO: 1
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary FnCas12a wild-type amino acid sequence
SEQ ID NO: 2
MSIYQEFVNKYSLSKTLRFELIPQGKTLENIKARGLILDDEKRAK
DYKKAKQIIDKYHQFFIEEILSSVCISEDLLQNYSDVYFKLKKSD
DDNLQKDFKSAKDTIKKQISEYIKDSEKFKNLFNQNLIDAKKGQE
SDLILWLKQSKDNGIELFKANSDITDIDEALEIIKSFKGWTTYFK
GFHENRKNVYSSNDIPTSIIYRIVDDNLPKFLENKAKYESLKDKA
PEAINYEQIKKDLAEELTFDIDYKTSEVNQRVFSLDEVFEIANFN
NYLNQSGITKFNTIIGGKFVNGENTKRKGINEYINLYSQQINDKT
LKKYKMSVLFKQILSDTESKSFVIDKLEDDSDVVTTMQSFYEQIA
AFKTVEEKSIKETLSLLFDDLKAQKLDLSKIYFKNDKSLTDLSQQ
VFDDYSVIGTAVLEYITQQIAPKNLDNPSKKEQELIAKKTEKAKY
LSLETIKLALEEFNKHRDIDKQCRFEEILANFAAIPMIFDEIAQN
KDNLAQISIKYQNQGKKDLLQASAEDDVKAIKDLLDQTNNLLHKL
KIFHISQSEDKANILDKDEHFYLVFEECYFELANIVPLYNKIRNY
ITQKPYSDEKFKLNFENSTLANGWDKNKEPDNTAILFIKDDKYYL
GVMNKKNNKIFDDKAIKENKGEGYKKIVYKLLPGANKMLPKVFFS
AKSIKFYNPSEDILRIRNHSTHTKNGSPQKGYEKFEFNIEDCRKF
IDFYKQSISKHPEWKDFGFRFSDTQRYNSIDEFYREVENQGYKLT
FENISESYIDSVVNQGKLYLFQIYNKDFSAYSKGRPNLHTLYWKA
LFDERNLQDVVYKLNGEAELFYRKQSIPKKITHPAKEAIANKNKD
NPKKESVFEYDLIKDKRFTEDKFFFHCPITINFKSSGANKFNDEI
NLLLKEKANDVHILSIDRGERHLAYYTLVDGKGNIIKQDTFNIIG
NDRMKTNYHDKLAAIEKDRDSARKDWKKINNIKEMKEGYLSQVVH
EIAKLVIEYNAIVVFEDLNFGFKRGRFKVEKQVYQKLEKMLIEKL
NYLVFKDNEFDKTGGVLRAYQLTAPFETFKKMGKQTGIIYYVPAG
FTSKICPVTGFVNQLYPKYESVSKSQEFFSKFDKICYNLDKGYFE
FSFDYKNFGDKAAKGKWTIASFGSRLINFRNSDKNHNWDTREVYP
TKELEKLLKDYSIEYGHGECIKAAICGESDKKFFAKLTSVLNTIL
QMRNSKTGTELDYLISPVADVNGNFFDSRQAPKNMPQDADANGAY
HIGLKGLMLLGRIKNNQEGKKLNLVIKNEEYFEFVQNRNN
Exemplary Lb2Cas12a wild-type amino acid sequence
SEQ ID NO: 3
MYYESLTKQYPVSKTIRNELIPIGKTLDNIRQNNILESDVKRKQN
YEHVKGILDEYHKQLINEALDNCTLPSLKIAAEIYLKNQKEVSDR
EDENKTQDLLRKEVVEKLKAHENFTKIGKKDILDLLEKLPSISED
DYNALESFRNFYTYFTSYNKVRENLYSDKEKSSTVAYRLINENFP
KFLDNVKSYRFVKTAGILADGLGEEEQDSLFIVETENKTLTQDGI
DTYNSQVGKINSSINLYNQKNQKANGFRKIPKMKMLYKQILSDRE
ESFIDEFQSDEVLIDNVESYGSVLIESLKSSKVSAFFDALRESKG
KNVYVKNDLAKTAMSNIVFENWRTFDDLLNQEYDLANENKKKDDK
YFEKRQKELKKNKSYSLEHLCNLSEDSCNLIENYIHQISDDIENI
IINNETFLRIVINEHDRSRKLAKNRKAVKAIKDFLDSIKVLEREL
KLINSSGQELEKDLIVYSAHEELLVELKQVDSLYNMTRNYLTKKP
FSTEKVKLNFNRSTLLNGWDRNKETDNLGVLLLKDGKYYLGIMNT
SANKAFVNPPVAKTEKVFKKVDYKLLPVPNQMLPKVFFAKSNIDF
YNPSSEIYSNYKKGTHKKGNMFSLEDCHNLIDFFKESISKHEDWS
KFGFKFSDTASYNDISEFYREVEKQGYKLTYTDIDETYINDLIER
NELYLFQIYNKDFSMYSKGKLNLHTLYFMMLFDQRNIDDVVYKLN
GEAEVFYRPASISEDELIIHKAGEEIKNKNPNRARTKETSTFSYD
IVKDKRYSKDKFTLHIPITMNFGVDEVKRENDAVNSAIRIDENVN
VIGIDRGERNLLYVVVIDSKGNILEQISLNSIINKEYDIETDYHA
LLDEREGGRDKARKDWNTVENIRDLKAGYLSQVVNVVAKLVLKYN
AIICLEDLNFGFKRGRQKVEKQVYQKFEKMLIDKLNYLVIDKSRE
QTSPKELGGALNALQLTSKFKSFKELGKQSGVIYYVPAYLTSKID
PTTGFANLFYMKCENVEKSKRFFDGFDFIRFNALENVFEFGFDYR
SFTQRACGINSKWTVCTNGERIIKYRNPDKNNMFDEKVVVVTDEM
KNLFEQYKIPYEDGRNVKDMIISNEEAEFYRRLYRLLQQTLQMRN
STSDGTRDYIISPVKNKREAYFNSELSDGSVPKDADANGAYNIAR
KGLWVLEQIRQKSEGEKINLAMTNAEWLEYAQTHLLKRPAATKKA
GQAKKKKGSYPYDVPDYAYPYDVPDYAYPYDVPDYA
Exemplary LbCas12a wild-type amino acid sequence
SEQ ID NO: 4
MSKLEKFTNCYSLSKTLRFKAIPVGKTQENIDNKRLLVEDEKRAE
DYKGVKKLLDRYYLSFINDVLHSIKLKNLNNYISLFRKKTRTEKE
NKELENLEINLRKEIAKAFKGNEGYKSLFKKDIIETILPEFLDDK
DEIALVNSFNGFTTAFTGFFDNRENMFSEEAKSTSIAFRCINENL
TRYISNMDIFEKVDAIFDKHEVQEIKEKILNSDYDVEDFFEGEFF
NFVLTQEGIDVYNAIIGGFVTESGEKIKGLNEYINLYNQKTKQKL
PKFKPLYKQVLSDRESLSFYGEGYTSDEEVLEVFRNTLNKNSEIF
SSIKKLEKLFKNFDEYSSAGIFVKNGPAISTISKDIFGEWNVIRD
KWNAEYDDIHLKKKAVVTEKYEDDRRKSFKKIGSFSLEQLQEYAD
ADLSVVEKLKEIIIQKVDEIYKVYGSSEKLFDADFVLEKSLKKND
AVVAIMKDLLDSVKSFENYIKAFFGEGKETNRDESFYGDFVLAYD
ILLKVDHIYDAIRNYVTQKPYSKDKFKLYFQNPQFMGGWDKDKET
DYRATILRYGSKYYLAIMDKKYAKCLQKIDKDDVNGNYEKINYKL
LPGPNKMLPKVFFSKKWMAYYNPSEDIQKIYKNGTFKKGDMFNLN
DCHKLIDFFKDSISRYPKWSNAYDFNFSETEKYKDIAGFYREVEE
QGYKVSFESASKKEVDKLVEEGKLYMFQIYNKDFSDKSHGTPNLH
TMYFKLLFDENNHGQIRLSGGAELFMRRASLKKEELVVHPANSPI
ANKNPDNPKKTTTLSYDVYKDKRFSEDQYELHIPIAINKCPKNIF
KINTEVRVLLKHDDNPYVIGIDRGERNLLYIVVVDGKGNIVEQYS
LNEIINNFNGIRIKTDYHSLLDKKEKERFEARQNWTSIENIKELK
AGYISQVVHKICELVEKYDAVIALEDLNSGFKNSRVKVEKQVYQK
FEKMLIDKLNYMVDKKSNPCATGGALKGYQITNKFESFKSMSTQN
GFIFYIPAWLTSKIDPSTGFVNLLKTKYTSIADSKKFISSFDRIM
YVPEEDLFEFALDYKNFSRTDADYIKKWKLYSYGNRIRIFRNPKK
NNVFDWEEVCLTSAYKELFNKYGINYQQGDIRALLCEQSDKAFYS
SFMALMSLMLQMRNSITGRTDVDFLISPVKNSDGIFYDSRNYEAQ
ENAILPKNADANGAYNIARKVLWAIGQFKKAEDEKLDKVKIAISN
KEWLEYAQTSVKHKRPAATKKAGQAKKKKGSYPYDVPDYAYPYDV
PDYAYPYDVPDYA
Exemplary MbCas12a wild-type amino acid sequence
SEQ ID NO: 5
MLFQDFTHLYPLSKTVRFELKPIGRTLEHIHAKNFLSQDETMADM
YQKVKVILDDYHRDFIADMMGEVKLTKLAEFYDVYLKFRKNPKDD
ELQKQLKDLQAVLRKESVKPIGNGGKYKAGHDRLFGAKLFKDGKE
LGDLAKFVIAQEGKSSPKLAHLAHFEKFSTYFTGFHDNRKNMYSD
EDKHTAIAYRLIHENLPRFIDNLQILTTIKQKHSALYDQIINELT
ASGLDVSLASHLDGYHKLLTQEGITAYNRIIGEVNGYTNKHNQIC
HKSERIAKLRPLHKQILSDGMGVSFLPSKFADDSEMCQAVNEFYR
HYADVFAKVQSLFDGFDDHQKDGIYVEHKNLNELSKQAFGDFALL
GRVLDGYYVDVVNPEFNERFAKAKTDNAKAKLTKEKDKFIKGVHS
LASLEQAIKHHTARHDDESVQAGKLGQYFKHGLAGVDNPIQKIHN
NHSTIKGFLERERPAGERALPKIKSGKNPEMTQLRQLKELLDNAL
NVAHFAKLLMTKTTLDNQDGNFYGEFGVLYDELAKIPTLYNKVRD
YLSQKPFSTEKYKLNFGNPTLLNGWDLNKEKDNFGVILQKDGCYY
LALLDKAHKKVFDNAPNTGKNVYQKMIYKLLPGPNKMLPRVFFAK
SNLDYYNPSAELLDKYAQGTHKKGDNFNLKDCHALIDFFKAGINK
HPEWQNFGFKFSPTSSYRDLSDFYREVEPQGYQVKFVDINADYID
ELVEQGQLYLFQIYNKDFSPKAHGKPNLHTLYFRALFSEDNLANP
IYKLNGEAQIFYRKASLGMNETTIHRAGEILENKNPDNPKERVFT
YDIIKDRRYTQDKFMLHVPITMNFGVQGMTIKEFNKKVNQSIRQY
DDVNVIGIDRGERHLLYLTVINSKGEILEQRSLNDITTASANGTQ
MTTPYHKILDKREIERLNARVGWGEIETIKELKSGYLSHVVHQVS
QLMLKYNAIVVLEDLNFGFKRGRFKVEKQIYQNFENALIKKLNHL
ELKDKADDEIGSYKNALQLTNNFTDLKNIGKQTGFLFYVPAWNTS
KIDPETGFVDLLKPRYENIAQSQAFFGKFDKICYNADKDYFEFHI
DYAKFTDKAKNSRQTWTICSHGDKRYVYDKTANQNKGATKGINVN
DELKSLFARYHINEKQPNLVMDICQNNDKEFHKSLMYLLKTLLAL
RYSNASSDEDFILSPVANDEGVFFNSALADDTQPQNADANGAYHI
ALKGLWLLNELKNSDDLNKVKLAIDNQTWLNFAQNR
Exemplary AsCas12a variant 1 amino acid sequence
SEQ ID NO: 6
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 3 amino acid sequence
SEQ ID NO: 7
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHKFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 4 amino acid sequence
SEQ ID NO: 8
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDIKTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQRFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 5 amino acid sequence
SEQ ID NO: 9
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDIKTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNK
SFILEEFKSDEEVIQSFCKYKTLLRNENVLETAEALFNELNSIDL
THIFISHKKLETISSALCDHWDTLRNALYERRISELTGKITKSAK
EKVQRSLKHEDINLQEIISAAGKELSEAFKQKTSEILSHAHAALD
QPLPTTLKKQEEKEILKSQLDSLLGLYHLLDWFAVDESNEVDPEF
SARLTGIKLEMEPSLSFYNKARNYATKKPYSVEKFKLNFQRPTLA
SGWDVNKEKNNGAILFVKNGLYYLGIMPKQKGRYKALSFEPTEKT
SEGFDKMYYDYFPDAAKMIPKCSTQLKAVTAHFQTHTTPILLSNN
FIEPLEITKEIYDLNNPEKEPKKFQTAYAKKTGDQKGYREALCKW
IDFTRDFLSKYTKTTSIDLSSLRPSSQYKDLGEYYAELNPLLYHI
SFQRIAEKEIMDAVETGKLYLFQIYNKDFAKGHHGKPNLHTLYWT
GLFSPENLAKTSIKLNGQAELFYRPKSRMKRMAHRLGEKMLNKKL
KDQKTPIPDTLYQELYDYVNHRLSHDLSDEARALLPNVITKEVSH
EIIKDRRFTSDKFLFHVPITLNYQAANSPSKFNQRVNAYLKEHPE
TPIIGIDRGERNLIYITVIDSTGKILEQRSLNTIQQFDYQKKLDN
REKERVAARQAWSVVGTIKDLKQGYLSQVIHEIVDLMIHYQAVVV
LENLNFGFKSKRTGIAEKAVYQRFEKMLIDKLNCLVLKDYPAEKV
GGVLNPYQLTDQFTSFAKMGTQSGFLFYVPAPYTSKIDPLTGFVD
PFVWKTIKNHESRKHFLEGFDFLHYDVKTGDFILHFKMNRNLSFQ
RGLPGFMPAWDIVFEKNETQFDAKGTPFIAGKRIVPVIENHRFTG
RYRDLYPANELIALLEEKGIVFRDGSNILPKLLENDDSHAIDTMV
ALIRSVLQMRNSNAATGEDYINSPVRDLNGVCFDSRFQNPEWPMD
ADANGAYHIALKGQLLLNHLKESKDLKLQNGISNQDWLAYIQELR
N
Exemplary AsCas12a variant 6 amino acid sequence
SEQ ID NO: 10
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDIKTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHKFIPLFKQILSDRNK
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQRFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 7 amino acid sequence
SEQ ID NO: 11
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFGGKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 8 amino acid sequence
SEQ ID NO: 12
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTYKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 9 amino acid sequence
SEQ ID NO: 13
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYRNRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 10 amino acid sequence
SEQ ID NO: 83
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYRNRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ARGWDVNREKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN
Exemplary AsCas12a variant 11 amino acid sequence
SEQ ID NO: 84
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYRNRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ARGWDVNREKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVFRDGSNILPKLLENDDSHAIDTM
VALIRSVLQMANSNAATGEDYINSPVRDLNGVCFDSRFQNPEWPM
DADANGAYHIALKGQLLLNHLKESKDLKLQNGISNQDWLAYIQEL
RN
Exemplary MG29-1 amino acid sequence
SEQ ID NO: 14
MFNNFIKKYSLQKTLRFELKPVGETADYIEDFKSEYLKDTVLKDE
QRAKDYQEIKTLIDDYHREYIEECLREPVDKKTGEILDFTQDLED
AFSYYQKLKENPTENRVGWEKEQESLRKKLVTSFVGNDGLFKKEF
ITRDLPEWLQKKGLWGEYKDTVENFKKFTTYFSGFHENRKNMYTA
EAQSTAIANRLMNDNLPKFFNNYLAYQTIKEKHPDLVFRLDDALL
QAAGVEHLDEAFQPRYFSRLFAQSGITAFNELIGGRTTENGEKIQ
GLNEQINLYRQQNPEKAKGFPRFMPLFKQILSDRETHSFLPDAFE
NDKELLQALRDYVDAATSEEGMISQLNKAMNQFVTADLKRVYIKS
AALTSLSQELFHFFGVISDAIAWYAEKRLSPKKAQESFLKQEVYA
IEELNQAVVGYIDQLEDQSELQQLLVDLPDPQKPVSSFILTHWQK
SQEPLQAVIAKVEPLFELEELSKNKRAPKHDKDQGGEGFQQVDAI
KNMLDAFMEVSHAIKPLYLVKGRKAIDMPDVDTGFYADFAEAYSA
YEQVTVSLYNKTRNHLSKKPFSKDKIKINFDAPTLLNGWDLNKES
DNKSIILRKDGNFYLAIMHPKHTKVFDCYSASEAAGKCYEKMNYK
LLSGANKMLPKVFFSKKGIETFSPPQEILDLYKNNEHKKGATFKL
ESCHKLIDFFKRNIPKYKVHPTDNFGWDVFGFHFSPTSSYGDLSG
FYREVEAQGYKLWFSDVSEAYINKCVEEGKLFLFQIYNKDFSPNS
TGKPNLHTLYWKGLFEPENLKDVVLKLNGEAEIFYRKHSIKHEDK
TIHRAKDPIANKNADNPKKQSVFDYDIIKDKRYTQDKFFFHVPIS
LNFKSQGVVRFNDKINGLLAAQDDVHVIGIDRGERHLLYYTVVNG
KGEVVEQGSLNQVATDQGYVVDYQQKLHAKEKERDQARKNWSTIE
NIKELKAGYLSQVVHKLAQLIVKHNAIVCLEDLNFGFKRGRFKVE
KQVYQKFEKALIDKLNYLVFKERGATQAGGYLNAYQLAAPFESFE
KLGKQTGILYYVRSDYTSKIDPATGFVDFLKPKYESMAKSKVFFE
SFERIQWNQAKGYFEFEFDYKKMCPSRKFGDYRTRWVVCTFGDTR
YQNRRNKSSGQWETETIDVTAQLKALFAAYGITYNQEDNIKDAIA
AVKYTKFYKQLYWLLRLTLSLRHSVTGTDEDFILSPVADENGVFF
DSRKATDKQPKDADANGAYHIALKGLWNLQQIRQHDWNVEKPKKL
NLAMKNEEWFGFAQKKKFRA
Exemplary ErCas12a wild-type amino acid sequence
SEQ ID NO: 15
MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQTEYRKAIHKKFANDDRFKNMFSAKLISDILPEF
VIHNNNYSASEKEEKTQVIKLFSRFATSFKDYFKNRANCFSADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGFLD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDFDI
TFCHDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYFLHMPITINFKANKTG
FINDRILQYIAKEKDLHVIGIDRGERNLIYVSVIDTCGNIVEQKS
FNIVNGYDYQIKLKQQEGARQIARKEWKEIGKIKEIKEGYLSLVI
HEISKMVIKYNAIIAMEDLSYGFKKGRFKVERQVYQKFETMLINK
LNYLVFKDISITENGGLLKGYQLTYIPDKLKNVGHQCGCIFYVPA
AYTSKIDPTTGFVNIFKFKDLTVDAKREFIKKFDSIRYDSEKNLF
CFTFDYNNFITQNTVMSKSSWSVYTYGVRIKRRFVNGRFSNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWFDFIQNK
RYL
Exemplary ErCas12a variant amino acid sequence
SEQ ID NO: 16
MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQTEYRKAIHKKFANDDRFKNMFSAKLISDILPEF
VIHNNNYSASEKEEKTQVIKLFSRFATSFKDYFKNRANCFSADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGFLD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGRPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDFDI
TFCHDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYLLHMPITINFKANKTG
FINDRILQYIAKEKDLHVIGIDRGERNLIYVSVIDTCGNIVEQKS
FNIVNGYDYQIKLKQQEGARQIARKEWKEIGKIKEIKEGYLSLVI
HEISKMVIKYNAIIAMEDLSYGFKKGRFKVERQVYQKFETMLINK
LNYLVFKDISITENGGLLKGYQLTYIPDKLKNVGHQCGCIFYVPA
AYTSKIDPTTGFVNIFKFKDLTVDAKREFIKKFDSIRYDSEKNLF
CFTFDYNNFITQNTVMSKSSWSVYTYGVRIKRRFVNGRFSNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWFDFIQNK
RYL

Cas12a effector proteins can be, in some embodiments, size-optimized or truncated, for instance via one or more deletions that reduce the size of the effector protein while still retaining gRNA association, target and PAM recognition, and cleavage activities. In some embodiments, CRISPR/Cas effector proteins are bound, covalently or non-covalently, to another polypeptide, nucleotide, or other structure, optionally by means of a linker. Exemplary bound effector proteins and linkers are described by Guilinger et al., Nature Biotech. 32:577-582 (2014), the contents of which is hereby incorporated by reference herein in its entirety.

Additional suitable Cas12a effector proteins and variants thereof will be apparent to the skilled artisan based on the present. Moreover, a number of amino acid sequences of wild-type Cas12a effector protein orthologues are provided in US Publication No. 2021/0079366 A1, the disclosure of which is hereby incorporated herein by reference in its entirety. Exemplary suitable Cas12a effector proteins may include, but are not limited to, those provided in Table 2.

TABLE 2
Exemplary Suitable Variant CRISPR/Cas12a effector proteins

| Cas12a Effector | Additional | |
|---|---|---|
| Protein | Mutation(s) | Effect of mutation(s) |
| Exemplary AsCas12a | K1000G, S1001G | Generates a nickase |
| Variant | | |
| (SEQ ID NO: 6) | | |
| Wild-type AsCas12a | R1226A | Generates a nickase |
| (SEQ ID NO: 1) | | |
| Wild-type AsCas12a | E174R, S542R, | Generates a nickase |
| (SEQ ID NO: 1) | K548R, R1226A | |
| Exemplary AsCas12a | E174R | Increased activity compared |
| Variant | | to wild-type AsCas12a or |
| (SEQ ID NO: 6) | | SEQ ID NO: 6 |
| Exemplary AsCas12a | S186K, R301K, | Increased activity compared |
| Variant | T315R, Q1014R | to wild-type AsCas12a or |
| (SEQ ID NO: 6) | | SEQ ID NO: 6 |
| Exemplary AsCas12a | I1088Y | Increased activity compared |
| Variant | | to wild-type AsCas12a or |
| (SEQ ID NO: 6) | | SEQ ID NO: 6 |
| Exemplary FnCas12a | N602R, F879L | Increased activity compared |
| (SEQ ID NO: 2) | | to wild-type FnCas12a |
| Exemplary FnCas12a | K1013G, R1014G | Generates a nickase |
| (SEQ ID NO: 2) | | |
| Exemplary FnCas12a | R1218A | Generates a nickase |
| (SEQ ID NO: 2) | | |
| Exemplary FnCas12a | E184R, N607R, | Generates a nickase |
| (SEQ ID NO: 2) | K613R, R1218A | |
| Exemplary FnCas12a | E184R | Increased activity compared |
| (SEQ ID NO: 2) | | to wild-type FnCas12a |
| Exemplary FnCas12a | P196K, S334R, | Increased activity compared |
| (SEQ ID NO: 2) | K1026R | to wild-type FnCas12a |
| Exemplary Lb2Cas12a | T778L | Increased activity compared |
| (SEQ ID NO: 3) | | to wild-type Lb2Cas12a |
| Exemplary Lb2Cas12a | K913G, R914G | Generates a nickase |
| (SEQ ID NO: 3) | | |
| Exemplary Lb2Cas12a | R1124A | Generates a nickase |
| (SEQ ID NO: 3) | | |
| Exemplary Lb2Cas12a | K155R, N512R, | Generates a nickase |
| (SEQ ID NO: 3) | K518R, R1124A | |
| Exemplary Lb2Cas12a | K155R | Increased activity compared |
| (SEQ ID NO: 3) | | to wild-type Lb2Cas12a |
| Exemplary Lb2Cas12a | S167K, E271R, | Increased activity compared |
| (SEQ ID NO: 3) | K926R | to wild-type Lb2Cas12a |
| Exemplary LbCas12a | N527R, E795L | Increased activity compared |
| (SEQ ID NO: 4) | | to wild-type LbCas12a |
| Exemplary LbCas12a | K932G, N933G | Generates a nickase |
| (SEQ ID NO: 4) | | |
| Exemplary LbCas12a | R1138A | Generates a nickase |
| (SEQ ID NO: 4) | | |
| Exemplary LbCas12a | D156R, G532R, | Generates a nickase |
| (SEQ ID NO: 4) | K538R, R1138A | |
| Exemplary LbCas12a | D156R | Increased activity compared |
| (SEQ ID NO: 4) | | to wild-type LbCas12a |
| Exemplary LbCas12a | S168K, S286R, | Increased activity compared |
| (SEQ ID NO: 4) | K945R | to wild-type LbCas12a |
| Exemplary MbCas12a | N568R, M825L | Increased activity compared |
| (SEQ ID NO: 5) | | to wild-type MbCas12a |
| Exemplary MbCas12a | K965G, R966G | Generates a nickase |
| (SEQ ID NO: 5) | | |
| Exemplary MbCas12a | R1171A | Generates a nickase |
| (SEQ ID NO: 5) | | |
| Exemplary MbCas12a | D172R, N563R, | Generates a nickase |
| (SEQ ID NO: 5) | K569R, R1171A | |
| Exemplary MbCas12a | D172R | Increased activity compared |
| (SEQ ID NO: 5) | | to wild-type MbCas12a |
| Exemplary MbCas12a | H184K, G292R, | Increased activity compared |
| (SEQ ID NO: 5) | N978R | to wild-type MbCas12a |
| Exemplary MG29-1 | A572R, F849L | Increased activity compared |
| (SEQ ID NO: 14) | | to MG29-1 |
| Exemplary MG29-1 | K983G, R984G | Generates a nickase |
| (SEQ ID NO: 14) | | |
| Exemplary MG29-1 | R1192A | Generates a nickase |
| (SEQ ID NO: 14) | | |
| Exemplary MG29-1 | E172R, N577R, | Generates a nickase |
| (SEQ ID NO: 14) | K583R, R1192A | |
| Exemplary MG29-1 | E172R | Increased activity compared |
| (SEQ ID NO: 14) | | to MG29-1 |
| Exemplary MG29-1 | S184K, R292K, | Increased activity compared |
| (SEQ ID NO: 14) | T306R, K996R | to MG29-1 |
| Exemplary ErCas12a | K969G, K970G | Generates a nickase |
| Variant | | |
| (SEQ ID NO: 16) | | |
| Wild-Type ErCas12a | R1173A | Generates a nickase |
| (SEQ ID NO: 15) | | |
| Wild-Type ErCas12a | K169R, D529R, | Generates a nickase |
| (SEQ ID NO: 15) | K535R, R1173A | |
| Exemplary ErCas12a | S181K, T292R, | Increased activity compared |
| Variant | K982R | to wild-type ErCas12a or |
| (SEQ ID NO: 16) | | SEQ ID NO: 16 |
| Exemplary ErCas12a | K169R | Increased activity compared |
| Variant | | to wild-type ErCas12a or |
| (SEQ ID NO: 16) | | SEQ ID NO: 16 |
| Exemplary ErCas12a | D1055Y | Increased activity compared |
| Variant | | to wild-type ErCas12a or |
| (SEQ ID NO: 16) | | SEQ ID NO: 16 |

### Fusion Proteins for Base Editing

In one aspect, the present disclosure provides Cas12a effector proteins fused to one or more heterologous protein domains (“fusion proteins”) for base editing as described herein. In some embodiments, one or more heterologous protein domains comprise or are deaminase domains and/or polypeptides. Any deaminase domain and/or polypeptide useful for base editing may be used in a fusion protein of the present disclosure. A cytosine base editor (CBE), as used herein, comprises a cytosine deaminase. An adenine base editor (ABE), as used herein, comprises an adenine deaminase.

### Cytosine Deaminase

In some embodiments, a deaminase comprises or is a cytosine deaminase or a cytidine deaminase. A “cytosine deaminase” and “cytidine deaminase” as used herein refer to a polypeptide or domain thereof that catalyzes or is capable of catalyzing cytosine deamination in that the polypeptide or domain catalyzes or is capable of catalyzing the removal of an amine group from a cytosine base. Thus, a cytosine deaminase may result in conversion of cytosine to a thymidine (through a uracil intermediate), causing a C to T conversion, or a G to A conversion in the complementary strand in the genome. Thus, in some embodiments, a cytosine deaminase encoded by a polynucleotide of the present disclosure generates a C to T conversion in the sense (e.g., “+”: template) strand of the target nucleic acid and/or a G to A conversion in antisense (e.g., complementary) strand of the target nucleic acid. In some embodiments, a cytosine deaminase encoded by a polynucleotide of the present disclosure generates a C to T, G, or A conversion in the complementary strand in the genome.

In some embodiments, a cytosine deaminase may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain may be a cytidine deaminase domain, catalyzing the hydrolytic deamination of cytosine to uracil. In some embodiments, a cytosine deaminase may be a variant of a naturally-occurring cytosine deaminase, including, but not limited to, a primate (e.g., a human, monkey, chimpanzee, gorilla), a dog, a cow, a rat, or a mouse cytosine deaminase. Thus, in some embodiments, an cytosine deaminase useful with the invention may be about 70% to about 100% identical to a wild-type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, a cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2gl9570), and evolved versions of the same. Evolved deaminases are disclosed in, for example, U.S. Pat. No. 10,113,163, Gaudelli et al., Nature 551 (7681): 464-471 (2017)) and Thuronyi et al., Nature Biotechnology 37:1070-1079 (2019), each of which are incorporated by reference herein for their disclosure of deaminases and evolved deaminases. In some embodiments, a cytosine deaminase may be an APOBEC1 deaminase having the amino acid sequence of SEQ ID NO: 57. In some embodiments, a cytosine deaminase may be an APOBEC3A deaminase having the amino acid sequence of SEQ ID NO: 58. In some embodiments, a cytosine deaminase may be a CDA1 deaminase, optionally a CDA1 having the amino acid sequence of SEQ ID NO: 59. In some embodiments, a cytosine deaminase may be a FERNY deaminase, optionally a FERNY having the amino acid sequence of SEQ ID NO: 60. In some embodiments, a cytosine deaminase may be an rAPOBEC1 deaminase, optionally an rAPOBEC1 deaminase having the amino acid sequence of SEQ ID NO: 61. In some embodiments, a cytosine deaminase may be an hAID deaminase, optionally an hAID having the amino acid sequence of SEQ ID NO: 62 or SEQ ID NO: 63. In some embodiments, a cytosine deaminase may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., “evolved deaminases”) (see, e.g., SEQ ID NO: 64, SEQ ID NO: 65, SEQ ID NO: 66). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of any one of SEQ ID NOs: 57-66 (e.g., at least 80%, at least 85%, at least 90%, at least 92%, at least 95%, at least 96%, at least 97%, at least 98%, at least 99%, or at least 99.5% identical to the amino acid sequence of any one of SEQ ID NOs: 57-66). In some embodiments, a polynucleotide encoding a cytosine deaminase may be codon optimized for expression in a mammal and the codon optimized polynucleotide may be about 70% to 99.5% identical to the reference polynucleotide.

In some embodiments a cytosine base editor (CBE) of the present disclosure comprises a cytidine deaminase fused to a Cas12a nickase tethered to one (BE3) or two (BE4) monomers of uracil glycosylase inhibitor (UGI). In some embodiments the cytidine deaminase is PpAPOBEC1, e.g., having the following sequence:

(SEQ ID NO: 56)
MTSEKGPSTGDPTLRRRIESWEFDVFYDPRELRKETCLLYEIKWG
MSRKIWRSSGKNTTNHVEVNFIKKFTSERRFHSSISCSITWFLSW
SPCWECSQAIREFLSQHPGVTLVIYVARLFWHMDQRNRQGLRDLV
NSGVTIQIMRASEYYHCWRNFVNYPPGDEAHWPQYPPLWMMLYAL
ELHCIILSLPPCLKISRRWQNHLAFFRLHLQNCHYQTIPPHILLA
TGLIHPSVTWR.

In some embodiments the cytosine base editor comprises PpAPOBEC1 fused to a Cas12a nickase tethered to two (BE4) monomers of uracil glycosylase inhibitor (UGI), e.g., as described in Yu et al., Nat. Comm. 11:2052, 2020 and WO2020160517A1 (the entire contents of each of which are incorporated herein by reference).

### Exemplary Cytosine Deaminase Sequences

(SEQ ID NO: 57)
SSETGPVAVDPTLRRRIEPHEFEVFFDPRELRKETCLLYEINWGG
RHSIWRHTSQNTNKHVEVNFIEKFTTERYFCPNTRCSITWFLSWS
PCGECSRAITEFLSRYPHVTLFIYIARLYHHADPRNRQGLRDLIS
SGVTIQIMTEQESGYCWRNFVNYSPSNEAHWPRYPHLWVRLYVLE
LYCIILGLPPCLNILRRKQPQLTFFTIALQSCHYQRLPPHILWAT
GLK
(SEQ ID NO: 58)
MEASPASGPRHLMDPHIFTSNFNNGIGRHKTYLCYEVERLDNGTS
VKMDQHRGFLHNQAKNLLCGFYGRHAELRFLDLVPSLQLDPAQIY
RVTWFISWSPCFSWGCAGEVRAFLQENTHVRLRIFAARIYDYDPL
YKEALQMLRDAGAQVSIMTYDEFKHCWDTFVDHQGCPFQPWDGLD
EHSQALSGRLRAILQNQGN
(SEQ ID NO: 59)
MTDAEYVRIHEKLDIYTFKKQFFNNKKSVSHRCYVLFELKRRGER
RACFWGYAVNKPQSGTERGIHAEIFSIRKVEEYLRDNPGQFTINW
YSSWSPCADCAEKILEWYNQELRGNGHTLKIWACKLYYEKNARNQ
IGLWNLRDNGVGLNVMVSEHYQCCRKIFIQSSHNQLNENRWLEKT
LKRAEKRRSELSIMIQVKILHTTKSPAV
(SEQ ID NO: 60)
FERNYDPRELRKETYLLYEIKWGKSGKLWRHWCQNNRTQHAEVYF
LENIFNARRENPSTHCSITWYLSWSPCAECSQKIVDFLKEHPNVL
EIYVARLYYHEDERNRQGLRDLVNSGVTIRIMDLPDYNYCWKTFV
SDQGGDEDYWPGHFAPWIKQYSLKL
(SEQ ID NO: 61)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
(SEQ ID NO: 62)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGL
(SEQ ID NO: 63)
MDSLLMNRREFLYQFKNVRWAKGRRETYLCYVVKRRDSATSFSLD
FGYLRNKNGCHVELLFLRYISDWDLDPGRCYRVTWFISWSPCYDC
ARHVADFLRGNPNLSLRIFTARLYFCEDRKAEPEGLRRLHRAGVQ
IAIMTFKDYFYCWNTFVENHGRTFKAWEGLHENSVRLSRQLRRIL
LPLYEVDDLRDAFRTCT
(SEQ ID NO: 64)
TDAEYVRIHEKLDIYTFKKQFSNNKKSVSHRCYVLFELKRRGERR
ACFWGYAVNKPQSGTERGIHAEIFSIRKVEEYLRDNPGQFTINWY
SSWSPCADCAEKILEWYNQELRGNGHTLKIWVCKLYYEKNARNQI
GLWNLRDNGVGLNVMVSEHYQCCRKIFIQSSHNQLNENRWLEKTL
KRAEKRRSELSIMFQVKILHTTKSPAV
(SEQ ID NO: 65)
SSKTGPVAVDPTLRRRIEPHEFEVFFDPRELRKETCLLYEINWGG
RHSIWRHTSQNTNKHVEVNFIEKFTTERYFCPNTRCSITWFLSWS
PCGECSRAITEFLSRYPNVTLFIYIARLYHLANPRNRQGLRDLIS
SGVTIQIMTEQESGYCWHNFVNYSPSNESHWPRYPHLWVRLYVLE
LYCIILGLPPCLNILRRKQSQLTSFTIALQSCHYQRLPPHILWAT
GLK
(SEQ ID NO: 66)
SFERNYDPRELRKETYLLYEIKWGKSGKLWRHWCQNNRTQHAEVY
FLENIFNARRFNPSTHCSITWYLSWSPCAECSQKIVDFLKEHPNV
NLEIYVARLYYPENERNRQGLRDLVNSGVTIRIMDLPDYNYCWKT
FVSDQGGDEDYWPGHFAPWIKQYSLKL

### Adenine Deaminase

In some embodiments, a deaminase comprises or is an adenine deaminase or an adenosine deaminase. An “adenine deaminase” and “adenosine deaminase” as used herein refer to a polypeptide or domain thereof that catalyzes or is capable of catalyzing the hydrolytic deamination (e.g., removal of an amine group from adenine) of adenine or adenosine. In some embodiments, an adenine deaminase may catalyze the hydrolytic deamination of adenosine or deoxy adenosine to inosine or deoxyinosine, respectively. In some embodiments, an adenine deaminase may catalyze the hydrolytic deamination of adenine or adenosine in DNA. In some embodiments, an adenine deaminase encoded by a nucleic acid may generate an A to G conversion in the sense (e.g., “+”: template) strand of the target nucleic acid or a T to C conversion in the antisense (e.g., complementary) strand of the target nucleic acid.

An adenine deaminase may be any known or later identified adenine deaminase from any organism (see, e.g., U.S. Pat. No. 10,113,163, which is incorporated by reference herein for its disclosure of adenine deaminases).

In some embodiments, an adenine deaminase may be a variant of a naturally-occurring adenine deaminase. Thus, in some embodiments, an adenine deaminase may be about 70% to 100% identical to a wild-type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, an adenine deaminase does not occur in nature and may be referred to as an engineered, mutated or evolved adenine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus). In some embodiments, a polynucleotide encoding an adenine deaminase polypeptide/domain may be codon optimized for expression in a mammal and the codon optimized polynucleotide may be about 70% to 99.5% identical to the reference polynucleotide.

In some embodiments, an adenine deaminase domain may be a wild-type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, a TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild-type E. coli TadA comprises the amino acid sequence of SEQ ID NO: 71. In some embodiments, a mutated/evolved E. coli TadA* comprises the amino acid sequence of SEQ ID NOs: 72-75 (e.g., SEQ ID NOs: 72, 73, 74, or 75). In some embodiments, a polynucleotide encoding a TadA/TadA* may be codon optimized for expression in a mammal. In some embodiments, an adenine deaminase may comprise all or a portion of an amino acid sequence of any one of SEQ ID NOs: 76-81. In some embodiments, an adenine deaminase may comprise all or a portion of an amino acid sequence of any one of SEQ ID NOs: 71-81.

In some embodiment an adenine base editor of the present disclosure comprises an adenosine deaminase with one or more mutations to reduce undesirable RNA editing activity. In some embodiments, the base editor comprises an engineered E. coli TadA, e.g., with the mutations found in ABEs 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, ABEmax as described in Gaudelli et al., Nature 551 (7681): 464-471, 2017 and Koblan et al., Nat. Biotechnol. 36 (9): 843-846, 2018 (the entire contents of each of which are incorporated herein by reference) or any of the ABE8s variants, e.g., ABE8.17-m, described in Gaudelli et al., Nat. Biotechnol. 38:892-900, 2020 and US20210130805A1 (the entire contents of each of which are incorporated herein by reference). The mutations can include substitution with any other amino acid other than the wild-type amino acid. In some embodiments the substitution is with alanine or glycine. For example, the engineered E. coli TadA sequence present in ABE7.10 (TadA*7.10) is as follows:

(SEQ ID NO: 67)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGVRNAKTGAAGSLMDVLHYPGMNHRVEITEGI
LADECAALLCYFFRMPRQVFNAQKKAQSSTD.

In ABE7.10, a wild-type E. coli TadA sequence is fused to this engineered E. coli TadA sequence using a 32 amino acid linker, forming a heterodimer, the sequence of which is as follows:

(SEQ ID NO: 68)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTD.

As a further example, ABE8.17-m comprises a monomeric construct containing TadA*7.10 with V82S and Q154R mutations (TadA*8.17) as follows:

(SEQ ID NO: 69)
MSEVEFSHEYWMRHALTLAKRARDEREVPVGAVLVLNNRVIGEGW
NRAIGLHDPTAHAEIMALRQGGLVMQNYRLIDATLYSTFEPCVMC
AGAMIHSRIGRVVFGVRNAKTGAAGSLMDVLHYPGMNHRVEITEG
ILADECAALLCYFFRMPRRVFNAQKKAQSSTD.

In some embodiments an adenine base editor (ABE) of the present disclosure comprises an adenosine deaminase fused to a Cas12a nickase, e.g., a Cas12a nickase fused to a wild-type E. coli TadA, e.g., of SEQ ID NO: 68:

(SEQ ID NO: 70)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD.

### Exemplary Adenine Deaminase Sequences

(SEQ ID NO: 71)
SEVEFSHEYWMRHALTLAKRAWDEREVPVGAVLVHNNRVIGEGWN
RPIGRHDPTAHAEIMALRQGGLVMQNYRLIDATLYVTLEPCVMCA
GAMIHSRIGRVVFGARDAKTGAAGSLMDVLHHPGMNHRVEITEGI
LADECAALLSDFFRMRRQEIKAQKKAQSSTD
(SEQ ID NO: 72)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGVRNAKTGAAGSLMDVLHYPGMNHRVEITEGI
LADECAALLCYFFRMPRQVFNAQKKAQSSTD
(SEQ ID NO: 73)
SEVEFSHEYWMRHA
LTLAKRAWDEREVPVGAVLVLNNRVIGEGWNRSIGLHDPTAHAEI
MALRQGGLVMQNYRLIDATLYVTFEPCVMCAGAMIHSRIGRVVFG
VRNAKTGAAGSLMDVLHYPGMNHRVEITEGILADECAALLCYFFR
MRRQVFNAQKKAQSSTD
(SEQ ID NO: 74)
SEVEFSHEYWMRHALTLAKRALDEREVPVGAVLVLNNRVIGEGWN
RAIGLHDPTAHAEIMALRQGGLVMQNYRLIDATLYVTFEPCVMCA
GAMIHSRIGRVVFGVRNAKTGAAGSLMDVLHYPGMNHRVEITEGI
LADECNALLCYFFRMRRQVFNAQKKAQSSTD
(SEQ ID NO: 75)
SEVEFSHEYWMRHALTLAKRALDEREVPVGAVLVLNNRVIGEGWN
RAIGLHDPTAHAEIMALRQGGLVMQNYRLIDATLYVTFEPCVMCA
GAMIHSRIGRVVFGVRNAKTGAAGSLMDVLHYPGMNHRVEITEGI
LADECNALLCYFFRMPRQVFNAQKKAQSSTD
(SEQ ID NO: 76)
SEVEFSHEYWMRHALTLAKRAWDEREVPVGAVLVHNNRVIGEGWN
RPIGRHDPTAHAEIMALRQGGLVMQNYRLIDATLYVTLEPCVMCA
GAMIHSRIGRVVFGARDAKTGAAGSLMDVLHHPGMNHRVEITEGI
LADECAALLSDFFRMRRQEIKAQKKAQSSTD
(SEQ ID NO: 77)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGVRNSKRGAAGSLMNVLNYPGMNHRVEITEGI
LADECAALLCDFYRMPRQVFNAQKKAQSSIN
(SEQ ID NO: 78)
SEVEFSHEYWMRHALTLAKRARDEREVPVGAVLVLNNRVIGEGWN
RAIGLHDPTAHAEIMALRQGGLVMQNYRLYDATLYSTFEPCVMCA
GAMIHSRIGRVVFGVRNAKTGAAGSLMDVLHHPGMNHRVEITEGI
LADECAALLCRFFRMPRRVFNAQKKAQSSTD
(SEQ ID NO: 79)
SEVEFSHEYWMRHALTLAKRAWDEREVPVGAVLVHNNRVIGEGWN
RPIGRHDPTAHAEIMALRQGGLVMQNYRLIDATLYVTLEPCVMCA
GAMIHSRIGRVVFGARDAKTGAAGSLMDVLHHPGMNHRVEITEGI
LADECAALLSDFFRMRRQEIKAQKKAQSSTDSGGSSGGSSGSETP
GTSESATPESSGGSSGGSSEVEFSHEYWMRHALTLAKRARDEREV
PVGAVLVLNNRVIGEGWNRAIGLHDPTAHAEIMALRQGGLVMQNY
RLIDATLYVTFEPCVMCAGAMIHSRIGRVVFGVRNAKTGAAGSLM
DVLHYPGMNHRVEITEGILADECAALLCYFFRMPRQVFNAQKKAQ
SSTD
(SEQ ID NO: 80)
MSEVEFSHEYWMRHALTLAKRARDEREVPVGAVLVLNNRVIGEGW
NRAIGLHDPTAHAEIMALRQGGLVMQNYRLYDATLYSTFEPCVMC
AGAMIHSRIGRVVFGVRNAKTGAAGSLMDVLHHPGMNHRVEITEG
ILADECAALLCRFFRMPRRVFNAQKKAQSSTD
(SEQ ID NO: 81)
MSEVEFSHEYWMRHALTLAKRARDEREVPVGAVLVLNNRVIGEGW
NRAIGLHDPTAHAEIMALRQGGLVMQNYRLIDATLYVTFEPCVMC
AGAMIHSRIGRVVFGVRNSKRGAAGSLMNVLNYPGMNHRVEITEG
ILADECAALLCDFYRMPRQVFNAQKKAQSSIN

In some embodiments, a nucleic acid of the present disclosure may further encode a glycosylase inhibitor (e.g., a uracil glycosylase inhibitor (UGI) such as uracil-DNA glycosylase inhibitor). Thus, in some embodiments, a nucleic acid encoding a Cas12a effector protein and a cytosine deaminase and/or adenine deaminase may further encode a glycosylase inhibitor, optionally wherein the glycosylase inhibitor may be codon optimized for expression in a mammal. In some embodiments, present disclosure provides fusion proteins comprising a Cas12a effector protein and a UGI and/or one or more polynucleotides encoding the same, optionally wherein the one or more polynucleotides may be codon optimized for expression in a mammal. In some embodiments, the present disclosure provides fusion proteins comprising a Cas12a effector protein, a deaminase domain (e.g., an adenine deaminase domain and/or a cytosine deaminase domain) and a UGI and/or one or more polynucleotides encoding the same, optionally wherein the one or more polynucleotides may be codon optimized for expression in a mammal. In some embodiments, the invention provides fusion proteins, wherein a Cas12a effector protein, a deaminase domain, and/or a UGI may be fused to any combination of peptide tags and affinity polypeptides as described herein, which may thereby recruit the deaminase domain and/or UGI to the Cas12a effector protein and to a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” or “UGI” may be any protein or polypeptide or domain thereof that is capable of inhibiting a uracil-DNA glycosylase base-excision repair enzyme. In some embodiments, a UGI comprises a wild-type UGI or a fragment thereof. In some embodiments, a UGI is about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI. In some embodiments, a UGI may comprise the amino acid sequence of:

(SEQ ID NO: 82)
TNLSDIIEKETGKQLVIQESILMLPEEVEEVIGNKPESDILVHTA
YDESTDENVMLLTSDAPEYKPWALVIQDSNGENKIKML

or a polypeptide having about 70% to about 99.5% identity to the amino acid sequence of SEQ ID NO: 82 (e.g., at least 80%, at least 85%, at least 90%, at least 92%, at least 95%, at least 96%, at least 97%, at least 98%, at least 99%, or at least 99.5% identical to the amino acid sequence of SEQ ID NO: 82). For example, in some embodiments, a UGI may comprise a fragment of the amino acid sequence of SEQ ID NO: 82 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO: 82. In some embodiments, a UGI may be a variant of a known UGI (e.g., SEQ ID NO: 82) having about 70% to about 99.5% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGI may be codon optimized for expression in a mammal and the codon optimized polynucleotide may be about 70% to about 99.5% identical to the reference polynucleotide.
Guide RNA (gRNA) Molecules

A gRNA molecule or gRNA for use in a CRISPR/Cas12a genome editing system generally includes a targeting domain and a complementarity domain (alternately referred to as a “handle”). It should also be noted that, in gRNAs for use with Cas12a, the targeting domain is usually present at or near the 3′ end, rather than the 5′ end as in connection with Cas9 gRNAs (the handle is at or near the 5′ end of a Cas12a gRNA).

Those of skill in the art will appreciate, however, that although structural differences may exist between gRNAs from different prokaryotic species, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple CRISPR/Cas effector proteins. For this reason, unless otherwise specified, the term gRNA should be understood to encompass any suitable gRNA that can be used with any CRISPR/Cas effector system, and not only those gRNAs that are compatible with a particular species of Cas12a. By way of illustration, the term gRNA can, in some embodiments, include a gRNA for use with any CRISPR/Cas effector protein occurring in a Class 2 CRISPR system, such as a Type V CRISPR system, or a CRISPR/Cas effector protein derived or adapted therefrom.

In some embodiments a method or system of the present disclosure may use more than one gRNA. In some embodiments, two or more gRNAs may be used to create two or more double strand breaks in the genome of a cell.

In some embodiments using more than one gRNA, a double-strand break may be caused by a dual-gRNA paired “nickase” strategy.

gRNA Design

Methods for selection and validation of target nucleic sequences as well as off-target analyses have been described previously, e.g., in Fu et al., Nat Biotechnol 32(3): 279-84 (2014), Heigwer et al., Nat methods 11(2): 122-3 (2014); Bae et al., Bioinformatics 30(10): 1473-5 (2014); and Xiao et al. Bioinformatics 30(8): 1180-1182 (2014). As a non-limiting example, gRNA design may involve the use of a software tool to optimize the choice of potential target nucleic sequences corresponding to a user's target nucleic sequence, e.g., to minimize total off-target activity across the genome. While off-target activity is not limited to cleavage, the cleavage efficiency at each off-target nucleic sequence can be predicted, e.g., using an experimentally-derived weighting scheme. These and other guide selection methods are described in detail in Park et al., Bioinformatics 34 (6): 1077-1079 (2018), the disclosure of which is hereby incorporated herein by reference in its entirety.

gRNA Modifications

In some embodiments, gRNAs as used herein may be modified or unmodified gRNAs. In some embodiments, gRNAs as used herein may be modified for increased activity compared to unmodified gRNAs. In some embodiments, a gRNA may include one or more modifications. In some embodiments, the one or more modifications may include a phosphorothioate linkage modification, a phosphorodithioate (PS2) linkage modification, a 2′-O-methyl modification, or combinations thereof. In some embodiments, the one or more modifications may be at the 5′ end of the gRNA, at the 3′ end of the gRNA, or combinations thereof.

In some embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA. In some embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in some embodiments, the DNA extension may be 1, 2, 3, 4, 5, 10, 15, 20, or 25 DNA bases long. In some embodiments, the DNA extension may include one or more DNA bases selected from adenine (A), guanine (G), cytosine (C), or thymine (T). In some embodiments, the DNA extension includes the same DNA bases. For example, the DNA extension may include a stretch of adenine (A) bases. In some embodiments, the DNA extension may include a stretch of thymine (T) bases. In some embodiments, the DNA extension includes a combination of different DNA bases.

Exemplary suitable 5′ extensions for Cas12a guide RNAs are provided in Table 3 below:

TABLE 3
Exemplary Cas12a gRNA 5′ Extensions

| SEQ ID NO: | 5′ extension sequence | 5′ modification |
|---|---|---|
| N/A | rCrUrUrUrU | +5 RNA |
| 17 | rArArGrArCrCrUrUrUrU | +10 RNA |
| 18 | rArUrGrUrGrUrUrUrUrUrGrUrCrArArArArGrArCr | +25 RNA |
| | CrUrUrUrU | |
| 19 | rArGrGrCrCrArGrCrUrUrGrCrCrGrGrUrUrUrUrUr | +60 RNA |
| | UrArGrUrCrGrUrGrCrUrGrCrUrUrCrArUrGrUrGrU | |
| | rUrUrUrUrGrUrCrArArArArGrArCrCrUrUrUrU | |
| N/A | CTTTT | +5 DNA |
| 20 | AAGACCTTTT | +10 DNA |
| 21 | ATGTGTTTTTGTCAAAAGACCTTTT | +25 DNA |
| 22 | AGGCCAGCTTGCCGGTTTTTTAGTCGTGCTGC | +60 DNA |
| | TTCATGTGTTTTTGTCAAAAGACCTTTT | |
| 23 | TTTTTGTCAAAAGACCTTTT | +20 DNA |
| 24 | GCTTCATGTGTTTTTGTCAAAAGACCTTTT | +30 DNA |
| 25 | GCCGGTTTTTTAGTCGTGCTGCTTCATGTGTTT | +50 DNA |
| | TTGTCAAAAGACCTTTT | |
| 26 | TAGTCGTGCTGCTTCATGTGTTTTTGTCAAAA | +40 DNA |
| | GACCTTTT | |
| 27 | C*C*GAAGTTTTCTTCGGTTTT | +20 DNA + 2xPS |
| 28 | T*T*TTTCCGAAGTTTTCTTCGGTTTT | +25 DNA + 2xPS |
| 29 | A*A*CGCTTTTTCCGAAGTTTTCTTCGGTTTT | +30 DNA + 2xPS |
| 30 | G*C*GTTGTTTTCAACGCTTTTTCCGAAGTTTT | +41 DNA + 2xPS |
| | CTTCGGTTTT | |
| 31 | G*G*CTTCTTTTGAAGCCTTTTTGCGTTGTTTT | +62 DNA + 2xPS |
| | CAACGCTTTTTCCGAAGTTTTCTTCGGTTTT | |
| 32 | A*T*GTGTTTTTGTCAAAAGACCTTTT | +25 DNA + 2xPS |
| 33 | AAAAAAAAAAAAAAAAAAAAAAAAA | +25 A |
| 34 | TTTTTTTTTTTTTTTTTTTTTTTTT | +25 T |
| 35 | mA*mU*rGrUrGrUrUrUrUrUrGrUrCrArArArArGr | +25 RNA + 2xPS |
| | ArCrCrUrUrUrU | |
| 36 | mA*mA*rArArArArArArArArArArArArArArArAr | Poly A RNA + |
| | ArArArArArArA | 2xPS |
| 37 | mU*mU*rUrUrUrUrUrUrUrUrUrUrUrUrUrUrUrUr | Poly U RNA + |
| | UrUrUrUrUrUrU | 2xPS |

In some embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In some embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension”. In some embodiments, a gRNA used herein includes an RNA extension at the 5′ end of the gRNA, the 3′ end of the gRNA, or a combination thereof. In some embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in some embodiments, the RNA extension may be 1, 2, 3, 4, 5, 10, 15, 20, or 25 RNA bases long. Exemplary suitable 5′ extensions for Cas12a guide RNAs are provided in Table 3 above. In some embodiments, the RNA extension may include one or more RNA bases selected from adenine (rA), guanine (rG), cytosine (rC), or uracil (rU), in which the “r” represents RNA. 2′-hydroxy. In some embodiments, the RNA extension includes the same RNA bases. For example, the RNA extension may include a stretch of adenine (rA) bases. In some embodiments, the RNA extension includes a combination of different RNA bases. In some embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In some embodiments, the one or more modifications may be at the 5′ end of the gRNA, at the 3′ end of the gRNA, or combinations thereof. In some embodiments, a gRNA including a RNA extension may comprise a sequence set forth herein.

It is contemplated that gRNAs used herein may also include an RNA extension and a DNA extension. In some embodiments, the RNA extension and DNA extension may both be at the 5′ end of the gRNA, the 3′ end of the gRNA, or a combination thereof. In some embodiments, the RNA extension is at the 5′ end of the gRNA and the DNA extension is at the 3′ end of the gRNA. In some embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a CRISPR/Cas effector protein, e.g., an Cas12a effector protein, to form an RNP, which is then employed to edit a target cell, e.g., a pluripotent stem cell or a progeny thereof.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as a stem loop structure of a Cas12a gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′) G anti-reverse cap analog (ARCA)), as shown below:

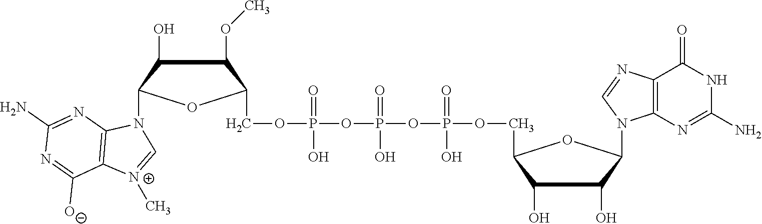

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a poly A tract. The poly A tract can be added to a gRNA during chemical or enzymatic synthesis, using a polyadenosine polymerase (e.g., E. coli Poly(A) Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

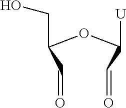

wherein “U” can be an unmodified or modified uridine.

The 3′ terminal U ribose can be modified with a 2′3′ cyclic phosphate as shown below:

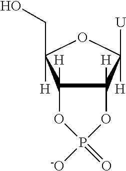

wherein “U” can be an unmodified or modified uridine.

Guide RNAs can contain 3′ nucleotides that can be stabilized against degradation, e.g., by incorporating one or more of the modified nucleotides described herein. In some embodiments, uridines can be replaced with modified uridines, e.g., 5-(2-amino) propyl uridine, and 5-bromo uridine, or with any of the modified uridines described herein: adenosines and guanosines can be replaced with modified adenosines and guanosines, e.g., with modifications at the 8-position, e.g., 8-bromo guanosine, or with any of the modified adenosines or guanosines described herein.

In some embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate (PhTx) group. In some embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4 carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In some embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In some embodiments, a gRNA comprises a 4′-S, 4′-Se or a 4-C-aminomethyl-2′-O-Me modification.

In some embodiments, deaza nucleotides, e.g., 7-deaza-adenosine, can be incorporated into a gRNA. In some embodiments, O- and N-alkylated nucleotides, e.g., N6-methyl adenosine, can be incorporated into a gRNA. In some embodiments, one or more or all of the nucleotides in a gRNA are deoxynucleotides.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g., bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxy methyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Additional suitable gRNA modifications will be apparent to those of ordinary skill in the art based on the present disclosure. Suitable gRNA modifications include, for example, those described in PCT Publication Nos. WO2019070762A1, WO2016089433A1, WO2016164356A1, or WO2017053729A1, the entire contents of each of which are incorporated herein by reference.

Exemplary gRNAs

Non-limiting examples of guide RNAs suitable for certain embodiments embraced by the present disclosure are provided herein. Those of ordinary skill in the art will be able to envision suitable guide RNA sequences for a specific CRISPR effector protein, e.g., a Cas12a effector protein, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and contain uracil instead of thymidine nucleotides. Suitable gRNA scaffold sequences are known to those of ordinary skill in the art. For a Cas12a, for example, a suitable scaffold sequence comprises a sequence selected from Table 4 or a pair of sequences selected from Table 5. In Table 5, it is understood that a “modulator sequence” listed herein may constitute the nucleotide sequence of a modulator nucleic acid. Alternatively, additional nucleotide sequences can be comprised in the modulator nucleic acid 5′ and/or 3′ to a “modulator sequence” listed herein. In the consensus PAM sequences of Table 4 and Table 5, N represents A, C, G or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 4
Exemplary Single gRNA Scaffold Sequences

| Cas | | |
|---|---|---|
| Protein | Scaffold Sequence | PAM |
| AsCas12a | UAAUUUCUACUCUUGUAGA | 5′ TTTN |
| | (SEQ ID NO: 38) | |
| FnCas12a | UAAUUUUCUACUUGUUGUAGA | 5′ TTN |
| | (SEQ ID NO: 39) | |
| LbCas12a | UAAUUUCUACUAAGUGUAGA | 5′ TTTN |
| | (SEQ ID NO: 40) | |
| ErCas12a | UAAUUUCUACUCUUGUAGA | 5′ TTTN or |
| | (SEQ ID NO: 38) | 5′CTTN |
| | AUCUACAACAGUAGA | |
| | (SEQ ID NO: 41) | |
| | AUCUACAAAAGUAGA | |
| | (SEQ ID NO: 42) | |
| | GGAAUUUCUACUCUUGUAGA | |
| | (SEQ ID NO: 43) | |

TABLE 5
Exemplary Dual gRNA Scaffold Sequences

| | | Targeter | |
|---|---|---|---|
| Cas | | Stem | |
| Protein | Modulator Sequence | Sequence | PAM |
| AsCas12a | UAAUUUCUAC | GUAGA | 5′ TTTN |
| | (SEQ ID NO: 44) | | |
| FnCas12a | UAAUUUUCUACU | GUAGA | 5′ TTN |
| | (SEQ ID NO: 45) | | |
| LbCas12a | UAAUUUCUAC | GUAGA | 5′ TTTN |
| | (SEQ ID NO: 46) | | |
| ErCas12a | UAAUUUCUAC | GUAGA | 5′ TTTN |
| | (SEQ ID NO: 44) | | or |
| | AUCUAC | GUAGA | 5′CTTN |
| | GGAAUUUCUAC | GUAGA | |
| | (SEQ ID NO: 48) | | |
| | UAAUUCCCAC | GUGGG | |
| | (SEQ ID NO: 49) | | |

Additional exemplary gRNA sequences include:

(SEQ ID NO: 52)
GTTAAGTTATATAGAATAATTTCTACTGTTGTAGA,
(SEQ ID NO: 53)
CTCTACAACTGATAAAGAATTTCTACTTTTGTAGAT
and
(SEQ ID NO: 54)
GTCTGGCCCCAAATTTTAATTTCTACTGTTGTAGAT

For an MG29-1 effector protein, a suitable guide RNA may comprise a backbone sequence comprising TAATTTCTACTGTTGTAGAT (SEQ ID NO: 55).

It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

### Systems and Methods for Editing the Genome of a Cell

In one aspect the present disclosure provides systems for editing the genome of a cell. In some embodiments, a Cas12a effector protein causes a double-strand break. In some embodiments a Cas12a effector protein causes a single-strand break, e.g., in some embodiments a Cas12a effector protein is a nickase.

Genome editing systems and methods comprising a Cas12a effector protein can be implemented (e.g., administered or delivered to a cell or a subject) in a variety of ways, and different implementations may be suitable for distinct applications. For instance, a genome editing system is implemented. In some embodiments, as a protein/RNA complex (a ribonucleoprotein, or RNP). In some embodiments, a genome editing system and/or method is implemented as one or more nucleic acids encoding a Cas12a effector protein and guide RNA components described herein (optionally with one or more additional components). In some embodiments, a genome editing system and/or method is implemented as one or more vectors comprising such nucleic acids, for instance a viral vector such as an adeno-associated virus. In some embodiments, a genome editing system and/or method is implemented as a combination of any of the foregoing. Additional or modified implementations that operate according to the principles set forth herein will be apparent to the skilled artisan and are within the scope of this disclosure.

In some embodiments, genome editing systems and/or methods may be capable of target disruption, such as target mutation or alteration, such as leading to gene knockout. In some embodiments, genome editing systems and/or methods may involve replacement of particular target sites, such as leading to target correction. In some embodiments, genome editing systems and/or methods may involve removal of particular target sites, such as leading to target deletion. In some embodiments, genome editing systems and methods comprise a Cas12a effector protein comprising a Cas12a dual nickase for homology directed repair (HDR). In some embodiments, genome editing systems and/or methods may involve modulation of target site functionality, such as target site activity or accessibility, leading for instance to (transcriptional and/or epigenetic) gene or genomic region activation or gene or genomic region silencing.

The present disclosure further provides a method of altering a cell, e.g., altering the structure, e.g., altering the sequence, of a target nucleic acid of a cell, comprising contacting the cell with: (a) a gRNA molecule as described herein and (b) a Cas12a effector protein or fusion protein as described herein, and optionally, (c) a second gRNA molecule as described herein. In another aspect, disclosed herein is a method of treating a subject (e.g., a subject suffering from a disease, e.g., a cancer), e.g., altering the structure, e.g., sequence, of a target nucleic acid of the subject, comprising contacting the subject (or a cell from the subject) with: (a) a gRNA as described herein; and (b) a Cas12a effector protein or fusion protein as described herein, and optionally, (c) a second gRNA molecule as described herein.

In some embodiments, the contacting comprises delivering to the cell a Cas12a effector protein or fusion protein of (b) as a protein or an mRNA, and a nucleic acid molecule which encodes (a) and optionally (c). In some embodiments, the contacting comprises delivering to the cell a Cas12a effector protein or fusion protein of (b) as a protein or an mRNA, the gRNA of (a) as an RNA, and optionally the second gRNA of (c), as an RNA.

In some embodiments, (a) and (b) are present on one nucleic acid molecule, e.g., one vector, e.g., one viral vector, e.g., an AAV vector. Exemplary AAV vectors that may be used in any of the described compositions and methods include an AAV1 vector, a modified AAV1 vector, an AAV2 vector, a modified AAV2 vector, an AAV3 vector, an AAV4 vector, a modified AAV4 vector, an AAV5 vector, a modified AAV5 vector, a modified AAV3 vector, an AAV6 vector, a modified AAV6 vector, an AAV7 vector, a modified AAV7 vector, an AAV8 vector, an AAV5 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector. In some embodiments, (a) is present on a first nucleic acid molecule, e.g., a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In some embodiments, (a) and (c) are be present on one nucleic acid molecule, e.g., one vector, e.g., one viral vector, e.g., one AAV vector. In some embodiments, (a) and (c) are on different vectors. For example, (a) may be present on a first nucleic acid molecule, e.g., a first vector, e.g., a first viral vector, e.g., a first AAV vector; and (c) may be present on a second nucleic acid molecule, e.g., a second vector, e.g., a second vector, e.g., a second AAV vector. In some embodiments, the first and second nucleic acid molecules are AAV vectors.

In some embodiments, (a), (b), and (c) are present on one nucleic acid molecule, e.g., one vector, e.g., one viral vector, e.g., an AAV vector. In some embodiments, the nucleic acid molecule is an AAV vector. In some embodiments, one of (a), (b), and (c) is present on a first nucleic acid molecule, e.g., a first vector, e.g., a first viral vector, e.g., a first AAV vector; and a second and third of (a), (b), and (c) is encoded on a second nucleic acid molecule, e.g., a second vector, e.g., a second vector, e.g., a second AAV vector. The first and second nucleic acid molecule may be AAV vectors.

In some embodiments, (a) is present on a first nucleic acid molecule, e.g., a first vector, e.g., a first viral vector, a first AAV vector; and (b) and (c) are present on a second nucleic acid molecule, e.g., a second vector, e.g., a second vector, e.g., a second AAV vector. The first and second nucleic acid molecule may be AAV vectors.

In some embodiments, (b) is present on a first nucleic acid molecule, e.g., a first vector, e.g., a first viral vector, e.g., a first AAV vector; and (a) and (c) are present on a second nucleic acid molecule, e.g., a second vector, e.g., a second vector, e.g., a second AAV vector. The first and second nucleic acid molecule may be AAV vectors.

In some embodiments, (c) is present on a first nucleic acid molecule, e.g., a first vector, e.g., a first viral vector, e.g., a first AAV vector; and (b) and (a) are present on a second nucleic acid molecule, e.g., a second vector, e.g., a second vector, e.g., a second AAV vector. The first and second nucleic acid molecule may be AAV vectors.

In some embodiments, each of (a), (b) and (c) are present on different nucleic acid molecules, e.g., different vectors, e.g., different viral vectors, e.g., different AAV vector. For example, (a) may be on a first nucleic acid molecule, (b) on a second nucleic acid molecule, and (c) on a third nucleic acid molecule. The first, second and third nucleic acid molecule may be AAV vectors.

AAV vectors may be formulated as AAV particles as described herein. In some embodiments, AAV particles comprise (i) an AAV polynucleotide construct (e.g., a recombinant AAV polynucleotide construct), and (ii) a capsid comprising capsid proteins. In some embodiments, an AAV polynucleotide construct comprises a polynucleotide sequence encoding a CRISPR/Cas12a effector protein or a characteristic portion thereof. In some embodiments, an AAV polynucleotide construct comprises a polynucleotide sequence encoding a gRNA molecule or a characteristic portion thereof.

In certain embodiments, the contacting comprises delivering to the cell the gRNA of (a) as an RNA, optionally the second gRNA of (c) as an RNA, and a nucleic acid composition that encodes a Cas12a effector protein or fusion protein of (b).

In some embodiments, a gRNA molecule as described herein and a Cas12a effector protein or fusion protein as described herein or a nucleic acid encoding the Cas12a effector protein or fusion protein, and optionally the gRNA molecule, and further, optionally, a second gRNA molecule, as described herein can be delivered to a cell via a lipid-based system. A lipid-based system can comprise any components and/or structures known in the art. In some embodiments, a lipid-based system is or comprises a lipid nanoparticle (LNP).

A CRISPR/Cas effector protein or fusion protein can be delivered to the cell as a protein or a nucleic acid encoding the protein, e.g., a DNA molecule or mRNA molecule. The guide molecule can be delivered as an RNA molecule or encoded by a DNA molecule. A CRISPR/Cas effector protein or fusion protein can also be delivered with a guide molecule as a ribonucleoprotein (RNP) and introduced into the cell via nucleofection (electroporation).

In some embodiments, the method of altering a cell, e.g., altering the structure, e.g., altering the sequence, of a target nucleic acid of a cell, comprising altering one or more target genes expressed by target cells as described herein. In some embodiments, the method of altering a cell comprises altering two or more target genes expressed by target cells as described herein. In some embodiments, the method of altering a cell comprises altering three or more target genes expressed by target cells as described herein. In some embodiments, the method of altering a cell comprises altering four or more target genes expressed by target cells as described herein. In some embodiments, the method of altering a cell comprises altering five or more target genes expressed by target cells as described herein. In some embodiments, the method of altering a cell comprises altering six or more target genes expressed by target cells as described herein. In certain embodiments, the method of altering a cell comprises altering seven or more target genes expressed by target cells as described herein. In certain embodiments, the method of altering a cell comprises altering each of a target gene as described herein.

In some embodiments, a contacting step comprises contacting the cell with a nucleic acid composition as described herein. In some embodiments, a contacting step comprises contacting the cell with a composition as described herein. In some embodiments, the composition is a ribonucleoprotein composition.

In some embodiments, a nucleic acid composition further comprises (c) a third nucleotide sequence that encodes a second gRNA molecule comprising a targeting domain that is complementary with a target domain from a target cell. In some embodiments, a second gRNA targets the same target position as the first gRNA molecule.

The presently disclosed subject matter further provides a reaction mixture comprising a, gRNA molecule as described herein, a nucleic acid composition as described herein, or a composition as described herein, and a cell, e.g., a cell from a subject who would benefit from one or more alteration at one or more cell target positions in the one or more target genes.

The presently disclosed subject matter further provides a kit comprising, (a) a gRNA molecule as described herein, or a nucleic acid composition that encodes the gRNA, and one or more of the following: (b) a Cas12a effector protein or fusion protein as described herein: (c) a second gRNA molecule as described herein.

Additionally, the presently disclosed subject matter provides a gRNA molecule as described herein for use in treating a disease, e.g., a cancer, in a subject. In some embodiments, the gRNA molecule is used in combination with (b) a Cas12a effector protein or fusion protein.

The presently disclosed subject matter further provides use of a gRNA molecule as described herein in the manufacture of a medicament for treating a disease, e.g., a cancer, in a subject. In certain embodiments, the medicament further comprises (b) a Cas12a effector protein or fusion protein.

A skilled person will understand that modulation of target site functionality may involve a CRISPR effector protein variant (such as for instance generation of a catalytically inactive or dead CRISPR effector) and/or functionalization (such as for instance fusion of the CRISPR effector with a heterologous functional domain, such as a deaminase), as described herein. Accordingly, in some embodiments the present disclosure relates to engineered compositions for site directed base editing comprising modified CRISPR effector protein and functional domain(s). In some embodiments, a functional domain comprises a deaminase or catalytic domain thereof, including a cytidine and/or adenine deaminase. Example functional domains suitable for use in the embodiments disclosed herein are discussed in further detail herein.

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially” of indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: AsCas12a Effector Protein Variants (Nucleases) with Increased Activity

The present example describes AsCas12a effector proteins (nucleases) comprising one or more substitutions at certain residues with increased activity compared to wild-type AsCas12a proteins and other AsCas12a proteins.

The disclosure contemplates that certain amino acid residues of an AsCas12a effector protein may be substituted (or mutated) to generate AsCas12a effector proteins with increased activity(ies). An AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety).

As described in Example 5, Q571K and C1003Y substitutions in Lb2Cas12a effector proteins increased activity compared to wild-type Lb2Cas12a. It is an insight of the present disclosure that these substitutions, alone or in combination with other mutations, are expected to confer increased activity in AsCas12a effector proteins.

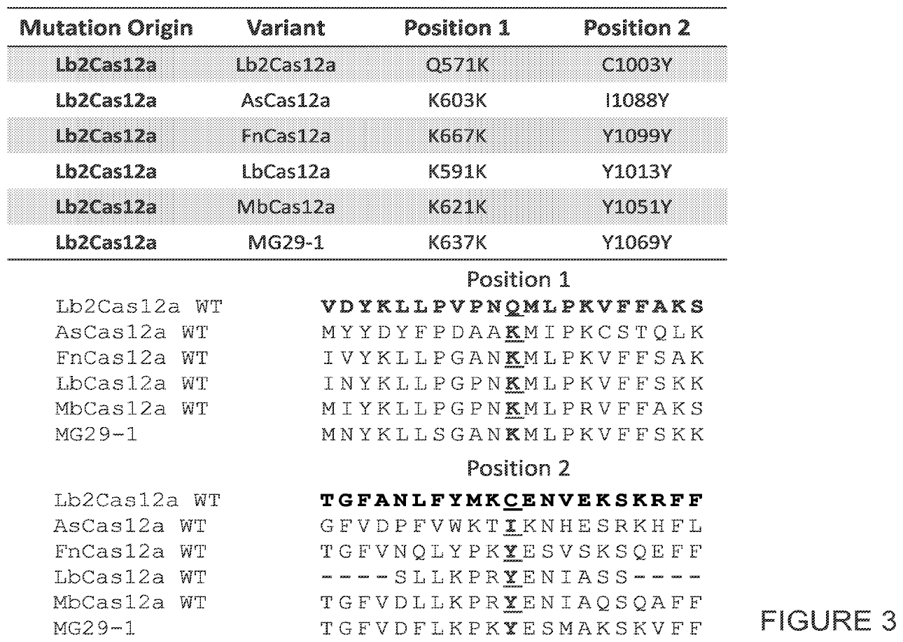

It was appreciated that amino acid sequences surrounding Lb2Cas12a Q571K and C1003Y mutations are conserved across Cas12a effector proteins. For example, FIG. 3 shows an alignment of wild-type AsCas12a (SEQ ID NO: 1) mapped to wild-type Lb2Cas12a (SEQ ID NO: 3) amino acid sequences surrounding these two mutations (Lb2Cas12a Q571K, Lb2Cas12a C1003Y) (see FIG. 3). After mapping, it was found that wild-type AsCas12a (SEQ ID NO: 1) also already contains a K amino acid at corresponding position of Q571K Lb2Cas12a (see FIG. 3). However, residue 1088 of AsCas12a is readily amenable to mutagenesis and can be mutated to I1088Y. Accordingly, the present example describes an AsCas12a effector protein having one or more amino acid substitutions corresponding to the group consisting of: M537R, F870L, and I1088Y (e.g., see SEQ ID NO: 12).

The present example also describes a variety of AsCas12a effector proteins comprising one of more mutations (e.g., E174R, S542R, and K548R, e.g., SEQ ID NO: 83) that exhibit higher activity compared to wild-type AsCas12a effector proteins (SEQ ID NO: 1).

The present example also describes a variety of AsCas12a effector proteins comprising one or more mutations that exhibit higher activity compared to wild-type AsCas12a effector proteins or AsCas12a effector proteins comprising SEQ ID NO: 6. This example refers to such exemplary AsCas12a effector proteins as “charge mutants”.

A variety of charge mutants were made and/or can be made by rational design as described herein (e.g., SEQ ID NOs: 7-10). For example, amino acid substitutions described by this example were designed as those residues that are spatially segregated from substitutions made in Exemplary AsCas12a variant 1 (SEQ ID NO: 6).

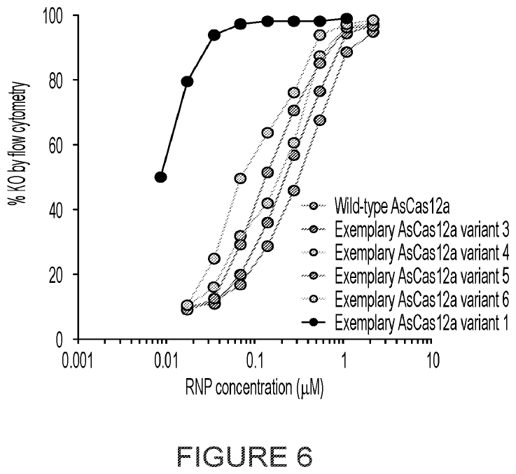

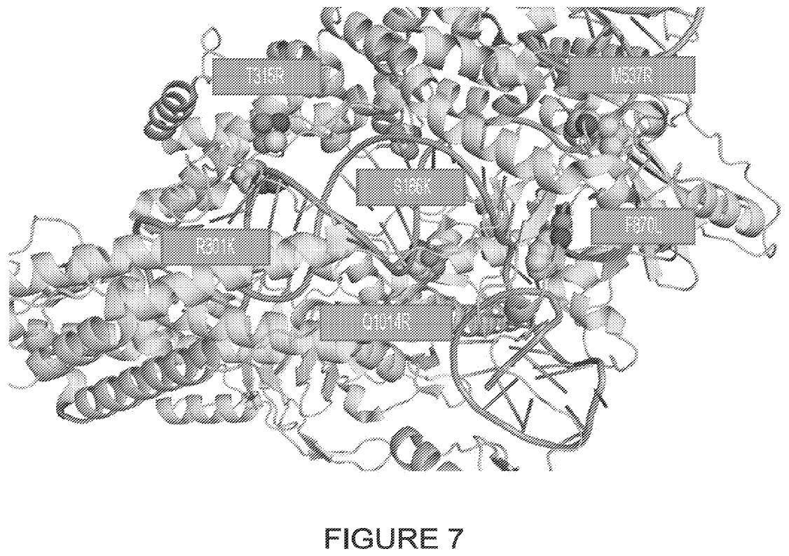

Charge mutants (e.g., Exemplary AsCas12a variant 3 (SEQ ID NO: 7), Exemplary AsCas12a variant 4 (SEQ ID NO: 8), Exemplary AsCas12a variant 5 (SEQ ID NO: 9), and Exemplary AsCas12a variant 6 (SEQ ID NO: 10), Exemplary AsCas12a variant 1 (SEQ ID NO: 6), and wild-type AsCas12a (SEQ ID NO: 1) were formulated as RNPs and administered to target cells at different concentrations to determine knock out efficiency of a target gene (TRAC) as determined by flow cytometry (see FIG. 6). Assessment of activity from each of the AsCas12a effector proteins shows improved knock out (KO) efficiency (%) compared to wild-type AsCas12a. It is contemplated that combining one or more mutations of Exemplary AsCas12a variant 1 (SEQ ID NO: 6) with one or more of amino acid substitutions made in Exemplary AsCas12a variant 3 (SEQ ID NO: 7), Exemplary AsCas12a variant 4 (SEQ ID NO: 8), Exemplary AsCas12a variant 5 (SEQ ID NO: 9), or Exemplary AsCas12a variant 6 (SEQ ID NO: 10), may further improve activity of Cas12a effector proteins described herein. It is also contemplated that making corresponding mutation(s) in highly conserved regions of Cas12a orthologues would confer increased activity.

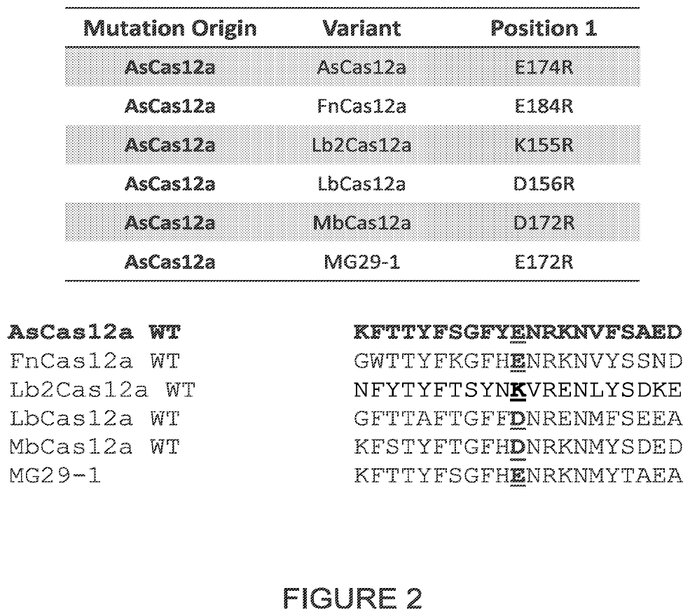

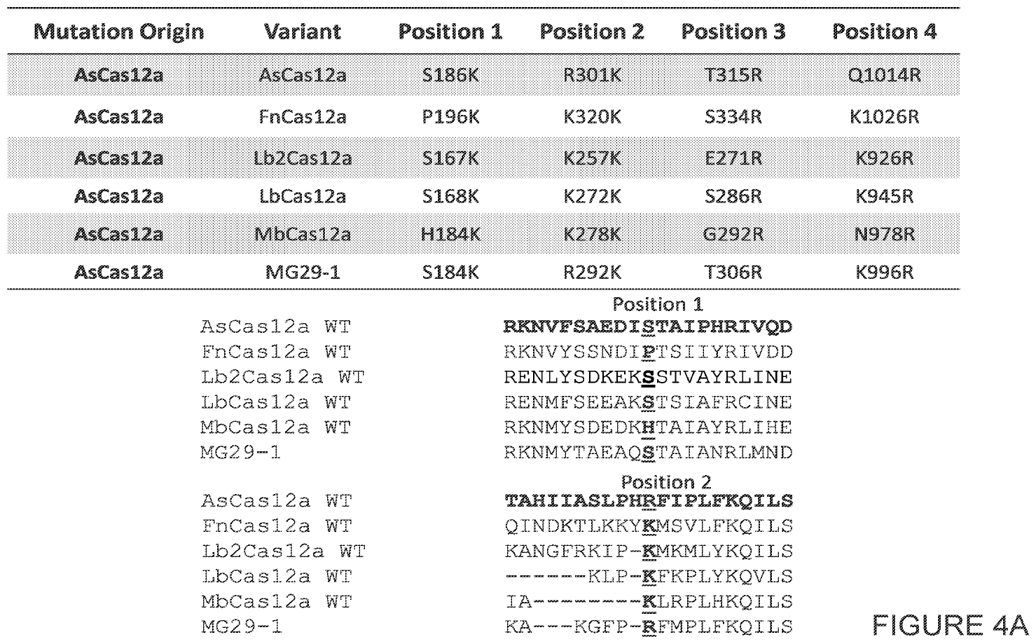

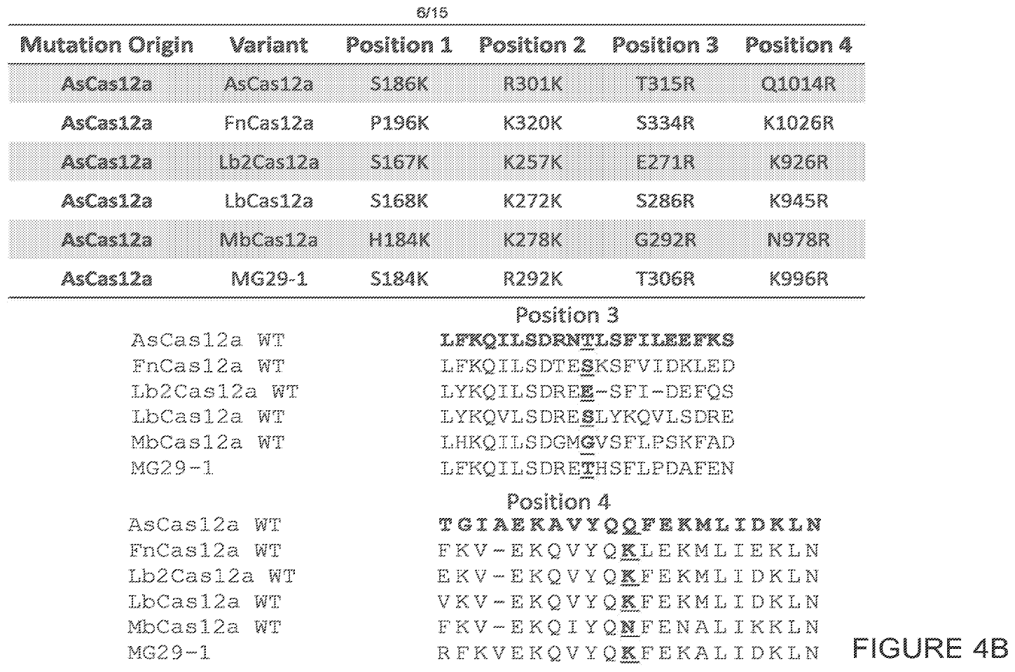

Additional combinations of amino acid substitutions (mutations) relative to exemplary AsCas12a wild-type amino acid sequence SEQ ID NO: 1 are shown in Table 6. Alignments of AsCas12a sequences and exemplary substitutions as described herein are provided in FIGS. 2, 3, and 4A-B. In some cases, an AsCas12a effector protein can comprise or consist of any of the combinations of amino acid substitutions present in Table 6 relative to an exemplary AsCas12a wild-type effector protein (SEQ ID NO: 1).

TABLE 6
AsCas12a Variants with substitutions relative to SEQ ID NO: 1

| | M537R | F870L | E174R | I1088Y | S186K | R301K | T315R | Q1014R |
|---|---|---|---|---|---|---|---|---|
| 1 | | | | X | | | | |
| 2 | | | X | X | | | | |
| 3 | X | | X | | | | | |
| 4 | X | | | X | | | | |
| 5 | | X | X | | | | | |
| 6 | | X | | X | | | | |
| 7 | X | X | X | | | | | |
| 8 | X | X | | X | | | | |
| 9 | X | X | X | X | | | | |
| 10 | | | | | X | | | |
| 11 | | | | | | X | | |
| 12 | | | | | | | X | |
| 13 | | | | | | | | X |
| 14 | | | | | X | X | | |
| 15 | | | | | X | | X | |
| 16 | | | | | X | | | X |
| 17 | | | | | | X | X | |
| 18 | | | | | | X | | X |
| 19 | | | | | | | X | X |
| 20 | | | | | X | X | X | |
| 21 | | | | | X | X | | X |
| 22 | | | | | X | | X | X |
| 23 | | | | | X | X | X | X |
| 24 | X | | | | X | | | |
| 25 | X | | | | | X | | |
| 26 | X | | | | | | X | |
| 27 | X | | | | | | | X |
| 28 | X | | | | X | X | | |
| 29 | X | | | | X | | X | |
| 30 | X | | | | X | | | X |
| 31 | X | | | | | X | X | |
| 32 | X | | | | | X | | X |
| 33 | X | | | | | | X | X |
| 34 | X | | | | X | X | X | |
| 35 | X | | | | X | X | | X |
| 36 | X | | | | X | | X | X |
| 37 | X | | | | X | X | X | X |
| 38 | X | | X | | X | X | X | X |
| 39 | X | | | X | X | X | X | X |
| 40 | X | | X | X | X | X | X | X |
| 41 | | X | | | X | | | |
| 42 | | X | | | | X | | |
| 43 | | X | | | | | X | |
| 44 | | X | | | | | | X |
| 45 | | X | | | X | X | | |
| 46 | | X | | | X | | X | |
| 47 | | X | | | X | | | X |
| 48 | | X | | | | X | X | |
| 49 | | X | | | | X | | X |
| 50 | | X | | | | | X | X |
| 51 | | X | | | X | X | X | |
| 52 | | X | | | X | X | | X |
| 53 | | X | | | X | | X | X |
| 54 | | X | | | X | X | X | X |
| 55 | | X | X | | X | X | X | X |
| 56 | | X | | X | X | X | X | X |
| 57 | | X | X | X | X | X | X | X |
| 58 | X | X | | | X | | | |
| 59 | X | X | | | | X | | |
| 60 | X | X | | | | | X | |
| 61 | X | X | | | | | | X |
| 62 | X | X | | | X | X | | |
| 63 | X | X | | | X | | X | |
| 64 | X | X | | | X | | | X |
| 65 | X | X | | | | X | X | |
| 66 | X | X | | | | X | | X |
| 67 | X | X | | | | | X | X |
| 68 | X | X | | | X | X | X | |
| 69 | X | X | | | X | X | | X |
| 70 | X | X | | | X | | X | X |
| 71 | X | X | | | X | X | X | X |
| 72 | X | X | X | | X | X | X | X |
| 73 | X | X | | X | X | X | X | X |
| 74 | X | X | X | X | X | X | X | X |

It is contemplated that amino acid substitutions that can be made are not limited by the present disclosure, and other substitutions can be incorporated into AsCas12a effector (nuclease) proteins.

### Example 2: AsCas12a Effector Protein Variants (Nickases) with Increased Activity

The present example describes AsCas12a effector proteins (nickases) comprising one or more substitutions at certain residues with increased activity compared to wild-type AsCas12a proteins.

An AsCas12a effector protein comprising two or more substitutions (e.g., K1000G and S1001G) has been demonstrated to result in a nickase version of wild-type AsCas12a (SEQ ID NO: 1). Additional combinations of these nickase-inducing mutations with amino acid substitutions (mutations) that increase activity relative to exemplary AsCas12a wild-type amino acid sequence SEQ ID NO: 1 are shown in Table 7. Alignments of sequences and exemplary substitutions as described herein are provided in FIGS. 2, 3, and 4A-B. In some cases, an AsCas12a effector protein can comprise or consist of any of the combinations of amino acid substitutions present in Table 7 relative to an exemplary AsCas12a wild-type effector protein (SEQ ID NO: 1).

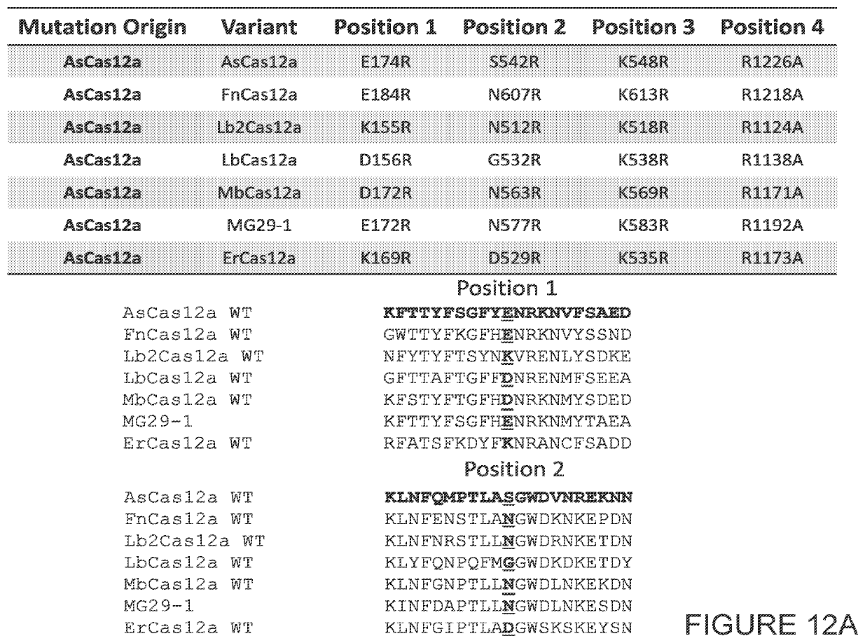

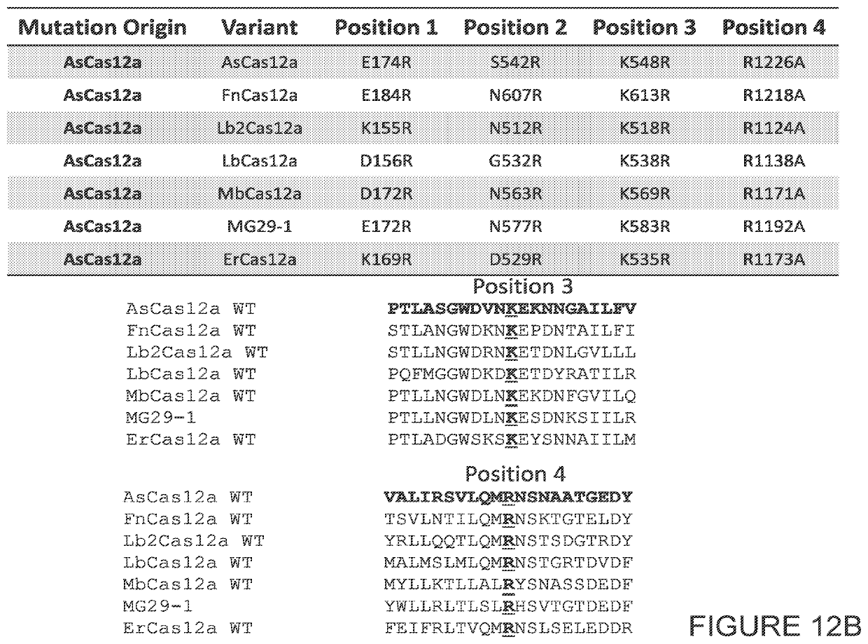

An AsCas12a effector protein comprising a substitution (e.g., R1226A) has been demonstrated to result in a nickase version of an AsCas12a effector protein (SEQ ID NO: 83). Additional combinations of this nickase-inducing mutation with amino acid substitutions (mutations) that increase activity relative to exemplary AsCas12a wild-type amino acid sequence (SEQ ID NO: 1) are shown in Table 7. Alignments of sequences and exemplary substitutions as described herein are provided in FIGS. 12A-B. In some cases, an AsCas12a effector protein can comprise or consist of any of the combinations of amino acid substitutions present in Table 7 relative to an exemplary AsCas12a wild-type effector protein (SEQ ID NO: 1).

TABLE 7
AsCas12a Variants with substitutions relative to SEQ ID NO: 1

| | M537R | F870L | E174R | I1088Y | S186K | R301K | T315R | Q1014R | K1000G | S1001G | R1226A |
|---|---|---|---|---|---|---|---|---|---|---|---|
| 1 | | | | | | | | | X | | |
| 2 | | | | | | | | | | X | |
| 3 | | | | | | | | | X | X | |
| 4 | X | | | | | | | | X | | |
| 5 | X | | | | | | | | | X | |
| 6 | | X | | | | | | | X | | |
| 7 | | X | | | | | | | | X | |
| 8 | X | X | | | | | | | X | | |
| 9 | X | X | | | | | | | | X | |
| 10 | X | X | | | | | | | X | X | |
| 11 | | | X | | | | | | X | | |
| 12 | | | X | | | | | | | X | |
| 13 | | | X | | | | | | X | X | |
| 14 | | | | X | | | | | X | | |
| 15 | | | | X | | | | | | X | |
| 16 | | | | X | | | | | X | X | |
| 17 | | | X | X | | | | | X | | |
| 18 | | | X | X | | | | | | X | |
| 19 | | | X | X | | | | | X | X | |
| 20 | X | | X | | | | | | X | | |
| 21 | X | | X | | | | | | | X | |
| 22 | X | | X | | | | | | X | X | |
| 23 | | X | X | | | | | | X | | |
| 24 | | X | X | | | | | | | X | |
| 25 | | X | X | | | | | | X | X | |
| 26 | X | X | X | | | | | | X | | |
| 27 | X | X | X | | | | | | | X | |
| 28 | X | X | X | | | | | | X | X | |
| 29 | X | | | X | | | | | X | | |
| 30 | X | | | X | | | | | | X | |
| 31 | X | | | X | | | | | X | X | |
| 32 | | X | | X | | | | | X | | |
| 33 | | X | | X | | | | | | X | |
| 34 | | X | | X | | | | | X | X | |
| 35 | X | X | | X | | | | | X | | |
| 36 | X | X | | X | | | | | | X | |
| 37 | X | X | | X | | | | | X | X | |
| 38 | X | X | X | X | | | | | X | | |
| 39 | X | X | X | X | | | | | | X | |
| 40 | X | X | X | X | | | | | X | X | |
| 41 | | | | | X | | | | X | | |
| 42 | | | | | X | | | | | X | |
| 43 | | | | | X | | | | X | X | |
| 44 | | | | | | X | | | X | | |
| 45 | | | | | | X | | | | X | |
| 46 | | | | | | X | | | X | X | |
| 47 | | | | | | | X | | X | | |
| 48 | | | | | | | X | | | X | |
| 49 | | | | | | | X | | X | X | |
| 50 | | | | | | | | X | X | | |
| 51 | | | | | | | | X | | X | |
| 52 | | | | | | | | X | X | X | |
| 53 | | | | | X | X | | | X | | |
| 54 | | | | | X | X | | | | X | |
| 55 | | | | | X | X | | | X | X | |
| 56 | | | | | X | | X | | X | | |
| 57 | | | | | X | | X | | | X | |
| 58 | | | | | X | | X | | X | X | |
| 59 | | | | | X | | | X | X | | |
| 60 | | | | | X | | | X | | X | |
| 61 | | | | | X | | | X | X | X | |
| 62 | | | | | | X | X | | X | | |
| 63 | | | | | | X | X | | | X | |
| 64 | | | | | | X | X | | X | X | |
| 65 | | | | | | X | | X | X | | |
| 66 | | | | | | X | | X | | X | |
| 67 | | | | | | X | | X | X | X | |
| 68 | | | | | | | X | X | X | | |
| 69 | | | | | | | X | X | | X | |
| 70 | | | | | | | X | X | X | X | |
| 71 | | | | | X | X | X | | X | | |
| 72 | | | | | X | X | X | | | X | |
| 73 | | | | | X | X | X | | X | X | |
| 74 | | | | | X | X | | X | X | | |
| 75 | | | | | X | X | | X | | X | |
| 76 | | | | | X | X | | X | X | X | |
| 77 | | | | | X | | X | X | X | | |
| 78 | | | | | X | | X | X | | X | |
| 79 | | | | | X | | X | X | X | X | |
| 80 | | | | | X | X | X | X | X | | |
| 81 | | | | | X | X | X | X | | X | |
| 82 | | | | | X | X | X | X | X | X | |
| 83 | X | X | | | X | | | | X | | |
| 84 | X | X | | | X | | | | | X | |
| 85 | X | X | | | X | | | | X | X | |
| 86 | X | X | | | | X | | | X | | |
| 87 | X | X | | | | X | | | | X | |
| 88 | X | X | | | | X | | | X | X | |
| 89 | X | X | | | | | X | | X | | |
| 90 | X | X | | | | | X | | | X | |
| 91 | X | X | | | | | X | | X | X | |
| 92 | X | X | | | | | | X | X | | |
| 93 | X | X | | | | | | X | | X | |
| 94 | X | X | | | | | | X | X | X | |
| 95 | X | X | | | X | X | | | X | | |
| 96 | X | X | | | X | X | | | | X | |
| 97 | X | X | | | X | X | | | X | X | |
| 98 | X | X | | | X | | X | | X | | |
| 99 | X | X | | | X | | X | | | X | |
| 100 | X | X | | | X | | X | | X | X | |
| 101 | | X | | | X | | | X | X | | |
| 102 | X | X | | | X | | | X | | X | |
| 103 | X | X | | | X | | | X | X | X | |
| 104 | X | X | | | | X | X | | X | | |
| 105 | X | X | | | | X | X | | | X | |
| 106 | X | X | | | | X | X | | X | X | |
| 107 | X | X | | | | X | | X | X | | |
| 108 | X | X | | | | X | | X | | X | |
| 109 | X | X | | | | X | | X | X | X | |
| 110 | X | X | | | | | X | X | X | | |
| 111 | X | X | | | | | X | X | | X | |
| 112 | X | X | | | | | X | X | X | X | |
| 113 | X | X | | | X | X | X | | X | | |
| 114 | X | X | | | X | X | X | | | X | |
| 115 | X | X | | | X | X | X | | X | X | |
| 116 | X | X | | | X | X | | X | X | | |
| 117 | X | X | | | X | X | | X | | X | |
| 118 | X | X | | | X | X | | X | X | X | |
| 119 | X | X | | | X | | X | X | X | | |
| 120 | X | X | | | X | | X | X | | X | |
| 121 | X | X | | | X | | X | X | X | X | |
| 122 | X | X | | | X | X | X | X | X | | |
| 123 | X | X | | | X | X | X | X | | X | |
| 124 | X | X | | | X | X | X | X | X | X | |
| 125 | X | X | X | | X | X | X | X | X | | |
| 126 | X | X | X | | X | X | X | X | | X | |
| 127 | X | X | X | | X | X | X | X | X | X | |
| 128 | X | X | | X | X | X | X | X | X | | |
| 129 | X | X | | X | X | X | X | X | | X | |
| 130 | X | X | | X | X | X | X | X | X | X | |
| 131 | X | X | X | X | X | X | X | X | X | | |
| 132 | X | X | X | X | X | X | X | X | | X | |
| 133 | X | X | X | X | X | X | X | X | X | X | |
| 134 | X | | | | X | | | | X | | |
| 135 | X | | | | X | | | | | X | |
| 136 | X | | | | X | | | | X | X | |
| 137 | X | | | | | X | | | X | | |
| 138 | X | | | | | X | | | | X | |
| 139 | X | | | | | X | | | X | X | |
| 140 | X | | | | | | X | | X | | |
| 141 | X | | | | | | X | | | X | |
| 142 | X | | | | | | X | | X | X | |
| 143 | X | | | | | | | X | X | | |
| 144 | X | | | | | | | X | | X | |
| 145 | X | | | | | | | X | X | X | |
| 146 | X | | | | X | X | | | X | | |
| 147 | X | | | | X | X | | | | X | |
| 148 | X | | | | X | X | | | X | X | |
| 149 | X | | | | X | | X | | X | | |
| 150 | X | | | | X | | X | | | X | |
| 151 | X | | | | X | | X | | X | X | |
| 152 | X | | | | X | | | X | X | | |
| 153 | X | | | | X | | | X | | X | |
| 154 | X | | | | X | | | X | X | X | |
| 155 | X | | | | | X | X | | X | | |
| 156 | X | | | | | X | X | | | X | |
| 157 | X | | | | | X | X | | X | X | |
| 158 | X | | | | | X | | X | X | | |
| 159 | X | | | | | | | X | | X | |
| 160 | X | | | | | X | | X | X | X | |
| 161 | X | | | | | | X | X | X | | |
| 162 | X | | | | | | X | X | | X | |
| 163 | X | | | | | | X | X | X | X | |
| 164 | X | | | | X | X | X | | X | | |
| 165 | X | | | | X | X | X | | | X | |
| 166 | X | | | | X | X | X | | X | X | |
| 167 | X | | | | X | X | | X | X | | |
| 168 | X | | | | X | X | | X | | X | |
| 169 | X | | | | X | X | | X | X | X | |
| 170 | X | | | | X | | X | X | X | | |
| 171 | X | | | | X | | X | X | | X | |
| 172 | X | | | | X | | X | X | X | X | |
| 173 | X | | | | X | X | X | X | X | | |
| 174 | X | | | | X | X | X | X | | X | |
| 175 | X | | | | X | X | X | X | X | X | |
| 176 | X | | X | | X | X | X | X | X | | |
| 177 | X | | X | | X | X | X | X | | X | |
| 178 | X | | X | | X | X | X | X | X | X | |
| 179 | X | | | X | X | X | X | X | X | | |
| 180 | X | | | X | X | X | X | X | | X | |
| 181 | X | | | X | X | X | X | X | X | X | |
| 182 | X | | X | X | X | X | X | X | X | | |
| 183 | X | | X | X | X | X | X | X | | X | |
| 184 | X | | X | X | X | X | X | X | X | X | |
| 185 | | X | | | X | | | | X | | |
| 186 | | X | | | X | | | | | X | |
| 187 | | X | | | X | | | | X | X | |
| 188 | | X | | | | X | | | X | | |
| 189 | | X | | | | X | | | | X | |
| 190 | | X | | | | X | | | X | X | |
| 191 | | X | | | | | X | | X | | |
| 192 | | X | | | | | X | | | X | |
| 193 | | X | | | | | X | | X | X | |
| 194 | | X | | | | | | X | X | | |
| 195 | | X | | | | | | X | | X | |
| 196 | | X | | | | | | X | X | X | |
| 197 | | X | | | X | X | | | X | | |
| 198 | | X | | | X | X | | | | X | |
| 199 | | X | | | X | X | | | X | X | |
| 200 | | X | | | X | | X | | X | | |
| 201 | | X | | | X | | X | | | X | |
| 202 | | X | | | X | | X | | X | X | |
| 203 | | X | | | X | | | X | X | | |
| 204 | | X | | | X | | | X | | X | |
| 205 | | X | | | X | | | X | X | X | |
| 206 | | X | | | | X | X | | X | | |
| 207 | | X | | | | X | X | | | X | |
| 208 | | X | | | | X | X | | X | X | |
| 209 | | X | | | | X | | X | X | | |
| 210 | | X | | | | X | | X | | X | |
| 211 | | X | | | | X | | X | X | X | |
| 212 | | X | | | | | X | X | X | | |
| 213 | | X | | | | | X | X | | X | |
| 214 | | X | | | | | X | X | X | X | |
| 215 | | X | | | X | X | X | | X | | |
| 216 | | X | | | X | X | X | | | X | |
| 217 | | X | | | X | X | X | | X | X | |
| 218 | | X | | | X | X | | X | X | | |
| 219 | | X | | | X | X | | X | | X | |
| 220 | | X | | | X | X | | X | X | X | |
| 221 | | X | | | X | | X | X | X | | |
| 222 | | X | | | X | | X | X | | X | |
| 223 | | X | | | X | | X | X | X | X | |
| 224 | | X | | | X | X | X | X | X | | |
| 225 | | X | | | X | X | X | X | | X | |
| 226 | | X | | | X | X | X | X | X | X | |
| 227 | | X | X | | X | X | X | X | X | | |
| 228 | | X | X | | X | X | X | X | | X | |
| 229 | | X | X | | X | X | X | X | X | X | |
| 230 | | X | | X | X | X | X | X | X | | |
| 231 | | X | | X | X | X | X | X | | X | |
| 232 | | X | | X | X | X | X | X | X | X | |
| 233 | | X | X | X | X | X | X | X | X | | |
| 234 | | X | X | X | X | X | X | X | | X | |
| 235 | | X | X | X | X | X | X | X | X | X | |
| 236 | | | | | | | | | | | X |
| 237 | X | | | | | | | | | | X |
| 238 | | X | | | | | | | | | X |
| 239 | X | X | | | | | | | | | X |
| 240 | | | X | | | | | | | | X |
| 241 | | | | X | | | | | | | X |
| 242 | | | X | X | | | | | | | X |
| 243 | X | | X | | | | | | | | X |
| 244 | | X | X | | | | | | | | X |
| 245 | X | X | X | | | | | | | | X |
| 246 | X | | | X | | | | | | | X |
| 247 | | X | | X | | | | | | | X |
| 248 | X | X | | X | | | | | | | X |
| 249 | X | X | X | X | | | | | | | X |
| 250 | | | | | X | | | | | | X |
| 251 | | | | | | X | | | | | X |
| 252 | | | | | | | X | | | | X |
| 253 | | | | | | | | X | | | X |
| 254 | | | | | X | X | | | | | X |
| 255 | | | | | X | | X | | | | X |
| 256 | | | | | X | | | X | | | X |
| 257 | | | | | | X | X | | | | X |
| 258 | | | | | | X | | X | | | X |
| 259 | | | | | | | X | X | | | X |
| 260 | | | | | X | X | X | | | | X |
| 261 | | | | | X | X | | X | | | X |
| 262 | | | | | X | | X | X | | | X |
| 263 | | | | | X | X | X | X | | | X |
| 264 | X | X | | | X | | | | | | X |
| 265 | X | X | | | | X | | | | | X |
| 266 | X | X | | | | | X | | | | X |
| 267 | X | X | | | | | | X | | | X |
| 268 | X | X | | | X | X | | | | | X |
| 269 | X | X | | | X | | X | | | | X |
| 270 | X | X | | | X | | | X | | | X |
| 271 | X | X | | | | X | X | | | | X |
| 272 | X | X | | | | X | | X | | | X |
| 273 | X | X | | | | | X | X | | | X |
| 274 | X | X | | | X | X | X | | | | X |
| 275 | X | X | | | X | X | | X | | | X |
| 276 | X | X | | | X | | X | X | | | X |
| 277 | X | X | | | X | X | X | X | | | X |
| 278 | X | X | X | | X | X | X | X | | | X |
| 279 | X | X | | X | X | X | X | X | | | X |
| 280 | X | X | X | X | X | X | X | X | | | X |
| 281 | X | | | | X | | | | | | |
| 282 | X | | | | | X | | | | | X |
| 283 | X | | | | | | X | | | | X |
| 284 | X | | | | | | | X | | | X |
| 285 | X | | | | X | X | | | | | X |
| 286 | X | | | | X | | X | | | | X |
| 287 | X | | | | X | | | X | | | X |
| 288 | X | | | | | X | X | | | | X |
| 289 | X | | | | | X | | X | | | X |
| 290 | X | | | | | | X | X | | | X |
| 291 | X | | | | X | X | X | | | | X |
| 292 | X | | | | X | X | | X | | | X |
| 293 | X | | | | X | | X | X | | | X |
| 294 | X | | | | X | X | X | X | | | X |
| 295 | X | | X | | X | X | X | X | | | X |
| 296 | X | | | X | X | X | X | X | | | X |
| 297 | X | | X | X | X | X | X | X | | | X |
| 298 | | X | | | X | | | | | | X |
| 299 | | X | | | | X | | | | | X |
| 300 | | X | | | | | X | | | | X |
| 301 | | X | | | | | | X | | | X |
| 302 | | X | | | X | X | | | | | X |
| 303 | | X | | | X | | X | | | | X |
| 304 | | X | | | X | | | X | | | X |
| 305 | | X | | | | X | X | | | | X |
| 306 | | X | | | | X | | X | | | X |
| 307 | | X | | | | | X | X | | | X |
| 308 | | X | | | X | X | X | | | | X |
| 309 | | X | | | X | X | | X | | | X |
| 310 | | X | | | X | | X | X | | | X |
| 311 | | X | | | X | X | X | X | | | X |
| 312 | | X | X | | X | X | X | X | | | X |
| 313 | | X | | X | X | X | X | X | | | X |
| 314 | | X | X | X | X | X | X | X | | | X |

In some embodiments, an AsCas12a effector protein can comprise a combination of mutations at one or more positions including E174, S542, K548, and R1226. In some embodiments, an AsCas12a effector protein can comprise a combination of E174R, S542R, K548R, and R1226A mutations.

It is contemplated that amino acid substitutions that can be made are not limited by the present disclosure, and other substitutions can be incorporated into Cas12a effector (nickase) proteins.

### Example 3: FnCas12a Effector Protein Variants (Nucleases) with Increased Activity

The present example describes FnCas12a effector proteins (nucleases) comprising one or more substitutions at certain residues with increased activity compared to wild-type FnCas12a proteins.

The disclosure contemplates that certain amino acid residues of an FnCas12a effector protein may be substituted (or mutated) to generate FnCas12a effector proteins with increased activity(ies). For example, the present disclosure describes that N602 and/or F879 residues of an amino acid sequence provided in SEQ ID NO: 2 can be substituted (or mutated). The present disclosure also describes that other residues of an amino acid sequence provided in SEQ ID NO: 2 can be substituted (or mutated) in various combinations.

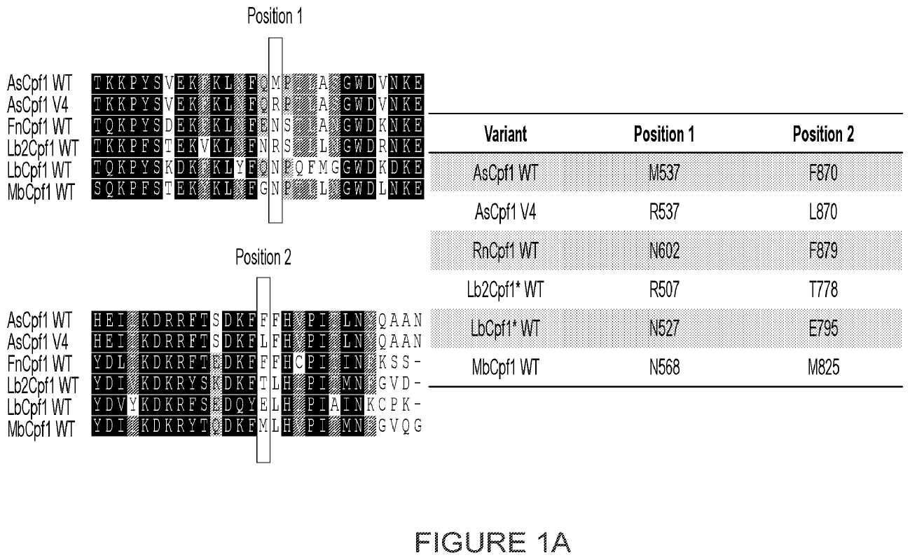

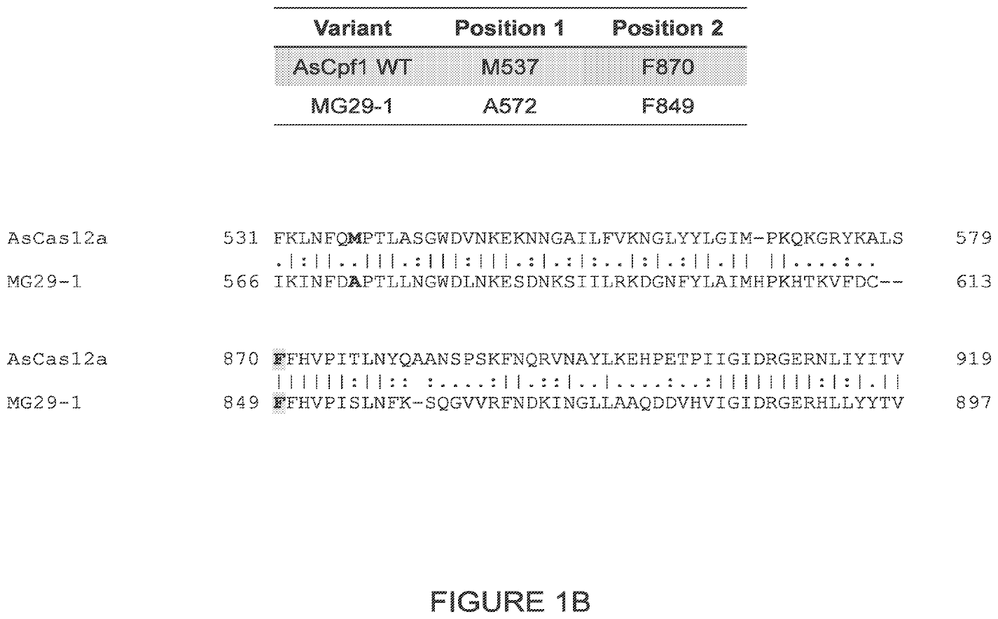

As described in Example 1, an AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety). It is an insight of the present disclosure that FnCas12a effector proteins and AsCas12a effector proteins share sequence conservation of domains (see FIG. 1A). Without wishing to be bound by any theory, the present example provides FnCas12a effector proteins with increased activity that are achieved by mutating residues in these conserved regions. For example, without wishing to be bound by any theory, the present example describes that mutating residues N602 and/or F879 of FnCas12a is expected to produce FnCas12a effector proteins with higher activity compared to wild-type FnCas12a effector proteins. In particular, mutating residues N602 and/or F879 of FnCas12a to have N602R and/or F879L mutations, which correspond to M537R and F870L in an exemplary AsCas12a protein (SEQ ID NO: 1), is expected to result in a higher activity FnCas12a relative to the wild-type protein.

Additional combinations of amino acid substitutions (mutations) relative to exemplary FnCas12a wild-type amino acid sequence SEQ ID NO: 2 are shown in Table 8. Alignments of FnCas12a sequences with corresponding AsCas12a or Lb2Cas12a sequences and exemplary substitutions as described herein are provided in FIGS. 2, 3, and 4A-B. In some cases, an FnCas12a effector protein can comprise or consist of any of the combinations of amino acid substitutions present in Table 8 relative to an exemplary FnCas12a wild-type effector protein (SEQ ID NO: 2).

TABLE 8
FnCas12a Variants with substitutions relative to SEQ ID NO: 2

| | N602R | F879L | E184R | P196K | S334R | K1026R |
|---|---|---|---|---|---|---|
| 1 | X | X | | | | |
| 2 | | | X | | | |
| 3 | X | | X | | | |
| 4 | | X | X | | | |
| 5 | X | X | X | | | |
| 6 | | | | X | | |
| 7 | | | | | X | |
| 8 | | | | | | X |
| 9 | | | | X | X | |
| 10 | | | | X | | X |
| 11 | | | | | X | X |
| 12 | | | | X | X | X |
| 13 | X | X | | X | | |
| 14 | X | X | | | X | |
| 15 | X | X | | | | X |
| 16 | X | X | | X | X | |
| 17 | X | X | | X | | X |
| 18 | X | X | | | X | X |
| 19 | X | X | | X | X | X |
| 20 | X | X | X | X | X | X |
| 21 | X | X | X | X | X | X |
| 22 | X | | | X | | |
| 23 | X | | | | X | |
| 24 | X | | | | | X |
| 25 | X | | | X | X | |
| 26 | X | | | X | | X |
| 27 | X | | | | X | X |
| 28 | X | | | X | X | X |
| 29 | X | | X | X | X | X |
| 30 | X | | X | X | X | X |
| 31 | | X | | X | | |
| 32 | | X | | | X | |
| 33 | | X | | | | X |
| 34 | | X | | X | X | |
| 35 | | X | | X | | X |
| 36 | | X | | | X | X |
| 37 | | X | | X | X | X |
| 38 | | X | X | X | X | X |
| 39 | | X | X | X | X | X |

It is contemplated that amino acid substitutions that can be made are not limited by the present disclosure, and other substitutions can be incorporated into AsCas12a effector (nuclease) proteins.

### Example 4: FnCas12a Effector Protein Variants (Nickases) with Increased Activity

The present example describes FnCas12a effector proteins (nickases) comprising one or more substitutions at certain residues with increased activity compared to wild-type FnCas12a proteins.

The disclosure contemplates that certain amino acid residues of an FnCas12a effector protein may be substituted (or mutated) to generate FnCas12a effector proteins that are nickases, optionally with increased activity(ies). For example, the present disclosure describes that K1013 and/or R1014 residues of an amino acid sequence provided in SEQ ID NO: 2 can be substituted (or mutated).

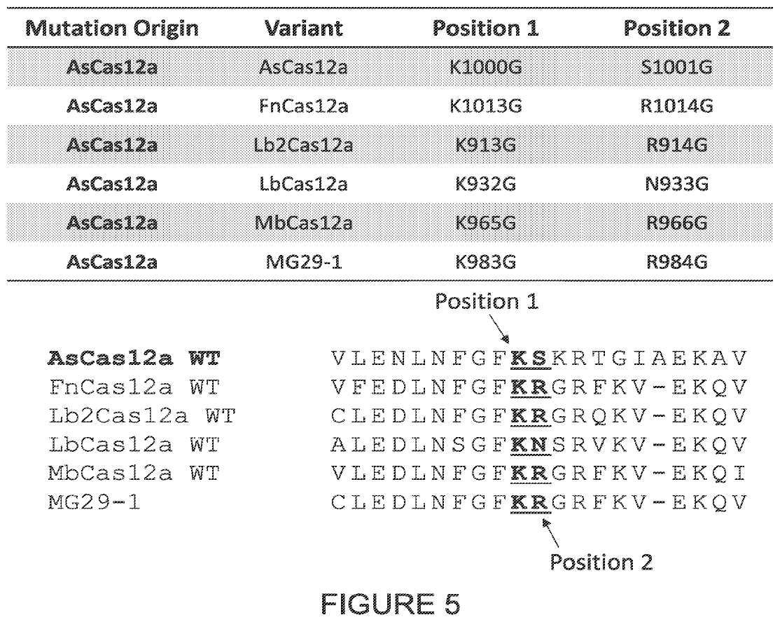

An AsCas12a effector protein comprising two or more substitutions (e.g., K1000G and S1001G) has been demonstrated to result in a nickase version of wild-type AsCas12a (SEQ ID NO: 1). It is an insight of the present disclosure that FnCas12a effector proteins and AsCas12a effector proteins share sequence conservation around these positions (see FIG. 5). Without wishing to be bound by any theory, the present example provides FnCas12a effector proteins with nickase activity by mutating residues in these conserved regions. For example, without wishing to be bound by any theory, the present example describes that mutating residues K1013 and/or R1014 of FnCas12a is expected to produce FnCas12a effector proteins with nickase activity. In particular, mutations residues K1013 and/or R1014 of FnCas12a to have K1013G and/or R1014G, which correspond to K1000G and S1001G in an exemplary AsCas12a effector protein (SEQ ID NO: 1), is expected to result in an FnCas12a effector protein with nickase activity.

An AsCas12a effector protein comprising a substitution (e.g., R1226A) has been demonstrated to result in a nickase version of an AsCas12a effector protein (SEQ ID NO: 83). It is an insight of the present disclosure that FnCas12a effector proteins and AsCas12a effector proteins share sequence conservation around this position (see FIG. 5). Without wishing to be bound by any theory, the present example provides FnCas12a effector proteins with nickase activity by mutating residues in these conserved regions. For example, without wishing to be bound by any theory, the present example describes that mutating a residue R1218 of FnCas12a is expected to produce FnCas12a effector proteins with nickase activity. In particular, mutation of residue R1218 of FnCas12a to have R1218A, which correspond to R1226A in an exemplary AsCas12a effector protein (SEQ ID NO: 1), is expected to result in an FnCas12a effector protein with nickase activity.

Additional combinations of these nickase-inducing mutations with amino acid substitutions (mutations) that increase activity relative to exemplary FnCas12a wild-type amino acid sequence SEQ ID NO: 2 are shown in Table 9. Alignments of FnCas12a sequences with corresponding AsCas12a or Lb2Cas12a sequences and exemplary substitutions as described herein are provided in FIGS. 2, 3, and 4A-B. In some cases, an FnCas12a effector protein can comprise or consist of any of the combinations of amino acid substitutions present in Table 9 relative to an exemplary FnCas12a wild-type effector protein (SEQ ID NO: 2).

TABLE 9
FnCas12a Variants with substitutions relative to SEQ ID NO: 2

| | N602R | F879L | E184R | P196K | S334R | K1026R | K1013G | R1014G | R1218A |
|---|---|---|---|---|---|---|---|---|---|
| 1 | | | | | | | X | | |
| 2 | | | | | | | | X | |
| 3 | | | | | | | X | X | |
| 4 | X | | | | | | X | | |
| 5 | X | | | | | | | X | |
| 6 | | X | | | | | X | | |
| 7 | | X | | | | | | X | |
| 8 | X | X | | | | | X | | |
| 9 | X | X | | | | | | X | |
| 10 | X | X | | | | | X | X | |
| 11 | | | X | | | | X | | |
| 12 | | | X | | | | | X | |
| 13 | | | X | | | | X | X | |
| 14 | X | | X | | | | X | | |
| 15 | X | | X | | | | | X | |
| 16 | X | | X | | | | X | X | |
| 17 | | X | X | | | | X | | |
| 18 | | X | X | | | | | X | |
| 19 | | X | X | | | | X | X | |
| 20 | X | X | X | | | | X | | |
| 21 | X | X | X | | | | | X | |
| 22 | X | X | X | | | | X | X | |
| 23 | | | | X | | | X | | |
| 24 | | | | X | | | | X | |
| 25 | | | | X | | | X | X | |
| 26 | | | | | X | | X | | |
| 27 | | | | | X | | | X | |
| 28 | | | | | X | | X | X | |
| 29 | | | | | | X | X | | |
| 30 | | | | | | X | | X | |
| 31 | | | | | | X | X | X | |
| 32 | | | | X | X | | X | | |
| 33 | | | | X | X | | | X | |
| 34 | | | | X | X | | X | X | |
| 35 | | | | X | | X | X | | |
| 36 | | | | X | | X | | X | |
| 37 | | | | X | | X | X | X | |
| 38 | | | | | X | X | X | | |
| 39 | | | | | X | X | | X | |
| 40 | | | | | X | X | X | X | |
| 41 | | | | X | X | X | X | | |
| 42 | | | | X | X | X | | X | |
| 43 | | | | X | X | X | X | X | |
| 44 | X | | | X | | | X | | |
| 45 | X | | | X | | | | X | |
| 46 | X | | | X | | | X | X | |
| 47 | X | | | | X | | X | | |
| 48 | X | | | | X | | | X | |
| 49 | X | | | | X | | X | X | |
| 50 | X | | | | | X | X | | |
| 51 | X | | | | | X | | X | |
| 52 | X | | | | | X | X | X | |
| 53 | X | | | X | X | | X | | |
| 54 | X | | | X | X | | | X | |
| 55 | X | | | X | X | | X | X | |
| 56 | X | | | X | | X | X | | |
| 57 | X | | | X | | X | | X | |
| 58 | X | | | X | | X | X | X | |
| 59 | X | | | | X | X | X | | |
| 60 | X | | | | X | X | | X | |
| 61 | X | | | | X | X | X | X | |
| 62 | X | | | X | X | X | X | | |
| 63 | X | | | X | X | X | | X | |
| 64 | X | | | X | X | X | X | X | |
| 65 | X | | X | X | X | X | X | X | |
| 66 | | X | | X | | | X | | |
| 67 | | X | | X | | | | X | |
| 68 | | X | | X | | | X | X | |
| 69 | | X | | | X | | X | | |
| 70 | | X | | | X | | | X | |
| 71 | | X | | | X | | X | X | |
| 72 | | X | | | | X | X | | |
| 73 | | X | | | | X | | X | |
| 74 | | X | | | | X | X | X | |
| 75 | | X | | X | X | | X | | |
| 76 | | X | | X | X | | | X | |
| 77 | | X | | X | X | | X | X | |
| 78 | | X | | X | | X | X | | |
| 79 | | X | | X | | X | | X | |
| 80 | | X | | X | | X | X | X | |
| 81 | | X | | | X | X | X | | |
| 82 | | X | | | X | X | | X | |
| 83 | | X | | | X | X | X | X | |
| 84 | | X | | X | X | X | X | | |
| 85 | | X | | X | X | X | | X | |
| 86 | | X | | X | X | X | X | X | |
| 87 | | X | X | X | X | X | X | X | |
| 88 | X | X | | X | | | X | | |
| 89 | X | X | | X | | | | X | |
| 90 | X | X | | X | | | X | X | |
| 91 | X | X | | | X | | X | | |
| 92 | X | X | | | X | | | X | |
| 93 | X | X | | | X | | X | X | |
| 94 | X | X | | | | X | X | | |
| 95 | X | X | | | | X | | X | |
| 96 | X | X | | | | X | X | X | |
| 97 | X | X | | X | X | | X | | |
| 98 | X | X | | X | X | | | X | |
| 99 | X | X | | X | X | | X | X | |
| 100 | X | X | | X | | X | X | | |
| 101 | X | X | | X | | X | | X | |
| 102 | X | X | | X | | X | X | X | |
| 103 | X | X | | | X | X | X | | |
| 104 | X | X | | | X | X | | X | |
| 105 | X | X | | | X | X | X | X | |
| 106 | X | X | | X | X | X | X | | |
| 107 | X | X | | X | X | X | | X | |
| 108 | X | X | | X | X | X | X | X | |
| 109 | X | X | X | X | X | X | X | X | |
| 110 | | | | | | | | | X |
| 111 | X | | | | | | | | X |
| 112 | | X | | | | | | | X |
| 113 | X | X | | | | | | | X |
| 114 | | | X | | | | | | X |
| 115 | X | | X | | | | | | X |
| 116 | | X | X | | | | | | X |
| 117 | X | X | X | | | | | | X |
| 118 | | | | X | | | | | X |
| 119 | | | | | X | | | | X |
| 120 | | | | | | X | | | X |
| 121 | | | | X | X | | | | X |
| 122 | | | | X | | X | | | X |
| 123 | | | | | X | X | | | X |
| 124 | | | | X | X | X | | | X |
| 125 | X | | | X | | | | | X |
| 126 | X | | | | X | | | | X |
| 127 | X | | | | | X | | | X |
| 128 | X | | | X | X | | | | X |
| 129 | X | | | X | | X | | | X |
| 130 | X | | | | X | X | | | X |
| 131 | X | | | X | X | X | | | X |
| 132 | X | | X | X | X | X | | | X |
| 133 | | X | | X | | | | | X |
| 134 | | X | | | X | | | | X |
| 135 | | X | | | | X | | | X |
| 136 | | X | | X | X | | | | X |
| 137 | | X | | X | | X | | | X |
| 138 | | X | | | X | X | | | X |
| 139 | | X | | X | X | X | | | X |
| 140 | | X | X | X | X | X | | | X |
| 141 | X | X | | X | | | | | X |
| 142 | X | X | | | X | | | | X |
| 143 | X | X | | | | X | | | X |
| 144 | X | X | | X | X | | | | X |
| 145 | X | X | | X | | X | | | X |
| 146 | X | X | | | X | X | | | X |
| 147 | X | X | | X | X | X | | | X |
| 148 | X | X | X | X | X | X | | | X |

In some embodiments, an FnCas12a effector protein can comprise a combination of the R1218A mutation with mutations of residues E184, N607, and K613 of FnCas12a to produce effector proteins with increased nickase activity. In particular, mutation of residues E184, N607, and K613 of FnCas12a to have E184R, N607R, and K613R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into Cas12a effector (nickase) proteins.

### Example 5: Lb2Cas12a Effector Protein Variants (Nucleases) with Increased Activity

The present example describes Lb2Cas12a effector proteins comprising one or more substitutions at certain residues with increased activity compared to wild-type Lb2Cas12a proteins. A similar strategy described by Example 1 was used to determine certain amino acid substitutions for improvement of activity(ies) of Lb2Cas12a effectors proteins described by this example.

Lb2Cas12a effector proteins are smaller (e.g., about 300 base pairs smaller) than other Cas12a orthologues (such as AsCas12a). Although, an Lb2Cas12a effector protein comprising Q571K and C1003Y substitutions increased activity compared to wild-type Lb2Cas12a (see Tran et al., Molecular Therapy Nucleic Acids, 24:P40-53 (2021), the disclosure of which is hereby incorporated herein by reference in its entirety), it is contemplated by the present disclosure that additional mutations made to Lb2Cas12a may further increase activity and make this Cas12a orthologue more attractive for genome editing applications.

As described in Example 1, an AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety). It is an insight of the present disclosure that Lb2Cas12a effector proteins and AsCas12a effector proteins share sequence conservation of domains (see FIG. 1A). Without wishing to be bound by any theory, the present example provides Lb2Cas12a effector proteins with increased activity that are achieved by mutating residues in these conserved regions. After mapping, it was found that wild-type Lb2Cas12a (SEQ ID NO: 3) also already contains an R amino acid at the corresponding position of M537R AsCas12a (see FIG. 1A). However, residue 778 of Lb2Cas12a is readily amenable to mutagenesis and can be mutated to T778L. Accordingly, the present example describes an Lb2Cas12a effector protein having an amino acid substitution of T778L relative to SEQ ID NO: 3, which is expected to result in a higher activity Lb2Cas12a relative to the wild-type protein.

Additional combinations of amino acid substitutions (mutations) relative to exemplary Lb2Cas12a wild-type amino acid sequence SEQ ID NO: 3 are shown in Table 10. Alignments of Lb2Cas12a sequences and exemplary substitutions as described herein are provided in FIGS. 2, 3, and 4A-B. In some cases, an Lb2Cas12a effector protein can comprise or consist of any of the combinations of amino acid substitutions present in Table 10 relative to an exemplary Lb2Cas12a wild-type effector protein (SEQ ID NO: 3).

TABLE 10
Lb2Cas12a Variants with substitutions relative to SEQ ID NO: 3

| | T778L | K155R | Q571K | C1003Y | S167K | E271R | K926R |
|---|---|---|---|---|---|---|---|
| 1 | X | | | | | | |
| 2 | | X | | | | | |
| 3 | | | X | | | | |
| 4 | | | | X | | | |
| 5 | X | X | | | | | |
| 6 | X | | X | | | | |
| 7 | X | | | X | | | |
| 8 | X | X | X | | | | |
| 9 | X | X | | X | | | |
| 10 | X | X | X | X | | | |
| 11 | | X | X | X | | | |
| 12 | | | | | X | | |
| 13 | | | | | | X | |
| 14 | | | | | | | X |
| 15 | | | | | X | X | |
| 16 | | | | | X | | X |
| 17 | | | | | | X | X |
| 18 | | | | | X | X | X |
| 19 | X | | | | X | | |
| 20 | X | | | | | X | |
| 21 | X | | | | | | X |
| 22 | X | | | | X | X | |
| 23 | X | | | | X | | X |
| 24 | X | | | | | X | X |
| 25 | X | | | | X | X | X |
| 26 | X | | | X | X | X | X |
| 27 | X | | X | X | X | X | X |
| 28 | X | X | | X | X | X | X |
| 29 | X | X | X | X | X | X | X |

### Example 6: Lb2Cas12a Effector Protein Variants (Nickases) with Increased Activity

The present example describes Lb2Cas12a effector proteins (nickases) comprising one or more substitutions at certain residues with increased activity compared to wild-type Lb2Cas12a proteins.

The disclosure also contemplates that certain amino acid residues of an Lb2Cas12a effector protein may be substituted (or mutated) to generate Lb2Cas12a effector proteins that are nickases, optionally with increased activity(ies). For example, the present disclosure describes that K913 and/or R914 residues of an amino acid sequence provided in SEQ ID NO: 3 can be substituted (or mutated).

An AsCas12a effector protein comprising two or more substitutions (e.g., K1000G and S1001G) has been demonstrated to result in a nickase version of wild-type AsCas12a (SEQ ID NO: 1). It is an insight of the present disclosure that Lb2Cas12a effector proteins and AsCas12a effector proteins share sequence conservation around these positions (see FIG. 5). Without wishing to be bound by any theory, the present example provides Lb2Cas12a effector proteins with nickase activity by mutating residues in these conserved regions. For example, without wishing to be bound by any theory, the present example describes that mutating residues K913 and/or R914 of Lb2Cas12a is expected to produce Lb2Cas12a effector proteins with nickase activity. In particular, mutations residues K913 and/or R914 of Lb2Cas12a to have K913G and/or R914G, which correspond to K100G and S1001G in an exemplary AsCas12a effector protein (SEQ ID NO: 1), is expected to result in an Lb2Cas12a effector protein with nickase activity.

An AsCas12a effector protein comprising a substitution (e.g., R1226A) has been demonstrated to result in a nickase version of an AsCas12a effector protein (SEQ ID NO: 83). It is an insight of the present disclosure that Lb2Cas12a effector proteins and AsCas12a effector proteins share sequence conservation around these positions (see FIG. 5). Without wishing to be bound by any theory, the present example provides Lb2Cas12a effector proteins with nickase activity by mutating residues in these conserved regions. For example, without wishing to be bound by any theory, the present example describes that mutating a residue R1124 of Lb2Cas12a is expected to produce Lb2Cas12a effector proteins with nickase activity. In particular, mutation of residue R1124 of Lb2Cas12a to have R1124A, which correspond to R1226A in an exemplary AsCas12a effector protein (SEQ ID NO: 1), is expected to result in an Lb2Cas12a effector protein with nickase activity.

Additional combinations of these nickase-inducing mutations with amino acid substitutions (mutations) that increase activity relative to exemplary Lb2Cas12a wild-type amino acid sequence SEQ ID NO: 3 are shown in Table 11. Alignments of Lb2Cas12a sequences and exemplary substitutions as described herein are provided in FIGS. 2, 3, and 4A-B. In some cases, an Lb2Cas12a effector protein can comprise or consist of any of the combinations of amino acid substitutions present in Table 11 relative to an exemplary Lb2Cas12a wild-type effector protein (SEQ ID NO: 3).

TABLE 11
Lb2Cas12a Variants with substitutions relative to SEQ ID NO: 3

| | T778L | K155R | Q571K | C1003Y | S167K | E271R | K1026R | K913G | R914G | R1124A |
|---|---|---|---|---|---|---|---|---|---|---|
| 1 | | | | | | | | X | | |
| 2 | | | | | | | | | X | |
| 3 | | | | | | | | X | X | |
| 4 | X | | | | | | | X | | |
| 5 | X | | | | | | | | X | |
| 6 | X | | | | | | | X | X | |
| 7 | | X | | | | | | X | | |
| 8 | | X | | | | | | | X | |
| 9 | | X | | | | | | X | X | |
| 10 | | | X | | | | | X | | |
| 11 | | | X | | | | | | X | |
| 12 | | | X | | | | | X | X | |
| 13 | | | | X | | | | X | | |
| 14 | | | | X | | | | | X | |
| 15 | | | | X | | | | X | X | |
| 16 | | | X | X | | | | X | | |
| 17 | | | X | X | | | | | X | |
| 18 | | | X | X | | | | X | X | |
| 19 | | X | | X | | | | X | | |
| 20 | | X | | X | | | | | X | |
| 21 | | X | | X | | | | X | X | |
| 22 | X | X | | | | | | X | | |
| 23 | X | X | | | | | | | X | |
| 24 | X | X | | | | | | X | X | |
| 25 | X | X | X | | | | | X | | |
| 26 | X | X | X | | | | | | X | |
| 27 | X | X | X | | | | | X | X | |
| 28 | X | | | X | | | | X | | |
| 29 | X | | | X | | | | | X | |
| 30 | X | | | X | | | | X | X | |
| 31 | X | X | | | | | | X | | |
| 32 | X | X | | X | | | | | X | |
| 33 | X | X | | X | | | | X | X | |
| 34 | X | X | X | X | | | | X | | |
| 35 | X | X | X | X | | | | | X | |
| 36 | X | X | X | X | | | | X | X | |
| 37 | | | | | X | | | X | | |
| 38 | | | | | X | | | | X | |
| 39 | | | | | X | | | X | X | |
| 40 | | | | | | X | | X | | |
| 41 | | | | | | X | | | X | |
| 42 | | | | | | X | | X | X | |
| 43 | | | | | | | X | X | | |
| 44 | | | | | | | X | | X | |
| 45 | | | | | | | X | X | X | |
| 46 | | | | | X | X | | X | | |
| 47 | | | | | X | X | | | X | |
| 48 | | | | | X | X | | X | X | |
| 49 | | | | | X | | X | X | | |
| 50 | | | | | X | | X | | X | |
| 51 | | | | | X | | X | X | X | |
| 52 | | | | | | X | X | X | | |
| 53 | | | | | | X | X | | X | |
| 54 | | | | | | X | X | X | X | |
| 55 | | | | | X | X | X | X | | |
| 56 | | | | | X | X | X | | X | |
| 57 | | | | | X | X | X | X | X | |
| 58 | X | | | | X | | | X | | |
| 59 | X | | | | X | | | | X | |
| 60 | X | | | | X | | | X | X | |
| 61 | X | | | | | X | | X | | |
| 62 | X | | | | | X | | | X | |
| 63 | X | | | | | X | | X | X | |
| 64 | X | | | | | | X | X | | |
| 65 | X | | | | | | X | | X | |
| 66 | X | | | | | | X | X | X | |
| 67 | X | | | | X | X | | X | | |
| 68 | X | | | | X | X | | | X | |
| 69 | X | | | | X | X | | X | X | |
| 70 | X | | | | X | | X | X | | |
| 71 | X | | | | X | | X | | X | |
| 72 | X | | | | X | | X | X | X | |
| 73 | X | | | | | X | X | X | | |
| 74 | X | | | | | X | X | | X | |
| 75 | X | | | | | X | X | X | X | |
| 76 | X | | | | X | X | X | X | | |
| 77 | X | | | | X | X | X | | X | |
| 78 | X | | | | X | X | X | X | X | |
| 79 | X | | | X | X | X | X | X | | |
| 80 | X | | | X | X | X | X | | X | |
| 81 | X | | | X | X | X | X | X | X | |
| 82 | X | | X | X | X | X | X | X | | |
| 83 | X | | X | X | X | X | X | | X | |
| 84 | X | | X | X | X | X | X | X | X | |
| 85 | X | X | X | X | X | X | X | X | | |
| 86 | X | X | X | X | X | X | X | | X | |
| 87 | X | X | X | X | X | X | X | X | X | |
| 88 | | | | | | | | | | X |
| 89 | X | | | | | | | | | X |
| 90 | | X | | | | | | | | X |
| 91 | | | X | | | | | | | X |
| 92 | | | | X | | | | | | X |
| 93 | | | X | X | | | | | | X |
| 94 | | X | | X | | | | | | X |
| 95 | X | X | | | | | | | | X |
| 96 | X | X | X | | | | | | | X |
| 97 | X | | | X | | | | | | X |
| 98 | X | X | | X | | | | | | X |
| 99 | X | X | X | X | | | | | | X |
| 100 | | | | | X | | | | | X |
| 101 | | | | | | X | | | | X |
| 102 | | | | | | | X | | | X |
| 103 | | | | | X | X | | | | X |
| 104 | | | | | X | | X | | | X |
| 105 | | | | | | X | X | | | X |
| 106 | | | | | X | X | X | | | X |
| 107 | X | | | | X | | | | | X |
| 108 | X | | | | | X | | | | X |
| 109 | X | | | | | | X | | | X |
| 110 | X | | | | X | X | | | | X |
| 111 | X | | | | X | | X | | | X |
| 112 | X | | | | | X | X | | | X |
| 113 | X | | | | X | X | X | | | X |
| 114 | X | | | X | X | X | X | | | X |
| 115 | X | | X | X | X | X | X | | | X |
| 116 | X | X | X | X | X | X | X | | | X |

In some embodiments, an Lb2Cas12a effector protein can comprise a combination of the R1218A mutation with mutations of residues E184, N607, and K613 of Lb2Cas12a to produce effector proteins with increased nickase activity. In particular, mutation of residues K155, N512, and K518 of FnCas12a to have K155R, N512R, and K518R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into Lb2Cas12a effector (nickase) proteins.

### Example 7: LbCas12a Effector Protein Variants (Nucleases) with Increased Activity

The present example describes LbCas12a effector proteins comprising one or more substitutions at certain residues with increased activity compared to wild-type LbCas12a proteins. A similar strategy described by Example 1 was used to determine certain amino acid substitutions for improvement of activity(ies) of LbCas12a effectors proteins described by this example.

As described in Example 1, an AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety). It is an insight of the present disclosure that LbCas12a effector proteins and AsCas12a effector proteins share sequence conservation of domains (see FIG. 1A). Without wishing to be bound by any theory, the present example provides LbCas12a effector proteins with increased activity that are achieved by mutating residues in these conserved regions. For example, without wishing to be bound by any theory, the present example describes that mutating residues N527 and/or E795 of LbCas12a is expected to produce LbCas12a effector proteins with higher activity compared to wild-type LbCas12a effector proteins. In particular, mutating residues N527 and/or E795 of LbCas12a to have N527R and/or E795L mutations, which correspond to M537R and F870L in an exemplary AsCas12a protein (SEQ ID NO: 1), is expected to result in a higher activity LbCas12a relative to the wild-type protein.

Additional combinations of amino acid substitutions (mutations) relative to exemplary LbCas12a wild-type amino acid sequence SEQ ID NO: 4 are shown in Table 12. Alignments of LbCas12a sequences and exemplary substitutions as described herein are provided in FIGS. 2, 3, and 4A-B. In some cases, an LbCas12a effector protein can comprise or consist of any of the combinations of amino acid substitutions present in Table 12 relative to an exemplary LbCas12a wild-type effector protein (SEQ ID NO: 4).

TABLE 12
LbCas12a Variants with substitutions relative to SEQ ID NO: 4

| | N527R | E795L | D156R | S168K | S286R | K945R |
|---|---|---|---|---|---|---|
| 1 | X | X | | | | |
| 2 | | | X | | | |
| 3 | X | | X | | | |
| 4 | | X | X | | | |
| 5 | X | X | X | | | |
| 6 | | | | X | | |
| 7 | | | | | X | |
| 8 | | | | | | X |
| 9 | | | | X | X | |
| 10 | | | | X | | X |
| 11 | | | | | X | X |
| 12 | | | | X | X | X |
| 13 | X | | | X | | |
| 14 | X | | | | X | |
| 15 | X | | | | | X |
| 16 | X | | | X | X | |
| 17 | X | | | X | | X |
| 18 | X | | | | X | X |
| 19 | X | | | X | X | X |
| 20 | X | | X | X | X | X |
| 21 | X | | X | X | X | X |
| 22 | | X | | X | | |
| 23 | | X | | | X | |
| 24 | | X | | | | X |
| 25 | | X | | X | X | |
| 26 | | X | | X | | X |
| 27 | | X | | | X | X |
| 28 | | X | | X | X | X |
| 29 | | X | X | X | X | X |
| 30 | | X | X | X | X | X |
| 31 | X | X | | X | | |
| 32 | X | X | | | X | |
| 33 | X | X | | | | X |
| 34 | X | X | | X | X | |
| 35 | X | X | | X | | X |
| 36 | X | X | | | X | X |
| 37 | X | X | | X | X | X |
| 38 | X | X | X | X | X | X |
| 39 | X | X | X | X | X | X |

### Example 8: LbCas12a Effector Protein Variants (Nickases) with Increased Activity

The present example describes LbCas12a effector proteins (nickases) comprising one or more substitutions at certain residues with increased activity compared to wild-type LbCas12a proteins.

The disclosure contemplates that certain amino acid residues of an LbCas12a effector protein may be substituted (or mutated) to generate LbCas12a effector proteins that are nickases, optionally with increased activity(ies). For example, the present disclosure describes that K932 and/or N933 residues of an amino acid sequence provided in SEQ ID NO: 4 can be substituted (or mutated).

An AsCas12a effector protein comprising two or more substitutions (e.g., K1000G and S1001G) has been demonstrated to result in a nickase version of wild-type AsCas12a (SEQ ID NO: 1). It is an insight of the present disclosure that LbCas12a effector proteins and AsCas12a effector proteins share sequence conservation around these positions (see FIG. 5). Without wishing to be bound by any theory, the present example provides LbCas12a effector proteins with nickase activity by mutating residues in these conserved regions. For example, without wishing to be bound by any theory, the present example describes that mutating residues K932 and/or N933 of LbCas12a is expected to produce LbCas12a effector proteins with nickase activity. In particular, mutations residues K932 and/or N933 of LbCas12a to have K932G and/or N933G, which correspond to K100G and S1001G in an exemplary AsCas12a effector protein (SEQ ID NO: 1), is expected to result in an LbCas12a effector protein with nickase activity.

An AsCas12a effector protein comprising a substitution (e.g., R1226A) has been demonstrated to result in a nickase version of an AsCas12a effector protein (SEQ ID NO: 83). It is an insight of the present disclosure that LbCas12a effector proteins and AsCas12a effector proteins share sequence conservation around these positions (see FIG. 5). Without wishing to be bound by any theory, the present example provides LbCas12a effector proteins with nickase activity by mutating residues in these conserved regions. For example, without wishing to be bound by any theory, the present example describes that mutating a residue R1138 of LbCas12a is expected to produce LbCas12a effector proteins with nickase activity. In particular, mutation of residue R1138 of LbCas12a to have R1138A, which correspond to R1226A in an exemplary AsCas12a effector protein (SEQ ID NO: 1), is expected to result in an LbCas12a effector protein with nickase activity.

Additional combinations of these nickase-inducing mutations with amino acid substitutions (mutations) that increase activity relative to exemplary LbCas12a wild-type amino acid sequence SEQ ID NO: 4 are shown in Table 13. Alignments of LbCas12a sequences and exemplary substitutions as described herein are provided in FIGS. 2, 3, and 4A-B. In some cases, an LbCas12a effector protein can comprise or consist of any of the combinations of amino acid substitutions present in Table 13 relative to an exemplary LbCas12a wild-type effector protein (SEQ ID NO: 4).

TABLE 13
LbCas12a Variants with substitutions relative to SEQ ID NO: 4

| | N527R | E795L | D156R | S168K | S286R | K945R | K932G | N933G | R1138A |
|---|---|---|---|---|---|---|---|---|---|
| 1 | | | | | | | X | | |
| 2 | | | | | | | | X | |
| 3 | | | | | | | X | X | |
| 4 | X | | | | | | X | | |
| 5 | X | | | | | | | X | |
| 6 | | X | | | | | X | | |
| 7 | | X | | | | | | X | |
| 8 | X | X | | | | | X | | |
| 9 | X | X | | | | | | X | |
| 10 | X | X | | | | | X | X | |
| 11 | | | X | | | | X | | |
| 12 | | | X | | | | | X | |
| 13 | | | X | | | | X | X | |
| 14 | X | X | X | | | | X | | |
| 15 | X | X | X | | | | | X | |
| 16 | X | X | X | | | | X | X | |
| 17 | | | | X | | | X | | |
| 18 | | | | X | | | | X | |
| 19 | | | | X | | | X | X | |
| 20 | | | | | X | | X | | |
| 21 | | | | | X | | | X | |
| 22 | | | | | X | | X | X | |
| 23 | | | | | | X | X | | |
| 24 | | | | | | X | | X | |
| 25 | | | | | | X | X | X | |
| 26 | | | | X | X | | X | | |
| 27 | | | | X | X | | | X | |
| 28 | | | | X | X | | X | X | |
| 29 | | | | X | | X | X | | |
| 30 | | | | X | | X | | X | |
| 31 | | | | X | | X | X | X | |
| 32 | | | | | X | X | X | | |
| 33 | | | | | X | X | | X | |
| 34 | | | | | X | X | X | X | |
| 35 | | | | X | X | X | X | | |
| 36 | | | | X | X | X | | X | |
| 37 | | | | X | X | X | X | X | |
| 38 | X | | | X | | | X | | |
| 39 | X | | | X | | | | X | |
| 40 | X | | | X | | | X | X | |
| 41 | X | | | | X | | X | | |
| 42 | X | | | | X | | | X | |
| 43 | X | | | | X | | X | X | |
| 44 | X | | | | | X | X | | |
| 45 | X | | | | | X | | X | |
| 46 | X | | | | | X | X | X | |
| 47 | X | | | X | X | | X | | |
| 48 | X | | | X | X | | | X | |
| 49 | X | | | X | X | | X | X | |
| 50 | X | | | X | | X | X | | |
| 51 | X | | | X | | X | | X | |
| 52 | X | | | X | | X | X | X | |
| 53 | X | | | | X | X | X | | |
| 54 | X | | | | X | X | | X | |
| 55 | X | | | | X | X | X | X | |
| 56 | X | | | X | X | X | X | | |
| 57 | X | | | X | X | X | | X | |
| 58 | X | | | X | X | X | X | X | |
| 59 | X | | X | X | X | X | X | X | |
| 60 | | X | | X | | | X | | |
| 61 | | X | | X | | | | X | |
| 62 | | X | | X | | | X | X | |
| 63 | | X | | | X | | X | | |
| 64 | | X | | | X | | | X | |
| 65 | | X | | | X | | X | X | |
| 66 | | X | | | | X | X | | |
| 67 | | X | | | | X | | X | |
| 68 | | X | | | | X | X | X | |
| 69 | | X | | X | X | | X | | |
| 70 | | X | | X | X | | | X | |
| 71 | | X | | X | X | | X | X | |
| 72 | | X | | X | | X | X | | |
| 73 | | X | | X | | X | | X | |
| 74 | | X | | X | | X | X | X | |
| 75 | | X | | | X | X | X | | |
| 76 | | X | | | X | X | | X | |
| 77 | | X | | | X | X | X | X | |
| 78 | | X | | X | X | X | X | | |
| 79 | | X | | X | X | X | | X | |
| 80 | | X | | X | X | X | X | X | |
| 81 | | X | X | X | X | X | X | X | |
| 82 | X | X | | X | | | X | | |
| 83 | X | X | | X | | | | X | |
| 84 | X | X | | X | | | X | X | |
| 85 | X | X | | | X | | X | | |
| 86 | X | X | | | X | | | X | |
| 87 | X | X | | | X | | X | X | |
| 88 | X | X | | | | X | X | | |
| 89 | X | X | | | | X | | X | |
| 90 | X | X | | | | X | X | X | |
| 91 | X | X | | X | X | | X | | |
| 92 | X | X | | X | X | | | X | |
| 93 | X | X | | X | X | | X | X | |
| 94 | X | X | | X | | X | X | | |
| 95 | X | X | | X | | X | | X | |
| 96 | X | X | | X | | X | X | X | |
| 97 | X | X | | | X | X | X | | |
| 98 | X | X | | | X | X | | X | |
| 99 | X | X | | | X | X | X | X | |
| 100 | X | X | | X | X | X | X | | |
| 101 | X | X | | X | X | X | | X | |
| 102 | X | X | | X | X | X | X | X | |
| 103 | X | X | X | X | X | X | X | X | |
| 104 | | | | | | | | | X |
| 105 | X | | | | | | | | X |
| 106 | | X | | | | | | | X |
| 107 | X | X | | | | | | | X |
| 108 | | | X | | | | | | X |
| 109 | X | X | X | | | | | | X |
| 110 | | | | X | | | | | X |
| 111 | | | | | X | | | | X |
| 112 | | | | | | X | | | X |
| 113 | | | | X | X | | | | X |
| 114 | | | | X | | X | | | X |
| 115 | | | | | X | X | | | X |
| 116 | | | | X | X | X | | | X |
| 117 | X | | | X | | | | | X |
| 118 | X | | | | X | | | | X |
| 119 | X | | | | | X | | | X |
| 120 | X | | | X | X | | | | X |
| 121 | X | | | X | | X | | | X |
| 122 | X | | | X | | X | | | X |
| 123 | X | | | X | | X | | | X |
| 124 | X | | | | X | X | | | X |
| 125 | X | | | X | X | X | | | X |
| 126 | X | | X | X | X | X | | | X |
| 127 | | X | | X | | | | | X |
| 128 | | X | | | X | | | | X |
| 129 | | X | | | | X | | | X |
| 130 | | X | | X | X | | | | X |
| 131 | | X | | X | | X | | | X |
| 132 | | X | | | X | X | | | X |
| 133 | | X | | X | X | X | | | X |
| 134 | | X | X | X | X | X | | | X |
| 135 | X | X | | X | | | | | X |
| 136 | X | X | | | X | | | | X |
| 137 | X | X | | | | X | | | X |
| 138 | X | X | | X | X | | | | X |
| 139 | X | X | | X | | X | | | X |
| 140 | X | X | | | X | X | | | X |
| 141 | X | X | | X | X | X | | | X |
| 142 | X | X | X | X | X | X | | | X |

In some embodiments, an LbCas12a effector protein can comprise a combination of the R1138 mutation with mutations of residues D156, G532, and K538 of LbCas12a to produce effector proteins with increased nickase activity. In particular, mutation of residues D156, G532, and K538 of LbCas12a to have D156R, G532R, and K538R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into LbCas12a effector (nickase) proteins.

### Example 9: MbCas12a Effector Protein Variants (Nucleases) with Increased Activity

The present example describes MbCas12a effector proteins comprising one or more mutations at certain residues with increased activity compared to wild-type MbCas12a proteins. A similar strategy described by Example 1 was used to determine certain amino acid substitutions for improvement of activity(ies) of MbCas12a effectors proteins described by this example.

As described in Example 1, an AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety). It is an insight of the present disclosure that MbCas12a effector proteins and AsCas12a effector proteins share sequence conservation of domains (see FIG. 1A). Without wishing to be bound by any theory, the present example provides MbCas12a effector proteins with increased activity that are achieved by mutating residues in these conserved regions. For example, without wishing to be bound by any theory, the present example describes that mutating residues N568 and/or M825 of MbCas12a is expected to produce MbCas12a effector proteins with higher activity compared to wild-type MbCas12a effector proteins. In particular, mutating residues N568 and/or M825 of MbCas12a to have N568R and/or M825L mutations, which correspond to M537R and F870L in an exemplary AsCas12a protein (SEQ ID NO: 1), is expected to result in a higher activity MbCas12a relative to the wild-type protein.

Additional combinations of amino acid substitutions (mutations) relative to exemplary MbCas12a wild-type amino acid sequence SEQ ID NO: 5 are shown in Table 14. Alignments of MbCas12a sequences and exemplary substitutions as described herein are provided in FIGS. 2, 3, and 4A-B. In some cases, an MbCas12a effector protein can comprise or consist of any of the combinations of amino acid substitutions present in Table 14 relative to an exemplary MbCas12a wild-type effector protein (SEQ ID NO: 5).

TABLE 14
MbCas12a Variants with substitutions relative to SEQ ID NO: 5

| | N568R | M825L | D172R | H184K | G292R | N978R |
|---|---|---|---|---|---|---|
| 1 | X | X | | | | |
| 2 | | | X | | | |
| 3 | X | | X | | | |
| 4 | | X | | | | |
| 5 | X | X | X | | | |
| 6 | | | | X | | |
| 7 | | | | | X | |
| 8 | | | | | | X |
| 9 | | | | X | X | |
| 10 | | | | X | | X |
| 11 | | | | | X | X |
| 12 | | | | X | X | X |
| 13 | X | | | X | | |
| 14 | X | | | | X | |
| 15 | X | | | | | X |
| 16 | X | | | X | X | |
| 17 | X | | | X | | X |
| 18 | X | | | | X | X |
| 19 | X | | | X | X | X |
| 20 | X | | X | X | X | X |
| 21 | X | | X | X | X | X |
| 22 | | X | | X | | |
| 23 | | X | | | X | |
| 24 | | X | | | | X |
| 25 | | X | | X | X | |
| 26 | | X | | X | | X |
| 27 | | X | | | X | X |
| 28 | | X | | X | X | X |
| 29 | | X | X | X | X | X |
| 30 | | X | X | X | X | X |
| 31 | X | X | | X | | |
| 32 | X | X | | | X | |
| 33 | X | X | | | | X |
| 34 | X | X | | X | X | |
| 35 | X | X | | X | | X |
| 36 | X | X | | | X | X |
| 37 | X | X | | X | X | X |
| 38 | X | X | X | X | X | X |
| 39 | X | X | X | X | X | X |

### Example 10: MbCas12a Effector Protein Variants (Nickases) with Increased Activity

The present example describes MbCas12a effector proteins (nickases) comprising one or more substitutions at certain residues with increased activity compared to wild-type MbCas12a proteins.

The disclosure contemplates that certain amino acid residues of an MbCas12a effector protein may be substituted (or mutated) to generate MbCas12a effector proteins that are nickases, optionally with increased activity(ies). For example, the present disclosure describes that K965 and/or R966 residues of an amino acid sequence provided in SEQ ID NO: 5 can be substituted (or mutated).

An AsCas12a effector protein comprising two or more substitutions (e.g., K1000G and S1001G) has been demonstrated to result in a nickase version of wild-type AsCas12a (SEQ ID NO: 1). It is an insight of the present disclosure that MbCas12a effector proteins and AsCas12a effector proteins share sequence conservation around these positions (see FIG. 5). Without wishing to be bound by any theory, the present example provides MbCas12a effector proteins with nickase activity by mutating residues in these conserved regions. For example, without wishing to be bound by any theory, the present example describes that mutating residues K965 and/or R966 of MbCas12a is expected to produce MbCas12a effector proteins with nickase activity. In particular, mutations residues K965 and/or R966 of MbCas12a to have K965G and/or R966G, which correspond to K100G and S1001G in an exemplary AsCas12a effector protein (SEQ ID NO: 1), is expected to result in an MbCas12a effector protein with nickase activity.

An AsCas12a effector protein comprising a substitution (e.g., R1226A) has been demonstrated to result in a nickase version of an AsCas12a effector protein (SEQ ID NO: 83). It is an insight of the present disclosure that MbCas12a effector proteins and AsCas12a effector proteins share sequence conservation around these positions (see FIG. 5). Without wishing to be bound by any theory, the present example provides MbCas12a effector proteins with nickase activity by mutating residues in these conserved regions. For example, without wishing to be bound by any theory, the present example describes that mutating a residue R1171 of MbCas12a is expected to produce MbCas12a effector proteins with nickase activity. In particular, mutation of residue R1171 of MbCas12a to have R1171A, which correspond to R1226A in an exemplary AsCas12a effector protein (SEQ ID NO: 1), is expected to result in an MbCas12a effector protein with nickase activity.

Additional combinations of these nickase-inducing mutations with amino acid substitutions (mutations) that increase activity relative to exemplary MbCas12a wild-type amino acid sequence SEQ ID NO: 5 are shown in Table 15. Alignments of MbCas12a sequences and exemplary substitutions as described herein are provided in FIGS. 2, 3, and 4A-B. In some cases, an MbCas12a effector protein can comprise or consist of any of the combinations of amino acid substitutions present in Table 15 relative to an exemplary MbCas12a wild-type effector protein (SEQ ID NO: 5).

TABLE 15
MbCas12a Variants with substitutions relative to SEQ ID NO: 5

| | N568R | M825L | D172R | H184K | G292R | N978R | K965G | R966G | R1171A |
|---|---|---|---|---|---|---|---|---|---|
| 1 | | | | | | | X | | |
| 2 | | | | | | | | X | |
| 3 | | | | | | | X | X | |
| 4 | X | | | | | | X | | |
| 5 | X | | | | | | | X | |
| 6 | | X | | | | | X | | |
| 7 | | X | | | | | | X | |
| 8 | X | X | | | | | X | | |
| 9 | X | X | | | | | | X | |
| 10 | X | X | | | | | X | X | |
| 11 | | | X | | | | X | | |
| 12 | | | X | | | | | X | |
| 13 | | | X | | | | X | X | |
| 14 | X | X | X | | | | X | | |
| 15 | X | X | X | | | | | X | |
| 16 | X | X | X | | | | X | X | |
| 17 | | | | X | | | X | | |
| 18 | | | | X | | | | X | |
| 19 | | | | X | | | X | X | |
| 20 | | | | | X | | X | | |
| 21 | | | | | X | | | X | |
| 22 | | | | | X | | X | X | |
| 23 | | | | | | X | X | | |
| 24 | | | | | | X | | X | |
| 25 | | | | | | X | X | X | |
| 26 | | | | X | X | | X | | |
| 27 | | | | X | X | | | X | |
| 28 | | | | X | X | | X | X | |
| 29 | | | | X | | X | X | | |
| 30 | | | | X | | X | | X | |
| 31 | | | | X | | X | X | X | |
| 32 | | | | | X | X | X | | |
| 33 | | | | | X | X | | X | |
| 34 | | | | | X | X | X | X | |
| 35 | | | | X | X | X | X | | |
| 36 | | | | X | X | X | | X | |
| 37 | | | | X | X | X | X | X | |
| 38 | X | | | X | | | X | | |
| 39 | X | | | X | | | | X | |
| 40 | X | | | X | | | X | X | |
| 41 | X | | | | X | | X | | |
| 42 | X | | | | X | | | X | |
| 43 | X | | | | X | | X | X | |
| 44 | X | | | | | X | X | | |
| 45 | X | | | | | X | | X | |
| 46 | X | | | | | X | X | X | |
| 47 | X | | | X | X | | X | | |
| 48 | X | | | X | X | | | X | |
| 49 | X | | | X | X | | X | X | |
| 50 | X | | | X | | X | X | | |
| 51 | X | | | X | | X | | X | |
| 52 | X | | | X | | X | X | X | |
| 53 | X | | | | X | X | X | | |
| 54 | X | | | | X | X | | X | |
| 55 | X | | | | X | X | X | X | |
| 56 | X | | | X | X | X | X | | |
| 57 | X | | | X | X | X | | X | |
| 58 | X | | | X | X | X | X | X | |
| 59 | X | | X | X | X | X | X | X | |
| 60 | | X | | X | | | X | | |
| 61 | | X | | X | | | | X | |
| 62 | | X | | X | | | X | X | |
| 63 | | X | | | X | | X | | |
| 64 | | X | | | X | | | X | |
| 65 | | X | | | X | | X | X | |
| 66 | | X | | | | X | X | | |
| 67 | | X | | | | X | | X | |
| 68 | | X | | | | X | X | X | |
| 69 | | X | | X | X | | X | | |
| 70 | | X | | X | X | | | X | |
| 71 | | X | | X | X | | X | X | |
| 72 | | X | | X | | X | X | | |
| 73 | | X | | X | | X | | X | |
| 74 | | X | | X | | X | X | X | |
| 75 | | X | | | X | X | X | | |
| 76 | | X | | | X | X | | X | |
| 77 | | X | | | X | X | X | X | |
| 78 | | X | | X | X | X | X | | |
| 79 | | X | | X | X | X | | X | |
| 80 | | X | | X | X | X | X | X | |
| 81 | | X | X | X | X | X | X | X | |
| 82 | X | X | | X | | | X | | |
| 83 | X | X | | X | | | | X | |
| 84 | X | X | | X | | | X | X | |
| 85 | X | X | | | X | | X | | |
| 86 | X | X | | | X | | | X | |
| 87 | X | X | | | X | | X | X | |
| 88 | X | X | | | | X | X | | |
| 89 | X | X | | | | X | | X | |
| 90 | X | X | | | | X | X | X | |
| 91 | X | X | | X | X | | X | | |
| 92 | X | X | | X | X | | | X | |
| 93 | X | X | | X | X | | X | X | |
| 94 | X | X | | X | | X | X | | |
| 95 | X | X | | X | | X | | X | |
| 96 | X | X | | X | | X | X | X | |
| 97 | X | X | | | X | X | X | | |
| 98 | X | X | | | X | X | | X | |
| 99 | X | X | | | X | X | X | X | |
| 100 | X | X | | X | X | X | X | | |
| 101 | X | X | | X | X | X | | X | |
| 102 | X | X | | X | X | X | X | X | |
| 103 | X | X | X | X | X | X | X | X | |
| 104 | | | | | | | | | X |
| 105 | X | | | | | | | | X |
| 106 | | X | | | | | | | X |
| 107 | X | X | | | | | | | X |
| 108 | | | X | | | | | | X |
| 109 | X | X | X | | | | | | X |
| 110 | | | | X | | | | | X |
| 111 | | | | | X | | | | X |
| 112 | | | | | | X | | | X |
| 113 | | | | X | X | | | | X |
| 114 | | | | X | | X | | | X |
| 115 | | | | | X | X | | | X |
| 116 | | | | X | X | X | | | X |
| 117 | X | | | X | | | | | X |
| 118 | X | | | | X | | | | X |
| 119 | X | | | | | X | | | X |
| 120 | X | | | X | X | | | | X |
| 121 | X | | | X | | X | | | X |
| 122 | X | | | | X | X | | | X |
| 123 | X | | | X | X | X | | | X |
| 124 | X | | X | X | X | X | | | X |
| 125 | | X | | X | | | | | X |
| 126 | | X | | | X | | | | X |
| 127 | | X | | | | X | | | X |
| 128 | | X | | X | X | | | | X |
| 129 | | X | | X | | X | | | X |
| 130 | | X | | | X | X | | | X |
| 131 | | X | | X | X | X | | | X |
| 132 | | X | X | X | X | X | | | X |
| 133 | X | X | | X | | | | | X |
| 134 | X | X | | | X | | | | X |
| 135 | X | X | | | | X | | | X |
| 136 | X | X | | X | X | | | | X |
| 137 | X | X | | X | | X | | | X |
| 138 | X | X | | | X | X | | | X |
| 139 | X | X | | X | X | X | | | X |
| 140 | X | X | X | X | X | X | | | X |

In some embodiments, an MbCas12a effector protein can comprise a combination of the R1171 mutation with mutations of residues D172, N563, and K569 of MbCas12a to produce effector proteins with increased nickase activity. In particular, mutation of residues D172, N563, and K569 of MbCas12a to have D172R, N563R, and K569R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into MbCas12a effector (nickase) proteins.

### Example 11: MG29-1 Effector Protein Variants (Nucleases) with Increased Activity

The present example describes MG29-1 effector proteins comprising one or more mutations at certain residues with increased activity compared to naturally occurring MG29-1 proteins. A similar strategy described by Example 1 was used to determine certain amino acid substitutions for improvement of activity(ies) of MG29-1 effectors proteins described by this example.

As described in Example 1, an AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)), the disclosure of which is hereby incorporated herein by reference in its entirety). It is an insight of the present disclosure that MG29-1 effector proteins and AsCas12a effector proteins share sequence conservation of domains (see FIG. 1B). Without wishing to be bound by any theory, the present example provides MG29-1 effector proteins with increased activity that are achieved by mutating residues in these conserved regions. For example, without wishing to be bound by any theory, the present example describes that mutating residues A572 and/or F849 of MG29-1 is expected to produce MG29-1 effector proteins with higher activity compared to naturally occurring MG29-1 effector proteins. In particular, mutating residues A572 and/or F849 of MG29-1 to have A572R and/or F849L mutations, which correspond to M537R and F870L in an exemplary AsCas12a protein (SEQ ID NO: 1), is expected to result in a higher activity MG29-1 relative to the naturally occurring protein.

Additional combinations of amino acid substitutions (mutations) relative to exemplary naturally occurring MG29-1 amino acid sequence SEQ ID NO: 14 are shown in Table 16. Alignments of MG29-1 sequences and exemplary substitutions as described herein are provided in FIGS. 2, 3, and 4A-B. In some cases, an MG29-1 effector protein can comprise or consist of any of the combinations of amino acid substitutions present in Table 16 relative to an exemplary naturally occurring MG29-1 effector protein (SEQ ID NO: 14).

TABLE 16
MG29-1 Variants with substitutions relative to SEQ ID NO: 14

| | A572R | F849L | E172R | S184K | R292K | T306R | K996R | S168R |
|---|---|---|---|---|---|---|---|---|
| 1 | X | | | | | | | |
| 2 | | X | | | | | | |
| 3 | X | X | | | | | | |
| 4 | X | | X | | | | | |
| 5 | X | | | X | | | | |
| 6 | X | | | | X | | | |
| 7 | X | | | | | X | | |
| 8 | X | | | | | | X | |
| 9 | X | | X | X | | | | |
| 10 | X | | X | | X | | | |
| 11 | X | | X | | | X | | |
| 12 | X | | X | | | | X | |
| 13 | X | | | X | X | | | |
| 14 | X | | | X | | X | | |
| 15 | X | | | X | | | X | |
| 16 | X | | | | X | X | | |
| 17 | X | | | | X | | X | |
| 18 | X | | | | | X | X | |
| 19 | X | | X | X | X | | | |
| 20 | X | | X | X | | X | | |
| 21 | X | | X | X | | | X | |
| 22 | X | | X | | X | X | | |
| 23 | X | | X | | X | | X | |
| 24 | X | | X | | | X | X | |
| 25 | X | | | X | X | X | | |
| 26 | X | | | X | X | | X | |
| 27 | X | | | | X | X | X | |
| 28 | X | | X | X | X | X | | |
| 29 | X | | X | X | X | | X | |
| 30 | X | | X | | X | X | X | |
| 31 | X | | X | X | | X | X | |
| 32 | X | | X | | X | X | X | |
| 33 | | | | X | X | X | X | |
| 34 | X | | X | X | X | X | X | |
| 35 | X | | X | | | | | X |
| 36 | X | | | X | | | | X |
| 37 | X | | | | X | | | X |
| 38 | X | | | | | X | | X |
| 39 | X | | | | | | X | X |
| 40 | X | | X | X | | | | X |
| 41 | X | | X | | X | | | X |
| 42 | X | | X | | | X | | X |
| 43 | X | | X | | | | X | X |
| 44 | X | | | X | X | | | X |
| 45 | X | | | X | | X | | X |
| 46 | X | | | X | | | X | X |
| 47 | X | | | | X | X | | X |
| 48 | X | | | | X | | X | X |
| 49 | X | | | | | X | X | X |
| 50 | X | | X | X | X | | | X |
| 51 | X | | X | X | | X | | X |
| 52 | X | | X | X | | | X | X |
| 53 | X | | X | | X | X | | X |
| 54 | X | | X | | X | | X | X |
| 55 | X | | X | | | X | X | X |
| 56 | X | | | X | X | X | | X |
| 57 | X | | | X | X | | X | X |
| 58 | X | | | | X | X | X | X |
| 59 | X | | X | X | X | X | | X |
| 60 | X | | X | X | X | | X | X |
| 61 | X | | X | | X | X | X | X |
| 62 | X | | X | X | | X | X | X |
| 63 | X | | X | | X | X | X | X |
| 64 | | | | X | X | X | X | X |
| 65 | X | | X | X | X | X | X | X |
| 66 | | X | X | | | | | |
| 67 | | X | | X | | | | |
| 68 | | X | | | X | | | |
| 69 | | X | | | | X | | |
| 70 | | X | | | | | X | |
| 71 | | X | X | X | | | | |
| 72 | | X | X | | X | | | |
| 73 | | X | X | | | X | | |
| 74 | | X | X | | | | X | |
| 75 | | X | | X | X | | | |
| 76 | | X | | X | | X | | |
| 77 | | X | | X | | | X | |
| 78 | | X | | | X | X | | |
| 79 | | X | | | X | | X | |
| 80 | | X | | | | X | X | |
| 81 | | X | X | X | X | | | |
| 82 | | X | X | X | | X | | |
| 83 | | X | X | X | | | X | |
| 84 | | X | X | | X | X | | |
| 85 | | X | X | | X | | X | |
| 86 | | X | X | | | X | X | |
| 87 | | X | | X | X | X | | |
| 88 | | X | | X | X | | X | |
| 89 | | X | | | X | X | X | |
| 90 | | X | X | X | X | X | | |
| 91 | | X | X | X | X | | X | |
| 92 | | X | X | | X | X | X | |
| 93 | | X | X | X | | X | X | |
| 94 | | X | X | | X | X | X | |
| 95 | | X | | X | X | X | X | |
| 96 | | X | X | X | X | X | X | |
| 97 | | X | X | | | | | X |
| 98 | | X | | X | | | | X |
| 99 | | X | | | X | | | X |
| 100 | | X | | | | X | | X |
| 101 | | X | | | | | X | X |
| 102 | | X | X | X | | | | X |
| 103 | | X | X | | X | | | X |
| 104 | | X | X | | | X | | X |
| 105 | | X | X | | | | X | X |
| 106 | | X | | X | X | | | X |
| 107 | | X | | X | | X | | X |
| 108 | | X | | X | | | X | X |
| 109 | | X | | | X | X | | X |
| 110 | | X | | | X | | X | X |
| 111 | | X | | | | X | X | X |
| 112 | | X | X | X | X | | | X |
| 113 | | X | X | X | | X | | X |
| 114 | | X | X | X | | | X | X |
| 115 | | X | X | | X | X | | X |
| 116 | | X | X | | X | | X | X |
| 117 | | X | X | | | X | X | X |
| 118 | | X | | X | X | X | | X |
| 119 | | X | | X | X | | X | X |
| 120 | | X | | | X | X | X | X |
| 121 | | X | X | X | X | X | | X |
| 122 | | X | X | X | X | | X | X |
| 123 | | X | X | | X | X | X | X |
| 124 | | X | X | X | | X | X | X |
| 125 | | X | X | | X | X | X | X |
| 126 | | X | | X | X | X | X | X |
| 127 | | X | X | X | X | X | X | X |
| 128 | X | X | X | | | | | |
| 129 | X | X | | X | | | | |
| 130 | X | X | | | X | | | |
| 131 | X | X | | | | X | | |
| 132 | X | X | | | | | X | |
| 133 | X | X | X | X | | | | |
| 134 | X | X | X | | X | | | |
| 135 | X | X | X | | | X | | |
| 136 | X | X | X | | | | X | |
| 137 | X | X | | X | X | | | |
| 138 | X | X | | X | | X | | |
| 139 | X | X | | X | | | X | |
| 140 | X | X | | | X | X | | |
| 141 | X | X | | | X | | X | |
| 142 | X | X | | | | X | X | |
| 143 | X | X | X | X | X | | | |
| 144 | X | X | X | X | | X | | |
| 145 | X | X | X | X | | | X | |
| 146 | X | X | X | | X | X | | |
| 147 | X | X | X | | X | | X | |
| 148 | X | X | X | | | X | X | |
| 149 | X | X | | X | X | X | | |
| 150 | X | X | | X | X | | X | |
| 151 | X | X | | | X | X | X | |
| 152 | X | X | X | X | X | X | | |
| 153 | X | X | X | X | X | | X | |
| 154 | X | X | X | | X | X | X | |
| 155 | X | X | X | X | | X | X | |
| 156 | X | X | X | | X | X | X | |
| 157 | X | X | | X | X | X | X | |
| 158 | X | X | X | X | X | X | X | |
| 159 | X | X | X | | | | | X |
| 160 | X | X | | X | | | | X |
| 161 | X | X | | | X | | | X |
| 162 | X | X | | | | X | | X |
| 163 | X | X | | | | | X | X |
| 164 | X | X | X | X | | | | X |
| 165 | X | X | X | | X | | | X |
| 166 | X | X | X | | | X | | X |
| 167 | X | X | X | | | | X | X |
| 168 | X | X | | X | X | | | X |
| 169 | X | X | | X | | X | | X |
| 170 | X | X | | X | | | X | X |
| 171 | X | X | | | X | X | | X |
| 172 | X | X | | | X | | X | X |
| 173 | X | X | | | | X | X | X |
| 174 | X | X | X | X | X | | | X |
| 175 | X | X | X | X | | X | | X |
| 176 | X | X | X | X | | | X | X |
| 177 | X | X | X | | X | X | | X |
| 178 | X | X | X | | X | | X | X |
| 179 | X | X | X | | | X | X | X |
| 180 | X | X | | X | X | X | | X |
| 181 | X | X | | X | X | | X | X |
| 182 | X | X | | | X | X | X | X |
| 183 | X | X | X | X | X | X | | X |
| 184 | X | X | X | X | X | | X | X |
| 185 | X | X | X | | X | X | X | X |
| 186 | X | X | X | X | | X | X | X |
| 187 | X | X | X | | X | X | X | X |
| 188 | X | X | | X | X | X | X | X |
| 189 | X | X | X | X | X | X | X | X |

### Example 12: MG29-1 Effector Protein Variants (Nickases) with Increased Activity

The present example describes MG29-1 effector proteins (nickases) comprising one or more substitutions at certain residues with increased activity compared to naturally occurring MG29-1 proteins.

The disclosure contemplates that certain amino acid residues of an MG29-1 effector protein may be substituted (or mutated) to generate MG29-1 effector proteins that are nickases, optionally with increased activity(ies). For example, the present disclosure describes that K983 and/or R984 residues of an amino acid sequence provided in SEQ ID NO: 14 can be substituted (or mutated).

An AsCas12a effector protein comprising two or more substitutions (e.g., K1000G and S1001G) has been demonstrated to result in a nickase version of wild-type AsCas12a (SEQ ID NO: 1). It is an insight of the present disclosure that MG29-1 effector proteins and AsCas12a effector proteins share sequence conservation around these positions (see FIG. 5). Without wishing to be bound by any theory, the present example provides MG29-1 effector proteins with nickase activity by mutating residues in these conserved regions. For example, without wishing to be bound by any theory, the present example describes that mutating residues K983 and/or R984 of MG29-1 is expected to produce MG29-1 effector proteins with nickase activity. In particular, mutations residues K983 and/or R984 of MG29-1 to have K983G and/or R984G, which correspond to K100G and S1001G in an exemplary AsCas12a effector protein (SEQ ID NO: 1), is expected to result in an MG29-1 effector protein with nickase activity.

An AsCas12a effector protein comprising the R1226A mutation has been demonstrated to result in a nickase version of an AsCas12a effector protein (SEQ ID NO: 83). It is an insight of the present disclosure that MG29-1 effector proteins and AsCas12a effector proteins share sequence conservation around these positions (see FIG. 5). Without wishing to be bound by any theory, the present example provides MG29-1 effector proteins with nickase activity by mutating residues in these conserved regions. For example, without wishing to be bound by any theory, the present example describes that mutating a residue R1192 of MG29-1 is expected to produce MG29-1 effector proteins with nickase activity. In particular, mutation of residue R1192 of MG29-1 to have R1192A, which correspond to R1226A in an exemplary AsCas12a effector protein (SEQ ID NO: 1), is expected to result in an MG29-1 effector protein with nickase activity.

Additional combinations of these nickase-inducing mutations with amino acid substitutions (mutations) that increase activity relative to exemplary naturally occurring MG29-1 amino acid sequence SEQ ID NO: 14 are shown in Table 17. Alignments of MG29-1 sequences and exemplary substitutions as described herein are provided in FIGS. 2, 3, and 4A-B. In some cases, an MG29-1 effector protein can comprise or consist of any of the combinations of amino acid substitutions present in Table 17 relative to an exemplary naturally occurring MG29-1 effector protein (SEQ ID NO: 14).

TABLE 17
MG29-1 Variants with substitutions relative to SEQ ID NO: 14

| | A572R | F849L | E172R | S184K | R292K | T306R | K996R | S168R | K983G | R984G | R1192A |
|---|---|---|---|---|---|---|---|---|---|---|---|
| 1 | X | | | | | | | | X | | |
| 2 | | X | | | | | | | X | | |
| 3 | X | X | | | | | | | X | | |
| 4 | X | | X | | | | | | X | | |
| 5 | X | | | X | | | | | X | | |
| 6 | X | | | | X | | | | X | | |
| 7 | X | | | | | X | | | X | | |
| 8 | X | | | | | | X | | X | | |
| 9 | X | | X | X | | | | | X | | |
| 10 | X | | X | | X | | | | X | | |
| 11 | X | | X | | | X | | | X | | |
| 12 | X | | X | | | | X | | X | | |
| 13 | X | | | X | X | | | | X | | |
| 14 | X | | | X | | X | | | X | | |
| 15 | X | | | X | | | X | | X | | |
| 16 | X | | | | X | X | | | X | | |
| 17 | X | | | | X | | X | | X | | |
| 18 | X | | | | | X | X | | X | | |
| 19 | X | | X | X | X | | | | X | | |
| 20 | X | | X | X | | X | | | X | | |
| 21 | X | | X | X | | | X | | X | | |
| 22 | X | | X | | X | X | | | X | | |
| 23 | X | | X | | X | | X | | X | | |
| 24 | X | | X | | | X | X | | X | | |
| 25 | X | | | X | X | X | | | X | | |
| 26 | X | | | X | X | | X | | X | | |
| 27 | X | | | | X | X | X | | X | | |
| 28 | X | | X | X | X | X | | | X | | |
| 29 | X | | X | X | X | | X | | X | | |
| 30 | X | | X | | X | X | X | | X | | |
| 31 | X | | X | X | | X | X | | X | | |
| 32 | X | | X | | X | X | X | | X | | |
| 33 | | | | X | X | X | X | | X | | |
| 34 | X | | X | X | X | X | X | | X | | |
| 35 | X | | X | | | | | X | X | | |
| 36 | X | | | X | | | | X | X | | |
| 37 | X | | | | X | | | X | X | | |
| 38 | X | | | | | X | | X | X | | |
| 39 | X | | | | | | X | X | X | | |
| 40 | X | | X | X | | | | X | X | | |
| 41 | X | | X | | X | | | X | X | | |
| 42 | X | | X | | | X | | X | X | | |
| 43 | X | | X | | | | X | X | X | | |
| 44 | X | | | X | X | | | X | X | | |
| 45 | X | | | X | | X | | X | X | | |
| 46 | X | | | X | | | X | X | X | | |
| 47 | X | | | | X | X | | X | X | | |
| 48 | X | | | | X | | X | X | X | | |
| 49 | X | | | | | X | X | X | X | | |
| 50 | X | | X | X | X | | | X | X | | |
| 51 | X | | X | X | | X | | X | X | | |
| 52 | X | | X | X | | | X | X | X | | |
| 53 | X | | X | | X | X | | X | X | | |
| 54 | X | | X | | X | | X | X | X | | |
| 55 | X | | X | | | X | X | X | X | | |
| 56 | X | | | X | X | X | | X | X | | |
| 57 | X | | | X | X | | X | X | X | | |
| 58 | X | | | | X | X | X | X | X | | |
| 59 | X | | X | X | X | X | | X | X | | |
| 60 | X | | X | X | X | | X | X | X | | |
| 61 | X | | X | | X | X | X | X | X | | |
| 62 | X | | X | X | | X | X | X | X | | |
| 63 | X | | X | | X | X | X | X | X | | |
| 64 | | | | X | X | X | X | X | X | | |
| 65 | X | | X | X | X | X | X | X | X | | |
| 66 | | X | X | | | | | | X | | |
| 67 | | X | | X | | | | | X | | |
| 68 | | X | | | X | | | | X | | |
| 69 | | X | | | | X | | | X | | |
| 70 | | X | | | | | X | | X | | |
| 71 | | X | X | X | | | | | X | | |
| 72 | | X | X | | X | | | | X | | |
| 73 | | X | X | | | X | | | X | | |
| 74 | | X | X | | | | X | | X | | |
| 75 | | X | | X | X | | | | X | | |
| 76 | | X | | X | | X | | | X | | |
| 77 | | X | | X | | | X | | X | | |
| 78 | | X | | | X | X | | | X | | |
| 79 | | X | | | X | | X | | X | | |
| 80 | | X | | | | X | X | | X | | |
| 81 | | X | X | X | X | | | | X | | |
| 82 | | X | X | X | | X | | | X | | |
| 83 | | X | X | X | | | X | | X | | |
| 84 | | X | X | | X | X | | | X | | |
| 85 | | X | X | | X | | X | | X | | |
| 86 | | X | X | | | X | X | | X | | |
| 87 | | X | | X | X | X | | | X | | |
| 88 | | X | | X | X | | X | | X | | |
| 89 | | X | | | X | X | X | | X | | |
| 90 | | X | X | X | X | X | | | X | | |
| 91 | | X | X | X | X | | X | | X | | |
| 92 | | X | X | | X | X | X | | X | | |
| 93 | | X | X | X | | X | X | | X | | |
| 94 | | X | X | | X | X | X | | X | | |
| 95 | | X | | X | X | X | X | | X | | |
| 96 | | X | X | X | X | X | X | | X | | |
| 97 | | X | X | | | | | X | X | | |
| 98 | | X | | X | | | | X | X | | |
| 99 | | X | | | X | | | X | X | | |
| 100 | | X | | | | X | | X | X | | |
| 101 | | X | | | | | X | X | X | | |
| 102 | | X | X | X | | | | X | X | | |
| 103 | | X | X | | X | | | X | X | | |
| 104 | | X | X | | | X | | X | X | | |
| 105 | | X | X | | | | X | X | X | | |
| 106 | | X | | X | X | | | X | X | | |
| 107 | | X | | X | | X | | X | X | | |
| 108 | | X | | X | | | X | X | X | | |
| 109 | | X | | | X | X | | X | X | | |
| 110 | | X | | | X | | X | X | X | | |
| 111 | | X | | | | X | X | X | X | | |
| 112 | | X | X | X | X | | | X | X | | |
| 113 | | X | X | X | | X | | X | X | | |
| 114 | | X | X | X | | | X | X | X | | |
| 115 | | X | X | | X | X | | X | X | | |
| 116 | | X | X | | X | | X | X | X | | |
| 117 | | X | X | | | X | X | X | X | | |
| 118 | | X | | X | X | X | | X | X | | |
| 119 | | X | | X | X | | X | X | X | | |
| 120 | | X | | | X | X | X | X | X | | |
| 121 | | X | X | X | X | X | | X | X | | |
| 122 | | X | X | X | X | | X | X | X | | |
| 123 | | X | X | | X | X | X | X | X | | |
| 124 | | X | X | X | | X | X | X | X | | |
| 125 | | X | X | | X | X | X | X | X | | |
| 126 | | X | | X | X | X | X | X | X | | |
| 127 | | X | X | X | X | X | X | X | X | | |
| 128 | X | X | X | | | | | | X | | |
| 129 | X | X | | X | | | | | X | | |
| 130 | X | X | | | X | | | | X | | |
| 131 | X | X | | | | X | | | X | | |
| 132 | X | X | | | | | X | | X | | |
| 133 | X | X | X | X | | | | | X | | |
| 134 | X | X | X | | X | | | | X | | |
| 135 | X | X | X | | | X | | | X | | |
| 136 | X | X | X | | | | X | | X | | |
| 137 | X | X | | X | X | | | | X | | |
| 138 | X | X | | X | | X | | | X | | |
| 139 | X | X | | X | | | X | | X | | |
| 140 | X | X | | | X | X | | | X | | |
| 141 | X | X | | | X | | X | | X | | |
| 142 | X | X | | | | X | X | | X | | |
| 143 | X | X | X | X | X | | | | X | | |
| 144 | X | X | X | X | | X | | | X | | |
| 145 | X | X | X | X | | | X | | X | | |
| 146 | X | X | X | | X | X | | | X | | |
| 147 | X | X | X | | X | | X | | X | | |
| 148 | X | X | X | | | X | X | | X | | |
| 149 | X | X | | X | X | X | | | X | | |
| 150 | X | X | | X | X | | X | | X | | |
| 151 | X | X | | | X | X | X | | X | | |
| 152 | X | X | X | X | X | X | | | X | | |
| 153 | X | X | X | X | X | | X | | X | | |
| 154 | X | X | X | | X | X | X | | X | | |
| 155 | X | X | X | X | | X | X | | X | | |
| 156 | X | X | X | | X | X | X | | X | | |
| 157 | X | X | | X | X | X | X | | X | | |
| 158 | X | X | X | X | X | X | X | | X | | |
| 159 | X | X | X | | | | | X | X | | |
| 160 | X | X | | X | | | | X | X | | |
| 161 | X | X | | | X | | | X | X | | |
| 162 | X | X | | | | X | | X | X | | |
| 163 | X | X | | | | | X | X | X | | |
| 164 | X | X | X | X | | | | X | X | | |
| 165 | X | X | X | | X | | | X | X | | |
| 166 | X | X | X | | | X | | X | X | | |
| 167 | X | X | X | | | | X | X | X | | |
| 168 | X | X | | X | X | | | X | X | | |
| 169 | X | X | | X | | X | | X | X | | |
| 170 | X | X | | X | | | X | X | X | | |
| 171 | X | X | | | X | X | | X | X | | |
| 172 | X | X | | | X | | X | X | X | | |
| 173 | X | X | | | | X | X | X | X | | |
| 174 | X | X | X | X | X | | | X | X | | |
| 175 | X | X | X | X | | X | | X | X | | |
| 176 | X | X | X | X | | | X | X | X | | |
| 177 | X | X | X | | X | X | | X | X | | |
| 178 | X | X | X | | X | | X | X | X | | |
| 179 | X | X | X | | | X | X | X | X | | |
| 180 | X | X | | X | X | X | | X | X | | |
| 181 | X | X | | X | X | | X | X | X | | |
| 182 | X | X | | | X | X | X | X | X | | |
| 183 | X | X | X | X | X | X | | X | X | | |
| 184 | X | X | X | X | X | | X | X | X | | |
| 185 | X | X | X | | X | X | X | X | X | | |
| 186 | X | X | X | X | | X | X | X | X | | |
| 187 | X | X | X | | X | X | X | X | X | | |
| 188 | X | X | | X | X | X | X | X | X | | |
| 189 | X | X | X | X | X | X | X | X | X | | |
| 190 | X | | | | | | | | | X | |
| 191 | | X | | | | | | | | X | |
| 192 | X | X | | | | | | | | X | |
| 193 | X | | X | | | | | | | X | |
| 194 | X | | | X | | | | | | X | |
| 195 | X | | | | X | | | | | X | |
| 196 | X | | | | | X | | | | X | |
| 197 | X | | | | | | X | | | X | |
| 198 | X | | X | X | | | | | | X | |
| 199 | X | | X | | X | | | | | X | |
| 200 | X | | X | | | X | | | | X | |
| 201 | X | | X | | | | X | | | X | |
| 202 | X | | | X | X | | | | | X | |
| 203 | X | | | X | | X | | | | X | |
| 204 | X | | | X | | | X | | | X | |
| 205 | X | | | | X | X | | | | X | |
| 206 | X | | | | X | | X | | | X | |
| 207 | X | | | | | X | X | | | X | |
| 208 | X | | X | X | X | | | | | X | |
| 209 | X | | X | X | | X | | | | X | |
| 210 | X | | X | X | | | X | | | X | |
| 211 | X | | X | | X | X | | | | X | |
| 212 | X | | X | | X | | X | | | X | |
| 213 | X | | X | | | X | X | | | X | |
| 214 | X | | | X | X | X | | | | X | |
| 215 | X | | | X | X | | X | | | X | |
| 216 | X | | | | X | X | X | | | X | |
| 217 | X | | X | X | X | X | | | | X | |
| 218 | X | | X | X | X | | X | | | X | |
| 219 | X | | X | | X | X | X | | | X | |
| 220 | X | | X | X | | X | X | | | X | |
| 221 | X | | X | | X | X | X | | | X | |
| 222 | | | | X | X | X | X | | | X | |
| 223 | X | | X | X | X | X | X | | | X | |
| 224 | X | | X | | | | | X | | X | |
| 225 | X | | | X | | | | X | | X | |
| 226 | X | | | | X | | | X | | X | |
| 227 | X | | | | | X | | X | | X | |
| 228 | X | | | | | | X | X | | X | |
| 229 | X | | X | X | | | | X | | X | |
| 230 | X | | X | | X | | | X | | X | |
| 231 | X | | X | | | X | | X | | X | |
| 232 | X | | X | | | | X | X | | X | |
| 233 | X | | | X | X | | | X | | X | |
| 234 | X | | | X | | X | | X | | X | |
| 235 | X | | | X | | | X | X | | X | |
| 236 | X | | | | X | X | | X | | X | |
| 237 | X | | | | X | | X | X | | X | |
| 238 | X | | | | | X | X | X | | X | |
| 239 | X | | X | X | X | | | X | | X | |
| 240 | X | | X | X | | X | | X | | X | |
| 241 | X | | X | X | | | X | X | | X | |
| 242 | X | | X | | X | X | | X | | X | |
| 243 | X | | X | | X | | X | X | | X | |
| 244 | X | | X | | | X | X | X | | X | |
| 245 | X | | | X | X | X | | X | | X | |
| 246 | X | | | X | X | | X | X | | X | |
| 247 | X | | | | X | X | X | X | | X | |
| 248 | X | | X | X | X | X | | X | | X | |
| 249 | X | | X | X | X | | X | X | | X | |
| 250 | X | | X | | X | X | X | X | | X | |
| 251 | X | | X | X | | X | X | X | | X | |
| 252 | X | | X | | X | X | X | X | | X | |
| 253 | | | | X | X | X | X | X | | X | |
| 254 | X | | X | X | X | X | X | X | | X | |
| 255 | | X | X | | | | | | | X | |
| 256 | | X | | X | | | | | | X | |
| 257 | | X | | | X | | | | | X | |
| 258 | | X | | | | X | | | | X | |
| 259 | | X | | | | | X | | | X | |
| 260 | | X | X | X | | | | | | X | |
| 261 | | X | X | | X | | | | | X | |
| 262 | | X | X | | | X | | | | X | |
| 263 | | X | X | | | | X | | | X | |
| 264 | | X | | X | X | | | | | X | |
| 265 | | X | | X | | X | | | | X | |
| 266 | | X | | X | | | X | | | X | |
| 267 | | X | | | X | X | | | | X | |
| 268 | | X | | | X | | X | | | X | |
| 269 | | X | | | | X | X | | | X | |
| 270 | | X | X | X | X | | | | | X | |
| 271 | | X | X | X | | X | | | | X | |
| 272 | | X | X | X | | | X | | | X | |
| 273 | | X | X | | X | X | | | | X | |
| 274 | | X | X | | X | | X | | | X | |
| 275 | | X | X | | | X | X | | | X | |
| 276 | | X | | X | X | X | | | | X | |
| 277 | | X | | X | X | | X | | | X | |
| 278 | | X | | | X | X | X | | | X | |
| 279 | | X | X | X | X | X | | | | X | |
| 280 | | X | X | X | X | | X | | | X | |
| 281 | | X | X | | X | X | X | | | X | |
| 282 | | X | X | X | | X | X | | | X | |
| 283 | | X | X | | X | X | X | | | X | |
| 284 | | X | | X | X | X | X | | | X | |
| 285 | | X | X | X | X | X | X | | | X | |
| 286 | | X | X | | | | | X | | X | |
| 287 | | X | | X | | | | X | | X | |
| 288 | | X | | | X | | | X | | X | |
| 289 | | X | | | | X | | X | | X | |
| 290 | | X | | | | | X | X | | X | |
| 291 | | X | X | X | | | | X | | X | |
| 292 | | X | X | | X | | | X | | X | |
| 293 | | X | X | | | X | | X | | X | |
| 294 | | X | X | | | | X | X | | X | |
| 295 | | X | | X | X | | | X | | X | |
| 296 | | X | | X | | X | | X | | X | |
| 297 | | X | | X | | | X | X | | X | |
| 298 | | X | | | X | X | | X | | X | |
| 299 | | X | | | X | | X | X | | X | |
| 300 | | X | | | | X | X | X | | X | |
| 301 | | X | X | X | X | | | X | | X | |
| 302 | | X | X | X | | X | | X | | X | |
| 303 | | X | X | X | | | X | X | | X | |
| 304 | | X | X | | X | X | | X | | X | |
| 305 | | X | X | | X | | X | X | | X | |
| 306 | | X | X | | | X | X | X | | X | |
| 307 | | X | | X | X | X | | X | | X | |
| 308 | | X | | X | X | | X | X | | X | |
| 309 | | X | | | X | X | X | X | | X | |
| 310 | | X | X | X | X | X | | X | | X | |
| 311 | | X | X | X | X | | X | X | | X | |
| 312 | | X | X | | X | X | X | X | | X | |
| 313 | | X | X | X | | X | X | X | | X | |
| 314 | | X | X | | X | X | X | X | | X | |
| 315 | | X | | X | X | X | X | X | | X | |
| 316 | | X | X | X | X | X | X | X | | X | |
| 317 | X | X | X | | | | | | | X | |
| 318 | X | X | | X | | | | | | X | |
| 319 | X | X | | | X | | | | | X | |
| 320 | X | X | | | | X | | | | X | |
| 321 | X | X | | | | | X | | | X | |
| 322 | X | X | X | X | | | | | | X | |
| 323 | X | X | X | | X | | | | | X | |
| 324 | X | X | X | | | X | | | | X | |
| 325 | X | X | X | | | | X | | | X | |
| 326 | X | X | | X | X | | | | | X | |
| 327 | X | X | | X | | X | | | | X | |
| 328 | X | X | | X | | | X | | | X | |
| 329 | X | X | | | X | X | | | | X | |
| 330 | X | X | | | X | | X | | | X | |
| 331 | X | X | | | | X | X | | | X | |
| 332 | X | X | X | X | X | | | | | X | |
| 333 | X | X | X | X | | X | | | | X | |
| 334 | X | X | X | X | | | X | | | X | |
| 335 | X | X | X | | X | X | | | | X | |
| 336 | X | X | X | | X | | X | | | X | |
| 337 | X | X | X | | | X | X | | | X | |
| 338 | X | X | | X | X | X | | | | X | |
| 339 | X | X | | X | X | | X | | | X | |
| 340 | X | X | | | X | X | X | | | X | |
| 341 | X | X | X | X | X | X | | | | X | |
| 342 | X | X | X | X | X | | X | | | X | |
| 343 | X | X | X | | X | X | X | | | X | |
| 344 | X | X | X | X | | X | X | | | X | |
| 345 | X | X | X | | X | X | X | | | X | |
| 346 | X | X | | X | X | X | X | | | X | |
| 347 | X | X | X | X | X | X | X | | | X | |
| 348 | X | X | X | | | | | X | | X | |
| 349 | X | X | | X | | | | X | | X | |
| 350 | X | X | | | X | | | X | | X | |
| 351 | X | X | | | | X | | X | | X | |
| 352 | X | X | | | | | X | X | | X | |
| 353 | X | X | X | X | | | | X | | X | |
| 354 | X | X | X | | X | | | X | | X | |
| 355 | X | X | X | | | X | | X | | X | |
| 356 | X | X | X | | | | X | X | | X | |
| 357 | X | X | | X | X | | | X | | X | |
| 358 | X | X | | X | | X | | X | | X | |
| 359 | X | X | | X | | | X | X | | X | |
| 360 | X | X | | | X | X | | X | | X | |
| 361 | X | X | | | X | | X | X | | X | |
| 362 | X | X | | | | X | X | X | | X | |
| 363 | X | X | X | X | X | | | X | | X | |
| 364 | X | X | X | X | | X | | X | | X | |
| 365 | X | X | X | X | | | X | X | | X | |
| 366 | X | X | X | | X | X | | X | | X | |
| 367 | X | X | X | | X | | X | X | | X | |
| 368 | X | X | X | | | X | X | X | | X | |
| 369 | X | X | | X | X | X | | X | | X | |
| 370 | X | X | | X | X | | X | X | | X | |
| 371 | X | X | | | X | X | X | X | | X | |
| 372 | X | X | X | X | X | X | | X | | X | |
| 373 | X | X | X | X | X | | X | X | | X | |
| 374 | X | X | X | | X | X | X | X | | X | |
| 375 | X | X | X | X | | X | X | X | | X | |
| 376 | X | X | X | | X | X | X | X | | X | |
| 377 | X | X | | X | X | X | X | X | | X | |
| 378 | X | X | X | X | X | X | X | X | | X | |
| 379 | X | | | | | | | | X | X | |
| 380 | | X | | | | | | | X | X | |
| 381 | X | X | | | | | | | X | X | |
| 382 | X | | X | | | | | | X | X | |
| 383 | X | | | X | | | | | X | X | |
| 384 | X | | | | X | | | | X | X | |
| 385 | X | | | | | X | | | X | X | |
| 386 | X | | | | | | X | | X | X | |
| 387 | X | | X | X | | | | | X | X | |
| 388 | X | | X | | X | | | | X | X | |
| 389 | X | | X | | | X | | | X | X | |
| 390 | X | | X | | | | X | | X | X | |
| 391 | X | | | X | X | | | | X | X | |
| 392 | X | | | X | | X | | | X | X | |
| 393 | X | | | X | | | X | | X | X | |
| 394 | X | | | | X | X | | | X | X | |
| 395 | X | | | | X | | X | | X | X | |
| 396 | X | | | | | X | X | | X | X | |
| 397 | X | | X | X | X | | | | X | X | |
| 398 | X | | X | X | | X | | | X | X | |
| 399 | X | | X | X | | | X | | X | X | |
| 400 | X | | X | | X | X | | | X | X | |
| 401 | X | | X | | X | | X | | X | X | |
| 402 | X | | X | | | X | X | | X | X | |
| 403 | X | | | X | X | X | | | X | X | |
| 404 | X | | | X | X | | X | | X | X | |
| 405 | X | | | | X | X | X | | X | X | |
| 406 | X | | X | X | X | X | | | X | X | |
| 407 | X | | X | X | X | | X | | X | X | |
| 408 | X | | X | | X | X | X | | X | X | |
| 409 | X | | X | X | | X | X | | X | X | |
| 410 | X | | X | | X | X | X | | X | X | |
| 411 | | | | X | X | X | X | | X | X | |
| 412 | X | | X | X | X | X | X | | X | X | |
| 413 | X | | X | | | | | X | X | X | |
| 414 | X | | | X | | | | X | X | X | |
| 415 | X | | | | X | | | X | X | X | |
| 416 | X | | | | | X | | X | X | X | |
| 417 | X | | | | | | X | X | X | X | |
| 418 | X | | X | X | | | | X | X | X | |
| 419 | X | | X | | X | | | X | X | X | |
| 420 | X | | X | | | X | | X | X | X | |
| 421 | X | | X | | | | X | X | X | X | |
| 422 | X | | | X | X | | | X | X | X | |
| 423 | X | | | X | | X | | X | X | X | |
| 424 | X | | | X | | | X | X | X | X | |
| 425 | X | | | | X | X | | X | X | X | |
| 426 | X | | | | X | | X | X | X | X | |
| 427 | X | | | | | X | X | X | X | X | |
| 428 | X | | X | X | X | | | X | X | X | |
| 429 | X | | X | X | | X | | X | X | X | |
| 430 | X | | X | X | | | X | X | X | X | |
| 431 | X | | X | | X | X | | X | X | X | |
| 432 | X | | X | | X | | X | X | X | X | |
| 433 | X | | X | | | X | X | X | X | X | |
| 434 | X | | | X | X | X | | X | X | X | |
| 435 | X | | | X | X | | X | X | X | X | |
| 436 | X | | | | X | X | X | X | X | X | |
| 437 | X | | X | X | X | X | | X | X | X | |
| 438 | X | | X | X | X | | X | X | X | X | |
| 439 | X | | X | | X | X | X | X | X | X | |
| 440 | X | | X | X | | X | X | X | X | X | |
| 441 | X | | X | | X | X | X | X | X | X | |
| 442 | | | | X | X | X | X | X | X | X | |
| 443 | X | | X | X | X | X | X | X | X | X | |
| 444 | | X | X | | | | | | X | X | |
| 445 | | X | | X | | | | | X | X | |
| 446 | | X | | | X | | | | X | X | |
| 447 | | X | | | | X | | | X | X | |
| 448 | | X | | | | | X | | X | X | |
| 449 | | X | X | X | | | | | X | X | |
| 450 | | X | X | | X | | | | X | X | |
| 451 | | X | X | | | X | | | X | X | |
| 452 | | X | X | | | | X | | X | X | |
| 453 | | X | | X | X | | | | X | X | |
| 454 | | X | | X | | X | | | X | X | |
| 455 | | X | | X | | | X | | X | X | |
| 456 | | X | | | X | X | | | X | X | |
| 457 | | X | | | X | | X | | X | X | |
| 458 | | X | | | | X | X | | X | X | |
| 459 | | X | X | X | X | | | | X | X | |
| 460 | | X | X | X | | X | | | X | X | |
| 461 | | X | X | X | | | X | | X | X | |
| 462 | | X | X | | X | X | | | X | X | |
| 463 | | X | X | | X | | X | | X | X | |
| 464 | | X | X | | | X | X | | X | X | |
| 465 | | X | | X | X | X | | | X | X | |
| 466 | | X | | X | X | | X | | X | X | |
| 467 | | X | | | X | X | X | | X | X | |
| 468 | | X | X | X | X | X | | | X | X | |
| 469 | | X | X | X | X | | X | | X | X | |
| 470 | | X | X | | X | X | X | | X | X | |
| 471 | | X | X | X | | X | X | | X | X | |
| 472 | | X | X | | X | X | X | | X | X | |
| 473 | | X | | X | X | X | X | | X | X | |
| 474 | | X | X | X | X | X | X | | X | X | |
| 475 | | X | X | | | | | X | X | X | |
| 476 | | X | | X | | | | X | X | X | |
| 477 | | X | | | X | | | X | X | X | |
| 478 | | X | | | | X | | X | X | X | |
| 479 | | X | | | | | X | X | X | X | |
| 480 | | X | X | X | | | | X | X | X | |
| 481 | | X | X | | X | | | X | X | X | |
| 482 | | X | X | | | X | | X | X | X | |
| 483 | | X | X | | | | X | X | X | X | |
| 484 | | X | | X | X | | | X | X | X | |
| 485 | | X | | X | | X | | X | X | X | |
| 486 | | X | | X | | | X | X | X | X | |
| 487 | | X | | | X | X | | X | X | X | |
| 488 | | X | | | X | | X | X | X | X | |
| 489 | | X | | | | X | X | X | X | X | |
| 490 | | X | X | X | X | | | X | X | X | |
| 491 | | X | X | X | | X | | X | X | X | |
| 492 | | X | X | X | | | X | X | X | X | |
| 493 | | X | X | | X | X | | X | X | X | |
| 494 | | X | X | | X | | X | X | X | X | |
| 495 | | X | X | | | X | X | X | X | X | |
| 496 | | X | | X | X | X | | X | X | X | |
| 497 | | X | | X | X | | X | X | X | X | |
| 498 | | X | | | X | X | X | X | X | X | |
| 499 | | X | X | X | X | X | | X | X | X | |
| 500 | | X | X | X | X | | X | X | X | X | |
| 501 | | X | X | | X | X | X | X | X | X | |
| 502 | | X | X | X | | X | X | X | X | X | |
| 503 | | X | X | | X | X | X | X | X | X | |
| 504 | | X | | X | X | X | X | X | X | X | |
| 505 | | X | X | X | X | X | X | X | X | X | |
| 506 | X | X | X | | | | | | X | X | |
| 507 | X | X | | X | | | | | X | X | |
| 508 | X | X | | | X | | | | X | X | |
| 509 | X | X | | | | X | | | X | X | |
| 510 | X | X | | | | | X | | X | X | |
| 511 | X | X | X | X | | | | | X | X | |
| 512 | X | X | X | | X | | | | X | X | |
| 513 | X | X | X | | | X | | | X | X | |
| 514 | X | X | X | | | | X | | X | X | |
| 515 | X | X | | X | X | | | | X | X | |
| 516 | X | X | | X | | X | | | X | X | |
| 517 | X | X | | X | | | X | | X | X | |
| 518 | X | X | | | X | X | | | X | X | |
| 519 | X | X | | | X | | X | | X | X | |
| 520 | X | X | | | | X | X | | X | X | |
| 521 | X | X | X | X | X | | | | X | X | |
| 522 | X | X | X | X | | X | | | X | X | |
| 523 | X | X | X | X | | | X | | X | X | |
| 524 | X | X | X | | X | X | | | X | X | |
| 525 | X | X | X | | X | | X | | X | X | |
| 526 | X | X | X | | | X | X | | X | X | |
| 527 | X | X | | X | X | X | | | X | X | |
| 528 | X | X | | X | X | | X | | X | X | |
| 529 | X | X | | | X | X | X | | X | X | |
| 530 | X | X | X | X | X | X | | | X | X | |
| 531 | X | X | X | X | X | | X | | X | X | |
| 532 | X | X | X | | X | X | X | | X | X | |
| 533 | X | X | X | X | | X | X | | X | X | |
| 534 | X | X | X | | X | X | X | | X | X | |
| 535 | X | X | | X | X | X | X | | X | X | |
| 536 | X | X | X | X | X | X | X | | X | X | |
| 537 | X | X | X | | | | | X | X | X | |
| 538 | X | X | | X | | | | X | X | X | |
| 539 | X | X | | | X | | | X | X | X | |
| 540 | X | X | | | | X | | X | X | X | |
| 541 | X | X | | | | | X | X | X | X | |
| 542 | X | X | X | X | | | | X | X | X | |
| 543 | X | X | X | | X | | | X | X | X | |
| 544 | X | X | X | | | X | | X | X | X | |
| 545 | X | X | X | | | | X | X | X | X | |
| 546 | X | X | | X | X | | | X | X | X | |
| 547 | X | X | | X | | X | | X | X | X | |
| 548 | X | X | | X | | | X | X | X | X | |
| 549 | X | X | | | X | X | | X | X | X | |
| 550 | X | X | | | X | | X | X | X | X | |
| 551 | X | X | | | | X | X | X | X | X | |
| 552 | X | X | X | X | X | | | X | X | X | |
| 553 | X | X | X | X | | X | | X | X | X | |
| 554 | X | X | X | X | | | X | X | X | X | |
| 555 | X | X | X | | X | X | | X | X | X | |
| 556 | X | X | X | | X | | X | X | X | X | |
| 557 | X | X | X | | | X | X | X | X | X | |
| 558 | X | X | | X | X | X | | X | X | X | |
| 559 | X | X | | X | X | | X | X | X | X | |
| 560 | X | X | | | X | X | X | X | X | X | |
| 561 | X | X | X | X | X | X | | X | X | X | |
| 562 | X | X | X | X | X | | X | X | X | X | |
| 563 | X | X | X | | X | X | X | X | X | X | |
| 564 | X | X | X | X | | X | X | X | X | X | |
| 565 | X | X | X | | X | X | X | X | X | X | |
| 566 | X | X | | X | X | X | X | X | X | X | |
| 567 | X | X | X | X | X | X | X | X | X | X | |
| 568 | | | | | | | | | | | X |
| 569 | X | | | | | | | | | | X |
| 570 | | X | | | | | | | | | X |
| 571 | X | X | | | | | | | | | X |
| 572 | X | | X | | | | | | | | X |
| 573 | X | | | X | | | | | | | X |
| 574 | X | | | | X | | | | | | X |
| 575 | X | | | | | X | | | | | X |
| 576 | X | | | | | | X | | | | X |
| 577 | X | | X | X | | | | | | | X |
| 578 | X | | X | | X | | | | | | X |
| 579 | X | | X | | | X | | | | | X |
| 580 | X | | X | | | | X | | | | X |
| 581 | X | | | X | X | | | | | | X |
| 582 | X | | | X | | X | | | | | X |
| 583 | X | | | X | | | X | | | | X |
| 584 | X | | | | X | X | | | | | X |
| 585 | X | | | | X | | X | | | | X |
| 586 | X | | | | | X | X | | | | X |
| 587 | X | | X | X | X | | | | | | X |
| 588 | X | | X | X | | X | | | | | X |
| 589 | X | | X | X | | | X | | | | X |
| 590 | X | | X | | X | X | | | | | X |
| 591 | X | | X | | X | | X | | | | X |
| 592 | X | | X | | | X | X | | | | X |
| 593 | X | | | X | X | X | | | | | X |
| 594 | X | | | X | X | | X | | | | X |
| 595 | X | | | | X | X | X | | | | X |
| 596 | X | | X | X | X | X | | | | | X |
| 597 | X | | X | X | X | | X | | | | X |
| 598 | X | | X | | X | X | X | | | | X |
| 599 | X | | X | X | | X | X | | | | X |
| 600 | X | | X | | X | X | X | | | | X |
| 601 | | | | X | X | X | X | | | | X |
| 602 | X | | X | X | X | X | X | | | | X |
| 603 | X | | X | | | | | X | | | X |
| 604 | X | | | X | | | | X | | | X |
| 605 | X | | | | X | | | X | | | X |
| 606 | X | | | | | X | | X | | | X |
| 607 | X | | | | | | X | X | | | X |
| 608 | X | | X | X | | | | X | | | X |
| 609 | X | | X | | X | | | X | | | X |
| 610 | X | | X | | | X | | X | | | X |
| 611 | X | | X | | | | X | X | | | X |
| 612 | X | | | X | X | | | X | | | X |
| 613 | X | | | X | | X | | X | | | X |
| 614 | X | | | X | | | X | X | | | X |
| 615 | X | | | | X | X | | X | | | X |
| 616 | X | | | | X | | X | X | | | X |
| 617 | X | | | | | X | X | X | | | X |
| 618 | X | | X | X | X | | | X | | | X |
| 619 | X | | X | X | | X | | X | | | X |
| 620 | X | | X | X | | | X | X | | | X |
| 621 | X | | X | | X | X | | X | | | X |
| 622 | X | | X | | X | | X | X | | | X |
| 623 | X | | X | | | X | X | X | | | X |
| 624 | X | | | X | X | X | | X | | | X |
| 625 | X | | | X | X | | X | X | | | X |
| 626 | X | | | | X | X | X | X | | | X |
| 627 | X | | X | X | X | X | | X | | | X |
| 628 | X | | X | X | X | | X | X | | | X |
| 629 | X | | X | | X | X | X | X | | | X |
| 630 | X | | X | X | | X | X | X | | | X |
| 631 | X | | X | | X | X | X | X | | | X |
| 632 | | | | X | X | X | X | X | | | X |
| 633 | X | | X | X | X | X | X | X | | | X |
| 634 | | X | X | | | | | | | | X |
| 635 | | X | | X | | | | | | | X |
| 636 | | X | | | X | | | | | | X |
| 637 | | X | | | | X | | | | | X |
| 638 | | X | | | | | X | | | | X |
| 639 | | X | X | X | | | | | | | X |
| 640 | | X | X | | X | | | | | | X |
| 641 | | X | X | | | X | | | | | X |
| 642 | | X | X | | | | X | | | | X |
| 643 | | X | | X | X | | | | | | X |
| 644 | | X | | X | | X | | | | | X |
| 645 | | X | | X | | | X | | | | X |
| 646 | | X | | | X | X | | | | | X |
| 647 | | X | | | X | | X | | | | X |
| 648 | | X | | | | X | X | | | | X |
| 649 | | X | X | X | X | | | | | | X |
| 650 | | X | X | X | | X | | | | | X |
| 651 | | X | X | X | | | X | | | | X |
| 652 | | X | X | | X | X | | | | | X |
| 653 | | X | X | | X | | X | | | | X |
| 654 | | X | X | | | X | X | | | | X |
| 655 | | X | | X | X | X | | | | | X |
| 656 | | X | | X | X | | X | | | | X |
| 657 | | X | | | X | X | X | | | | X |
| 658 | | X | X | X | X | X | | | | | X |
| 659 | | X | X | X | X | | X | | | | X |
| 660 | | X | X | | X | X | X | | | | X |
| 661 | | X | X | X | | X | X | | | | X |
| 662 | | X | X | | X | X | X | | | | X |
| 663 | | X | | X | X | X | X | | | | X |
| 664 | | X | X | X | X | X | X | | | | X |
| 665 | | X | X | | | | | X | | | X |
| 666 | | X | | X | | | | X | | | X |
| 667 | | X | | | X | | | X | | | X |
| 668 | | X | | | | X | | X | | | X |
| 669 | | X | | | | | X | X | | | X |
| 670 | | X | X | X | | | | X | | | X |
| 671 | | X | X | | X | | | X | | | X |
| 672 | | X | X | | | X | | X | | | X |
| 673 | | X | X | | | | X | X | | | X |
| 674 | | X | | X | X | | | X | | | X |
| 675 | | X | | X | | X | | X | | | X |
| 676 | | X | | X | | | X | X | | | X |
| 677 | | X | | | X | X | | X | | | X |
| 678 | | X | | | X | | X | X | | | X |
| 679 | | X | | | | X | X | X | | | X |
| 680 | | X | X | X | X | | | X | | | X |
| 681 | | X | X | X | | X | | X | | | X |
| 682 | | X | X | X | | | X | X | | | X |
| 683 | | X | X | | X | X | | X | | | X |
| 684 | | X | X | | X | | X | X | | | X |
| 685 | | X | X | | | X | X | X | | | X |
| 686 | | X | | X | X | X | | X | | | X |
| 687 | | X | | X | X | | X | X | | | X |
| 688 | | X | | | X | X | X | X | | | X |
| 689 | | X | X | X | X | X | | X | | | X |
| 690 | | X | X | X | X | | X | X | | | X |
| 691 | | X | X | | X | X | X | X | | | X |
| 692 | | X | X | X | | X | X | X | | | X |
| 693 | | X | X | | X | X | X | X | | | X |
| 694 | | X | | X | X | X | X | X | | | X |
| 695 | | X | X | X | X | X | X | X | | | X |
| 696 | X | X | X | | | | | | | | X |
| 697 | X | X | | X | | | | | | | X |
| 698 | X | X | | | X | | | | | | X |
| 699 | X | X | | | | X | | | | | X |
| 700 | X | X | | | | | X | | | | X |
| 701 | X | X | X | X | | | | | | | X |
| 702 | X | X | X | | X | | | | | | X |
| 703 | X | X | X | | | X | | | | | X |
| 704 | X | X | X | | | | X | | | | X |
| 705 | X | X | | X | X | | | | | | X |
| 706 | X | X | | X | | X | | | | | X |
| 707 | X | X | | X | | | X | | | | X |
| 708 | X | X | | | X | X | | | | | X |
| 709 | X | X | | | X | | X | | | | X |
| 710 | X | X | | | | X | X | | | | X |
| 711 | X | X | X | X | X | | | | | | X |
| 712 | X | X | X | X | | X | | | | | X |
| 713 | X | X | X | X | | | X | | | | X |
| 714 | X | X | X | | X | X | | | | | X |
| 715 | X | X | X | | X | | X | | | | X |
| 716 | X | X | X | | | X | X | | | | X |
| 717 | X | X | | X | X | X | | | | | X |
| 718 | X | X | | X | X | | X | | | | X |
| 719 | X | X | | | X | X | X | | | | X |
| 720 | X | X | X | X | X | X | | | | | X |
| 721 | X | X | X | X | X | | X | | | | X |
| 722 | X | X | X | | X | X | X | | | | X |
| 723 | X | X | X | X | | X | X | | | | X |
| 724 | X | X | X | | X | X | X | | | | X |
| 725 | X | X | | X | X | X | X | | | | X |
| 726 | X | X | X | X | X | X | X | | | | X |
| 727 | X | X | X | | | | | X | | | X |
| 728 | X | X | | X | | | | X | | | X |
| 729 | X | X | | | X | | | X | | | X |
| 730 | X | X | | | | X | | X | | | X |
| 731 | X | X | | | | | X | X | | | X |
| 732 | X | X | X | X | | | | X | | | X |
| 733 | X | X | X | | X | | | X | | | X |
| 734 | X | X | X | | | X | | X | | | X |
| 735 | X | X | X | | | | X | X | | | X |
| 736 | X | X | | X | X | | | X | | | X |
| 737 | X | X | | X | | X | | X | | | X |
| 738 | X | X | | X | | | X | X | | | X |
| 739 | X | X | | | X | X | | X | | | X |
| 740 | X | X | | | X | | X | X | | | X |
| 741 | X | X | | | | X | X | X | | | X |
| 742 | X | X | X | X | X | | | X | | | X |
| 743 | X | X | X | X | | X | | X | | | X |
| 744 | X | X | X | X | | | X | X | | | X |
| 745 | X | X | X | | X | X | | X | | | X |
| 746 | X | X | X | | X | | X | X | | | X |
| 747 | X | X | X | | | X | X | X | | | X |
| 748 | X | X | | X | X | X | | X | | | X |
| 749 | X | X | | X | X | | X | X | | | X |
| 750 | X | X | | | X | X | X | X | | | X |
| 751 | X | X | X | X | X | X | | X | | | X |
| 752 | X | X | X | X | X | | X | X | | | X |
| 753 | X | X | X | | X | X | X | X | | | X |
| 754 | X | X | X | X | | X | X | X | | | X |
| 755 | X | X | X | | X | X | X | X | | | X |
| 756 | X | X | | X | X | X | X | X | | | X |
| 757 | X | X | X | X | X | X | X | X | | | X |

In some embodiments, an MG29-1 effector protein can comprise a combination of the R1192 mutation with mutations of residues E172, N577, and K583 of MG29-1 to produce effector proteins with increased nickase activity. In particular, mutation of residues E172, N577, and K583 of MG29-1 to have E172R, N577R, and K583R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into MG29-1 effector (nickase) proteins.

### Example 13: ErCas12a (MAD7) Effector Protein Variants (Nucleases) with Increased Activity

The present example describes ErCas12a effector proteins (nucleases) comprising one or more substitutions at certain residues with increased activity compared to wild-type ErCas12a proteins.

The disclosure contemplates that certain amino acid residues of an ErCas12a effector protein may be substituted (or mutated) to generate ErCas12a effector proteins with increased activity(ies). For example, the present disclosure describes that I524 and/or F840 residues of an amino acid sequence provided in SEQ ID NO: 15 can be substituted (or mutated). The present disclosure also describes that other residues of an amino acid sequence provided in SEQ ID NO: 15 can be substituted (or mutated) in various combinations.

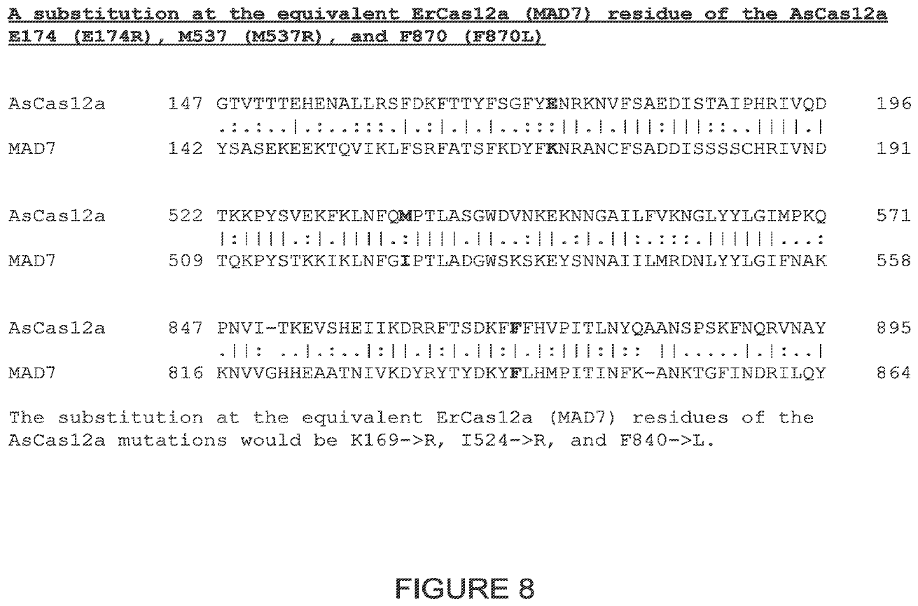

An AsCas12a effector protein comprising two or more substitutions (e.g., M537R and F870L, e.g., SEQ ID NO: 6) has been demonstrated to result in increased activity compared to wild-type AsCas12a (SEQ ID NO: 1) (e.g., see Zhang et al., Nature Communications, 12:3908 (2021)). It is an insight of the present disclosure that ErCas12a (MAD7) effector proteins and AsCas12a effector proteins share sequence conservation of domains (see FIG. 8). Without wishing to be bound by any theory, the present example provides ErCas12a effector proteins with increased activity that are achieved by mutating residues in these conserved regions. For example, without wishing to be bound by any theory, the present example describes that mutating residues I524 and F840 of ErCas12a (MAD7) is expected to produce ErCas12a effector proteins with higher activity compared to wild-type ErCas12a effector proteins. In particular, mutating residues I524 and F840 of ErCas12a to have I524R and F840L mutations, which correspond to M537R and F870L in an exemplary AsCas12a protein (SEQ ID NO: 6), is expected to result in a higher activity ErCas12a relative to the wild-type protein.

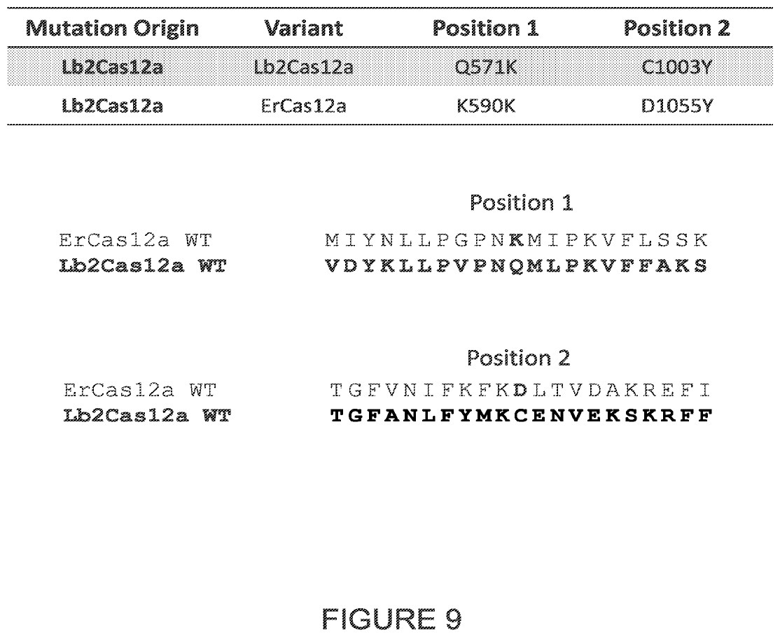

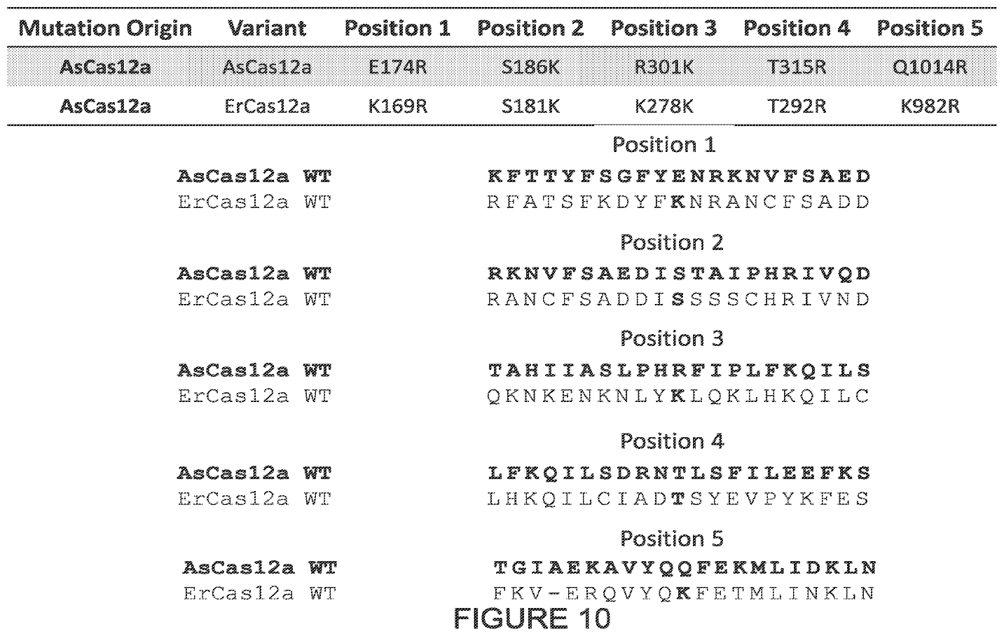

Additional combinations of amino acid substitutions (mutations) relative to exemplary ErCas12a wild-type amino acid sequence SEQ ID NO: 15 are shown in Table 18. Alignments of ErCas12a sequences with corresponding AsCas12a or Lb2Cas12a sequences and exemplary substitutions as described herein are provided in FIGS. 9-10. In some cases, an ErCas12a effector protein can comprise or consist of any of the combinations of amino acid substitutions present in Table 6 relative to an exemplary ErCas12a wild-type effector protein (SEQ ID NO: 15).

TABLE 18
ErCas12a Variants with substitutions
relative to SEQ ID NO: 15

| | I524R | F840L | K169R | D1055Y | S181K | T292R | K982R |
|---|---|---|---|---|---|---|---|
| 1 | X | X | | | | | |
| 2 | | | X | | | | |
| 3 | | | | X | | | |
| 4 | | | X | X | | | |
| 5 | X | X | X | | | | |
| 6 | X | X | | X | | | |
| 7 | X | X | X | X | | | |
| 8 | | | | | X | | |
| 9 | | | | | | X | |
| 10 | | | | | | | X |
| 11 | | | | | X | X | |
| 12 | | | | | X | | X |
| 13 | | | | | | X | X |
| 14 | | | | | X | X | X |
| 15 | X | X | | | X | | |
| 16 | X | X | | | | X | |
| 17 | X | X | | | | | X |
| 18 | X | X | | | X | X | |
| 19 | X | X | | | X | | X |
| 20 | X | X | | | | X | X |
| 21 | X | X | | | X | X | X |
| 22 | X | X | X | | X | X | X |
| 23 | X | X | | X | X | X | X |
| 24 | X | X | X | X | X | X | X |

It is contemplated that amino acid substitutions that can be made are not limited by the present disclosure, and other substitutions can be incorporated into ErCas12a effector (nuclease) proteins.

### Example 14: ErCas12a (MAD7) Effector Protein Variants (Nickases) with Increased Activity

The present example describes ErCas12a effector proteins (nickases) comprising one or more substitutions at certain residues with increased activity compared to wild-type ErCas12a proteins.

The disclosure contemplates that certain amino acid residues of an ErCas12a effector protein may be substituted (or mutated) to generate ErCas12a effector proteins that are nickases, optionally with increased activity(ies). For example, the present disclosure describes that K969 and/or K970 residues of an amino acid sequence provided in SEQ ID NO: 15 can be substituted (or mutated).

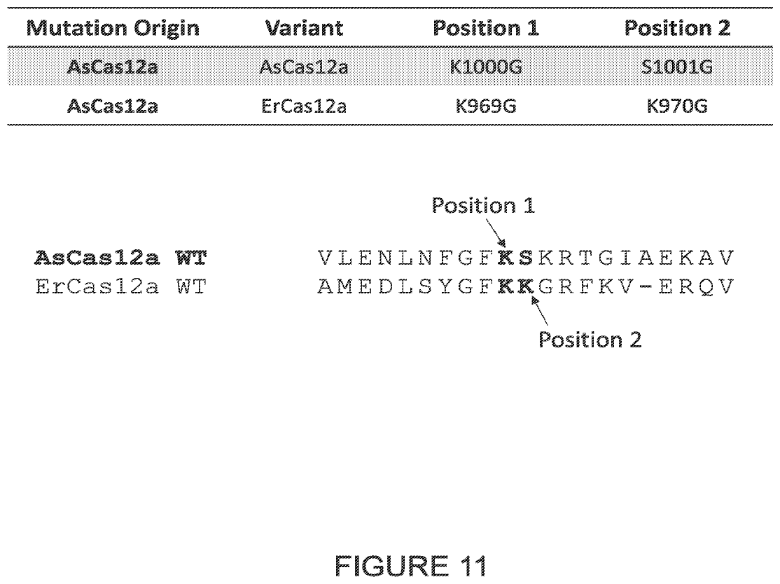

An AsCas12a effector protein comprising two or more substitutions (e.g., K1000G and S1001G) has been demonstrated to result in a nickase version of wild-type AsCas12a (SEQ ID NO: 1). It is an insight of the present disclosure that ErCas12a (MAD7) effector proteins and AsCas12a effector proteins share sequence conservation around these positions (see FIG. 11). Without wishing to be bound by any theory, the present example provides ErCas12a effector proteins with nickase activity by mutating residues in these conserved regions. For example, without wishing to be bound by any theory, the present example describes that mutating residues K969 and/or K970 of ErCas12a (MAD7) is expected to produce ErCas12a effector proteins with nickase activity. In particular, mutations residues K969 and/or K970 of ErCas12a to have K969G and/or K970G, which correspond to K100G and S1001G in an exemplary AsCas12a effector protein (SEQ ID NO: 6), is expected to result in an ErCas12a effector protein with nickase activity.

An AsCas12a effector protein comprising a substitution (e.g., R1226A) has been demonstrated to result in a nickase version of an AsCas12a effector protein (SEQ ID NO: 83). It is an insight of the present disclosure that ErCas12A effector proteins and AsCas12a effector proteins share sequence conservation around these positions (see FIG. 5). Without wishing to be bound by any theory, the present example provides ErCas12A effector proteins with nickase activity by mutating residues in these conserved regions. For example, without wishing to be bound by any theory, the present example describes that mutating a residue R1173 of ErCas12a is expected to produce ErCas12a effector proteins with nickase activity. In particular, mutation of residue R1173 of ErCas12a to have R1173A, which correspond to R1226A in an exemplary AsCas12a effector protein (SEQ ID NO: 1), is expected to result in an MG29-1 effector protein with nickase activity.

Additional combinations of these nickase-inducing mutations with amino acid substitutions (mutations) that increase activity relative to exemplary ErCas12a wild-type amino acid sequence SEQ ID NO: 15 are shown in Table 19. Alignments of ErCas12a sequences with corresponding AsCas12a or Lb2Cas12a sequences and exemplary substitutions as described herein are provided in FIGS. 9-10. In some cases, an ErCas12a effector protein can comprise or consist of any of the combinations of amino acid substitutions present in Table 19 relative to an exemplary ErCas12a wild-type effector protein (SEQ ID NO: 15).

TABLE 19
ErCas12a Variants with substitutions relative to SEQ ID NO: 15

| | I524R | F840L | K169R | D1055Y | S181K | T292R | K982R | K969G | K970G | R1173A |
|---|---|---|---|---|---|---|---|---|---|---|
| 1 | | | | | | | | X | | |
| 2 | | | | | | | | | X | |
| 3 | | | | | | | | X | X | |
| 4 | X | X | | | | | | X | | |
| 5 | X | X | | | | | | | X | |
| 6 | X | X | | | | | | X | X | |
| 7 | | | X | | | | | X | | |
| 8 | | | X | | | | | | X | |
| 9 | | | X | | | | | X | X | |
| 10 | | | | X | | | | X | | |
| 11 | | | | X | | | | | X | |
| 12 | | | | X | | | | X | X | |
| 13 | X | X | X | | | | | X | | |
| 14 | X | X | X | | | | | | X | |
| 15 | X | X | X | | | | | X | X | |
| 16 | X | X | | X | | | | X | | |
| 17 | X | X | | X | | | | | X | |
| 18 | X | X | | X | | | | X | X | |
| 19 | X | X | X | X | | | | X | | |
| 20 | X | X | X | X | | | | | X | |
| 21 | X | X | X | X | | | | X | X | |
| 22 | | | | | X | | | X | | |
| 23 | | | | | X | | | | X | |
| 24 | | | | | X | | | X | X | |
| 25 | | | | | | X | | X | | |
| 26 | | | | | | X | | | X | |
| 27 | | | | | | X | | X | X | |
| 28 | | | | | | | X | X | | |
| 29 | | | | | | | X | | X | |
| 30 | | | | | | | X | X | X | |
| 31 | | | | | X | X | | X | | |
| 32 | | | | | X | X | | | X | |
| 33 | | | | | X | X | | X | X | |
| 34 | | | | | X | | X | X | | |
| 35 | | | | | X | | X | | X | |
| 36 | | | | | X | | X | X | X | |
| 37 | | | | | | X | X | X | | |
| 38 | | | | | | X | X | | X | |
| 39 | | | | | | X | X | X | X | |
| 40 | | | | | X | X | X | X | | |
| 41 | | | | | X | X | X | | X | |
| 42 | | | | | X | X | X | X | X | |
| 43 | X | X | | | X | | | X | | |
| 44 | X | X | | | X | | | | X | |
| 45 | X | X | | | X | | | X | X | |
| 46 | X | X | | | | X | | X | | |
| 47 | X | X | | | | X | | | X | |
| 48 | X | X | | | | X | | X | X | |
| 49 | X | X | | | | | X | X | | |
| 50 | X | X | | | | | X | | X | |
| 51 | X | X | | | | | X | X | X | |
| 52 | X | X | | | X | X | | X | | |
| 53 | X | X | | | X | X | | | X | |
| 54 | X | X | | | X | X | | X | X | |
| 55 | X | X | | | X | | X | X | | |
| 56 | X | X | | | X | | X | | X | |
| 57 | X | X | | | X | | X | X | X | |
| 58 | X | X | | | | X | X | X | | |
| 59 | X | X | | | | X | X | | X | |
| 60 | X | X | | | | X | X | X | X | |
| 61 | X | X | | | X | X | X | X | | |
| 62 | X | X | | | X | X | | | X | |
| 63 | X | X | | | | X | X | X | X | |
| 64 | X | X | X | | X | X | X | X | | |
| 65 | X | X | X | | X | X | X | | X | |
| 66 | X | X | X | | X | X | X | X | X | |
| 67 | X | X | | X | X | X | X | X | | |
| 68 | X | X | | X | X | X | X | | X | |
| 69 | X | X | | X | X | X | X | X | X | |
| 70 | X | X | X | X | X | X | X | X | | |
| 71 | X | X | X | X | X | X | X | | X | |
| 72 | X | X | X | X | X | X | X | X | X | |
| 73 | | | | | | | | | | X |
| 74 | X | X | | | | | | | | X |
| 75 | | | X | | | | | | | X |
| 76 | | | | X | | | | | | X |
| 77 | X | X | X | | | | | | | X |
| 78 | X | X | | X | | | | | | X |
| 79 | X | X | X | X | | | | | | X |
| 80 | | | | | X | | | | | X |
| 81 | | | | | | X | | | | X |
| 82 | | | | | | | X | | | X |
| 83 | | | | | X | X | | | | X |
| 84 | | | | | X | | X | | | X |
| 85 | | | | | | X | X | | | X |
| 86 | | | | | X | X | X | | | X |
| 87 | X | X | | | X | | | | | X |
| 88 | X | X | | | | X | | | | X |
| 89 | X | X | | | | | X | | | X |
| 90 | X | X | | | X | X | | | | X |
| 91 | X | X | | | X | | X | | | X |
| 92 | X | X | | | | X | X | | | X |
| 93 | X | X | | | X | X | X | | | X |
| 94 | X | X | X | | X | X | X | | | X |
| 95 | X | X | | X | X | X | X | | | X |
| 96 | X | X | X | X | X | X | X | | | X |

In some embodiments, an ErCas12a effector protein can comprise a combination of the R1173 mutation with mutations of residues K169, D529, and K535 of ErCas12a to produce effector proteins with increased nickase activity. In particular, mutation of residues K169, D529, and K535 of ErCas12a to have K169R, D529R, and K535R, which correspond to E174R, S542R, and K548R in an exemplary AsCas12a effector protein (SEQ ID NO: 84).

It is contemplated that amino acid substitutions that can be made are not limited by the present disclosure, and other substitutions can be incorporated into ErCas12a effector (nickase) proteins.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

We claim:

1. A Cas12a effector protein comprising:
(a) a polypeptide sequence having one or more amino acid substitutions relative to the sequence of a naturally occurring Cas12a effector protein, wherein the Cas12a effector protein is a nuclease that exhibits increased nuclease activity compared to the naturally occurring Cas12a effector protein; or
(b) a polypeptide sequence having one or more amino acid substitutions relative to the sequence of a nickase version of a naturally occurring Cas12a effector protein, wherein the Cas12a effector protein is a nickase that exhibits increased nickase activity compared to the nickase version of the naturally occurring Cas12a effector protein.

2. The Cas12a effector protein of claim 1, wherein the naturally occurring Cas12a effector protein has a polypeptide sequence of any one of SEQ ID NOs: 1-5 and 14-15.

3. The Cas12a effector protein of claim 1 or 2, wherein the nickase version of the naturally occurring Cas12a effector protein has a substitution at a position corresponding to K1000 and/or S1001 in SEQ ID NO: 1.

4. The Cas12a effector protein of claim 1, wherein the polypeptide sequence comprises a substitution at a position corresponding to N602 of SEQ ID NO: 2, R507 of SEQ ID NO: 3, N527 of SEQ ID NO: 4, N568 of SEQ ID NO: 5, A572 of SEQ ID NO: 14, or I524R of SEQ ID NO: 15.

5. The Cas12a effector protein of claim 1 or 2, wherein the polypeptide sequence comprises a substitution of N602R relative to SEQ ID NO: 2, N527R relative to SEQ ID NO: 4, N568R relative to SEQ ID NO: 5, A572R relative to SEQ ID NO: 14, or I524R relative to SEQ ID NO: 15.

6. The Cas12a effector protein of any one of the preceding claims, wherein the polypeptide sequence comprises a substitution at a position corresponding to F879 of SEQ ID NO: 2, T778 of SEQ ID NO: 3, E795 of SEQ ID NO: 4, M825 of SEQ ID NO: 5, F849 of SEQ ID NO: 14, or F840 of SEQ ID NO: 15.

7. The Cas12a effector protein of any one of the preceding claims, wherein the polypeptide sequence comprises a substitution of F879L relative to SEQ ID NO: 2, T778L relative to SEQ ID NO: 3, E795L relative to SEQ ID NO: 4, M825L relative to SEQ ID NO: 5, F849L relative to SEQ ID NO: 14, or F840L relative to SEQ ID NO: 15.

8. The Cas12a effector protein of any one of the preceding claims, wherein the polypeptide sequence comprises substitutions of N602R and F879L relative to SEQ ID NO: 2, N527R and E795L relative to SEQ ID NO: 4, N568R and M825L relative to SEQ ID NO: 5, A572R and F849L relative to SEQ ID NO: 14, or I524R and F840L relative to SEQ ID NO: 15.

9. The Cas12a effector protein of any one of the preceding claims, wherein the polypeptide sequence comprises a substitution at a position corresponding to E184 of SEQ ID NO: 2, K155 of SEQ ID NO: 3, D156 of SEQ ID NO: 4, D172 of SEQ ID NO: 5, E172 of SEQ ID NO: 14, or K169 of SEQ ID NO: 15.

10. The Cas12a effector protein of any one of the preceding claims, wherein the polypeptide sequence comprises a substitution of E184R relative to SEQ ID NO: 2, K155R relative to SEQ ID NO: 3, D156R relative to SEQ ID NO: 4, D172R relative to SEQ ID NO: 5, E172R relative to SEQ ID NO: 14 or K169R relative to SEQ ID NO: 15.

11. The Cas12a effector protein of any one of the preceding claims, wherein the polypeptide sequence comprises a substitution at a position corresponding to P196 of SEQ ID NO: 2, S167 of SEQ ID NO: 3, S168 of SEQ ID NO: 4, H184 of SEQ ID NO: 5, S184 of SEQ ID NO: 14, or S181 of SEQ ID NO: 15.

12. The Cas12a effector protein of any one of the preceding claims, wherein the polypeptide sequence comprises a substitution of P196K relative to SEQ ID NO: 2, S167K relative to SEQ ID NO: 3, S168K relative to SEQ ID NO: 4, H184K relative to SEQ ID NO: 5, S184K relative to SEQ ID NO: 14, or S181K relative to SEQ ID NO: 15.

13. The Cas12a effector protein of any one of the preceding claims, wherein the polypeptide sequence comprises substitutions at positions corresponding to M537, F870, and R301 of SEQ ID NO: 1 or A572, F849, and R292 of SEQ ID NO: 14.

14. The Cas12a effector protein of any one of the preceding claims, wherein the polypeptide sequence comprises substitutions of M537R, F870L, and R301K relative to SEQ ID NO: 1 or A572R, F849L, and R292K relative to SEQ ID NO: 14.

15. The Cas12a effector protein of any one of the preceding claims, wherein the polypeptide sequence comprises a substitution at a position corresponding to S334 of SEQ ID NO: 2, E271 of SEQ ID NO: 3, S286 of SEQ ID NO: 4, G292 of SEQ ID NO: 5, T306 of SEQ ID NO: 14, or T292 of SEQ ID NO: 15.

16. The Cas12a effector protein of any one of the preceding claims, wherein the polypeptide sequence comprises a substitution of S334R relative to SEQ ID NO: 2, E271R relative to SEQ ID NO: 3, S286R relative to SEQ ID NO: 4, G292R relative to SEQ ID NO: 5, T306R relative to SEQ ID NO: 14, or T292R relative to SEQ ID NO: 15.

17. The Cas12a effector protein of any one of the preceding claims, wherein the polypeptide sequence comprises a substitution at a position corresponding to K1026 of SEQ ID NO: 2, K926 of SEQ ID NO: 3, K945 of SEQ ID NO: 4, N978 of SEQ ID NO: 5, K996 of SEQ ID NO: 14, or K982 of SEQ ID NO: 15.

18. The Cas12a effector protein of any one of the preceding claims, wherein the polypeptide sequence comprises a substitution of K1026R relative to SEQ ID NO: 2, K926R relative to SEQ ID NO: 3, K945R relative to SEQ ID NO: 4, N978R relative to SEQ ID NO: 5, K996R relative to SEQ ID NO: 14, or relative to K982R of SEQ ID NO: 15.

19. The Cas12a effector protein of any one of the preceding claims, wherein the polypeptide sequence comprises a substitution at a position corresponding to I1088 of SEQ ID NO: 1, Y1099 of SEQ ID NO: 2, Y1013 of SEQ ID NO: 4, Y1051 of SEQ ID NO: 5, Y1069 of SEQ ID NO: 14, or D1055 of SEQ ID NO: 15.

20. The Cas12a effector protein of any one of the preceding claims, wherein the polypeptide sequence comprises a substitution of I1088Y relative to SEQ ID NO: 1 or D1055Y relative to SEQ ID NO: 15.

21. The Cas12a effector protein of any one of the preceding claims, wherein the polypeptide sequence comprises substitutions of M537R, F870L, and E174R relative to SEQ ID NO: 1; N602R, F879L, and E184R relative to SEQ ID NO: 2; T778L and K155R relative to SEQ ID NO: 3; N527R, E795L, and D156R relative to SEQ ID NO: 4; N568R, M825L, and D172R relative to SEQ ID NO: 5; A572R, F849L, and E172R relative to SEQ ID NO: 14; or I524R, F840L, and K169R relative to SEQ ID NO: 15.

22. The Cas12a effector protein of any one of the preceding claims, wherein the polypeptide sequence comprises substitutions of M537R, F870L, and S186K relative to SEQ ID NO: 1; N602R, F879L, and P196K relative to SEQ ID NO: 2; T778L and $167K relative to SEQ ID NO: 3; N527R, E795L, and S168K relative to SEQ ID NO: 4; N568R, M825L, and H184K relative to SEQ ID NO: 5; A572R, F849L, and S184K relative to SEQ ID NO: 14; or I524R, F840L, and S181K relative to SEQ ID NO: 15.

23. The Cas12a effector protein of any one of the preceding claims, wherein the polypeptide sequence comprises substitutions of R1226 relative to SEQ ID NO: 1, R1218 relative to SEQ ID NO: 2, R1124 relative to SEQ ID NO: 3, R1138 relative to SEQ ID NO: 4, R1171 relative to SEQ ID NO: 5, R1192 relative to SEQ ID NO: 14, or R1173 relative to SEQ ID NO: 15.

24. The Cas12a effector protein of any one of the preceding claims, wherein the polypeptide sequence comprises substitutions of R1226A relative to SEQ ID NO: 1, R1218A relative to SEQ ID NO: 2, R1124A relative to SEQ ID NO: 3, R1138A relative to SEQ ID NO: 4, R1171A relative to SEQ ID NO: 5, R1192A relative to SEQ ID NO: 14, or R1173A relative to SEQ ID NO: 15.

25. The Cas12a effector protein of any one of the preceding claims, wherein the polypeptide sequence comprises substitutions of E174, S542, K548, and R1226 relative to SEQ ID NO: 1; E184, N607, K613, and R1218 relative to SEQ ID NO: 2; K155, N512, K518, and R1124 relative to SEQ ID NO: 3; D156, G532, K538, and R1138 relative to SEQ ID NO: 4; D172, N563, K569, and R1171 relative to SEQ ID NO: 5; E172, N577, K583, and R1192 relative to SEQ ID NO: 14: or K169, D529, K535, and R1173 relative to SEQ ID NO: 15.

26. The Cas12a effector protein of any one of the preceding claims, wherein the polypeptide sequence comprises substitutions of E174R, S542R, K548R, and R1226A relative to SEQ ID NO: 1; E184R, N607R, K613R, and R1218A relative to SEQ ID NO: 2; K155R, N512R, K518R, and R1124A relative to SEQ ID NO: 3; D156R, G532R, K538R, and R1138A relative to SEQ ID NO: 4; D172R, N563R, K569R, and R1171A relative to SEQ ID NO: 5; E172R, N577R, K583R, and R1192A relative to SEQ ID NO: 14; or K169R, D529R, K535R, and R1173A relative to SEQ ID NO: 15.

27. The Cas12a effector protein of any one of the preceding claims, wherein the polypeptide sequence comprises substitutions of M537R, F870L, and T315R relative to SEQ ID NO: 1; N602R, F879L, and S334R relative to SEQ ID NO: 2; T778L and E271R relative to SEQ ID NO: 3; N527R, E795L, and S286R relative to SEQ ID NO: 4; N568R, M825L, and G292R relative to SEQ ID NO: 5; A572R, F849L, and T306R relative to SEQ ID NO: 14: or I524R, F840L, and T292R relative to SEQ ID NO: 15.

28. The Cas12a effector protein of any one of the preceding claims, wherein the polypeptide sequence comprises substitutions of M537R, F870L, and Q1014R relative to SEQ ID NO: 1; N602R, F879L, and K1026R relative to SEQ ID NO: 2; T778L and K926R relative to SEQ ID NO: 3; N527R, E795L, and K945R relative to SEQ ID NO: 4; N568R, M825L, and N978R relative to SEQ ID NO: 5; A572R, F849L, and K996R relative to SEQ ID NO: 14; or I524R, F840L, and K982R relative to SEQ ID NO: 15.

29. The Cas12a effector protein of any one of the preceding claims, wherein the polypeptide sequence comprises substitutions of M537R, F870L, and I1088Y relative to SEQ ID NO: 1; T778L and C1003Y relative to SEQ ID NO: 3; or I524R, F840L, and D1055Y relative to SEQ ID NO: 15.

30. The Cas12a effector protein of any one of the preceding claims, wherein the polypeptide sequence comprises substitutions of T778L and Q571K relative to SEQ ID NO: 3.

31. The Cas12a effector protein of any one of the preceding claims, wherein the polypeptide sequence comprises substitutions of T778L, Q571K, and C1003Y relative to SEQ ID NO: 3.

32. The Cas12a effector protein of any one of the preceding claims, wherein the polypeptide sequence comprises substitutions of M537R, F870L, E174R, and I1088Y relative to SEQ ID NO: 1; T778L, K155R, and C1003Y relative to SEQ ID NO: 3; or I524R, F840L, K169R, and D1055Y relative to SEQ ID NO: 15.

33. The Cas12a effector protein of any one of the preceding claims, wherein the polypeptide sequence comprises substitutions of S186K and T315R relative to SEQ ID NO: 1; P196K and S334R relative to SEQ ID NO: 2; S167K and E271R relative to SEQ ID NO: 3; S168K and S286R relative to SEQ ID NO: 4; H184K and G292R relative to SEQ ID NO: 5; S184K and T306R relative to SEQ ID NO: 14; or S181K and T292R relative to SEQ ID NO: 15.

34. The Cas12a effector protein of any one of the preceding claims, wherein the polypeptide sequence comprises substitutions of S186K, R301K, and T315R relative to SEQ ID NO: 1 or S184K, R292K, and T306R relative to SEQ ID NO: 14.

35. The Cas12a effector protein of any one of the preceding claims, wherein the polypeptide sequence comprises substitutions of S186K, R301K, and Q1014R relative to SEQ ID NO: 1.

36. The Cas12a effector protein of any one of the preceding claims, wherein the polypeptide sequence comprises substitutions of T315R and Q1014R relative to SEQ ID NO: 1; S334R and K1026R relative to SEQ ID NO: 2; E271R and K926R relative to SEQ ID NO: 3; S286R and K945R relative to SEQ ID NO: 4; G292R and N978R relative to SEQ ID NO: 5; T306R and K996R relative to SEQ ID NO: 14; or T292R and K982R relative to SEQ ID NO: 15.

37. The Cas12a effector protein of any one of the preceding claims, wherein the polypeptide sequence comprises substitutions of S186K and Q1014R relative to SEQ ID NO: 1; P196K and K1026R relative to SEQ ID NO: 2; S167K and K926R relative to SEQ ID NO: 3; S168K and K945R relative to SEQ ID NO: 4; H184K and N978R relative to SEQ ID NO: 5; S184K and K996R relative to SEQ ID NO: 14; or S181K and K982R relative to SEQ ID NO: 15.38. The Cas12a effector protein of any one of the preceding claims, wherein the polypeptide sequence comprises substitutions of S186K, T315R and Q1014R relative to SEQ ID NO: 1; P196K, S334R, and K1026R relative to SEQ ID NO: 2; S167K, E271R, and K926R relative to SEQ ID NO: 3; S168K, S286R, and K945R relative to SEQ ID NO: 4; H184K, G292R, and N978R relative to SEQ ID NO: 5; S184K, T306R and K996R relative to SEQ ID NO: 14; or S181K, T292R, and K982R relative to SEQ ID NO: 15.

38. The Cas12a effector protein of any one of the preceding claims, wherein the polypeptide sequence comprises substitutions of S186K, R301K, T315R and Q1014R relative to SEQ ID NO: 1 or S184K, R292K, T306R and K996R relative to SEQ ID NO: 14.

39. The Cas12a effector protein of any one of the preceding claims, wherein the polypeptide sequence comprises a substitution of S168R relative to SEQ ID NO: 14.

40. The Cas12a effector protein of any one of the preceding claims, wherein the Cas12a effector protein is a nuclease.

41. The Cas12a effector protein of any one of the preceding claims, wherein the Cas12a effector protein is a nickase.

42. The Cas12a effector protein of claim 41, wherein the polypeptide sequence comprises a substitution at a position corresponding to K1000 of SEQ ID NO: 1, K1013 of SEQ ID NO: 2, K913 of SEQ ID NO: 3, K932 of SEQ ID NO: 4, K965 of SEQ ID NO: 5, K983 of SEQ ID NO: 14, or K969 of SEQ ID NO: 15.

43. The Cas12a effector protein of claim 41, wherein the polypeptide sequence comprises a substitution of K1000G relative to SEQ ID NO: 1, K1013G relative to SEQ ID NO: 2, K913G relative to SEQ ID NO: 3, K932G relative to SEQ ID NO: 4, or K965G relative to SEQ ID NO: 5, K983G relative to SEQ ID NO: 14, or K969G relative to SEQ ID NO: 15.

44. The Cas12a effector protein of claim 41, wherein the polypeptide sequence comprises a substitution at a position corresponding to S1001 of SEQ ID NO: 1, R1014 of SEQ ID NO: 2, R914 of SEQ ID NO: 3, N933 of SEQ ID NO: 4, R966 of SEQ ID NO: 5, R984 of SEQ ID NO: 14, or K970 of SEQ ID NO: 15.

45. The Cas12a effector protein of claim 41, wherein the polypeptide sequence comprises a substitution of $1001G relative to SEQ ID NO: 1, R1014G relative to SEQ ID NO: 2, R914G relative to SEQ ID NO: 3, N933G relative to SEQ ID NO: 4, R966G relative to SEQ ID NO: 5, R984G relative to SEQ ID NO: 14, or K970G relative to SEQ ID NO: 15.

46. The Cas12a effector protein of claim 41, wherein the polypeptide sequence comprises substitutions of K1000G and S1001G relative to SEQ ID NO: 1; K1013G and R1014G relative to SEQ ID NO: 2; K913G and R914G relative to SEQ ID NO: 3; K932G and N933G relative to SEQ ID NO: 4; K965G and R966G relative to SEQ ID NO: 5; K983G and R984G relative to SEQ ID NO: 14; or K969G and K970G relative to SEQ ID NO: 15.

47. The Cas12a effector protein of any one of claims 1-46, wherein the naturally occurring Cas12a effector protein has a polypeptide sequence of SEQ ID NO: 1.

48. The Cas12a effector protein of any one of claims 1-46, wherein the naturally occurring Cas12a effector protein has a polypeptide sequence of SEQ ID NO: 2.

49. The Cas12a effector protein of any one of claims 1-46, wherein the naturally occurring Cas12a effector protein has a polypeptide sequence of SEQ ID NO: 3.

50. The Cas12a effector protein of any one of claims 1-46, wherein the naturally occurring Cas12a effector protein has a polypeptide sequence of SEQ ID NO: 4.

51. The Cas12a effector protein of any one of claims 1-46, wherein the naturally occurring Cas12a effector protein has a polypeptide sequence of SEQ ID NO: 5.

52. The Cas12a effector protein of any one of claims 1-46, wherein the naturally occurring Cas12a effector protein has a polypeptide sequence of SEQ ID NO: 14.

53. The Cas12a effector protein of any one of the preceding claims, wherein the polypeptide sequence comprises a substitution at a position corresponding to K535 of SEQ ID NO: 15.

54. The Cas12a effector protein of claim 53, wherein the polypeptide sequence comprises a substitution of K535R relative to SEQ ID NO: 15.

55. The Cas12a effector protein of any one of the preceding claims, wherein the polypeptide sequence comprises a substitution at a position corresponding to K594 of SEQ ID NO: 15.

56. The Cas12a effector protein of claim 55, wherein the polypeptide sequence comprises a substitution of K594L relative to SEQ ID NO: 15.

57. The Cas12a effector protein of any one of the preceding claims, wherein the polypeptide sequence comprises a substitution at a position corresponding to K169 of SEQ ID NO: 15.

58. The Cas12a effector protein of claim 57, wherein the polypeptide sequence comprises a substitution of K169R relative to SEQ ID NO: 15.

59. The Cas12a effector protein of any one of the preceding claims, wherein the polypeptide sequence comprises a substitution at a position corresponding to N264 of SEQ ID NO: 15.

60. The Cas12a effector protein of claim 59, wherein the polypeptide sequence comprises a substitution of N264A relative to SEQ ID NO: 15.

61. The Cas12a effector protein of any one of the preceding claims, wherein the polypeptide sequence comprises a substitution at a position corresponding to D529 of SEQ ID NO: 15

62. The Cas12a effector protein of claim 61, wherein the polypeptide sequence comprises a substitution of D529R relative to SEQ ID NO: 15.

63. The Cas12a effector protein of any one of the preceding claims, wherein the polypeptide sequence comprises a substitution at a position corresponding to K535 of SEQ ID NO: 15.

64. The Cas12a effector protein of claim 63, wherein the polypeptide sequence comprises a substitution of K535V relative to SEQ ID NO: 15.

65. The Cas12a effector protein of claim 64, wherein the polypeptide sequence comprises a substitution of K535R relative to SEQ ID NO: 15.

66. The Cas12a effector protein of any one of the preceding claims, wherein the polypeptide sequence comprises a substitution at a position corresponding to N539 of SEQ ID NO: 15.

67. The Cas12a effector protein of claim 66, wherein the polypeptide sequence comprises a substitution of N539R relative to SEQ ID NO: 15.

68. The Cas12a effector protein of any one of the preceding claims, wherein the polypeptide sequence comprises a substitution at a position corresponding to K594 of SEQ ID NO: 15.

69. The ErCas12a effector protein of claim 68, wherein the polypeptide sequence comprises a substitution of K594R relative to SEQ ID NO: 15.

70. The Cas12a effector protein of any one of the preceding claims, wherein the polypeptide sequence is at least 90% identical to SEQ ID NO: 16

71. The Cas12a effector protein of claim 1, wherein the polypeptide sequence comprises SEQ ID NO: 16.

72. A fusion protein comprising a Cas12a effector protein according to any one of claims 1-71, wherein the Cas12a effector protein is fused to a deaminase, e.g., a cytosine deaminase or an adenine deaminase.

73. A polynucleotide encoding a Cas12a effector protein according to any one of claims 1-71 or a fusion protein according to claim 72.

74. The polynucleotide according to claim 73, wherein the polynucleotide is codon-optimized for expression in a host cell.

75. A recombinant vector comprising a polynucleotide of claim 73 or 74.

76. The recombinant vector of claim 75, wherein the polynucleotide is operably linked to a promoter.

77. The recombinant vector of claim 75 or 76, further comprising a nucleic acid sequence encoding a gRNA molecule.

78. An AAV particle comprising the polynucleotide of claim 73 or 74 or recombinant vector of any one of claims 75-77.

79. A lipid nanoparticle (LNP) comprising the polynucleotide of claim 73 or 74 or recombinant vector of any one of claims 75-77.

80. A cell capable of expressing the Cas12a effector protein of any one of claims 1-71 or the fusion protein according to claim 72.

81. A cell comprising the polynucleotide of any one of claim 73 or 74.

82. A cell comprising the recombinant vector of any one of claims 75-77.

83. A CRISPR/Cas12a effector system comprising:
(a) a polynucleotide of claim 73 or 74, or a recombinant vector of any one of claims 75-77; and
(b) a polynucleotide or a recombinant vector comprising a nucleic acid sequence encoding a gRNA molecule.

84. The CRISPR/Cas12a effector system of claim 83, further comprising
(c) a cell for expression of the polynucleotide or the recombinant vector of (a) and (b).

85. The cell of any one of claims 80-82 or the CRISPR/Cas12a effector system of claim 83 or 84, wherein the cell is a prokaryotic cell or a eukaryotic cell.

86. A composition (e.g., a pharmaceutical composition) comprising the Cas12a effector protein of any one of claims 1-71 and a gRNA molecule, the fusion protein according to claim 72 and a gRNA molecule, the polynucleotide of any one of claims 73-74, the recombinant vector of any one of claims 75-77, the AAV particle of claim 78, the LNP of claim 79, or the cell of any one of claims 80-82.

87. A composition comprising a ribonucleoprotein (RNP) complex comprising the Cas12a effector protein of any one of claims 1-71 and a gRNA molecule or the fusion protein according to claim 72 and a gRNA molecule.

88. A method of genetically engineering a population of cells, the method comprising:
expressing in the cells or contacting the cells with the Cas12a effector protein of any one of claims 1-71 and a gRNA molecule or the fusion protein according to claim 72 and a gRNA molecule, whereby genomes of at least a plurality of the cells are altered.

89. A method of editing a population of double stranded DNA (dsDNA) molecules, the method comprising:
contacting the dsDNA molecules with the Cas12a effector protein of any one of claims 1-71 and a gRNA molecule or the fusion protein according to claim 72 and a gRNA molecule, whereby a plurality of the dsDNA molecules are edited.

90. The method of any one of claims 88-89, wherein the Cas12a effector protein and gRNA molecule or the fusion protein according to claim 72 and a gRNA molecule are administered as a ribonucleoprotein (RNP).

91. A method of treatment comprising:
introducing the Cas12a effector protein of any one of claims 1-71 and a gRNA molecule, the fusion protein according to claim 72 and a gRNA molecule, the polynucleotide of any one of claims 73-74, the recombinant vector of any one of claims 75-77, the AAV particle of claim 78, or the LNP of claim 79 into a subject.

92. Use of the Cas12a effector protein of any one of claims 1-71 and a gRNA molecule, the fusion protein according to claim 72 and a gRNA molecule, the polynucleotide of any one of claims 73-74, the recombinant vector of any one of claims 75-77, the AAV particle of claim 78, or the LNP of claim 79 for genetically engineering a population of cells, whereby genomes of at least a plurality of the cells are altered.

93. Use of the Cas12a effector protein of any one of claims 1-71 and a gRNA molecule, the fusion protein according to claim 72 and a gRNA molecule, the polynucleotide of any one of claims 73-74 or the recombinant vector of any one of claims 75-77, the AAV particle according to claim 78, or the LNP of claim 79 for editing a population of double stranded DNA (dsDNA) molecules, whereby a plurality of the dsDNA molecules are edited.

94. Use of the Cas12a effector protein of any one of claims 1-71 and a gRNA molecule, the fusion protein according to claim 72 and a gRNA molecule, the polynucleotide of any one of claims 73-74 or the recombinant vector of any one of claims 75-77, the AAV particle of claim 78, or the LNP of claim 79 for treatment of a subject.

95. Use of the Cas12a effector protein of any one of claims 1-71 and a gRNA molecule, the fusion protein according to claim 72 and a gRNA molecule, the polynucleotide of any one of claims 73-74 or the recombinant vectors of any one of claims 75-77, the AAV particle of claim 78, or the LNP of claim 79 in the manufacture of a medicament for the treatment of a subject.
